(19) United States
(12) Patent Application Publication
(10) Pub. No.: US 2025/0250582 A1
(43) Pub. Date: Aug. 7, 2025

(54) Plant Cells and Plants Modified to Increase Herbicide Resistance and Stress Tolerance and Methods of Using the Same

(71) Applicant: The Board of Trustees of the University of Arkansas, Little Rock, AR (US)

(72) Inventors: Christopher E. Rouse, Fayetteville, AR (US); Nilda Roma-Burgos, Springdale, AR (US); Gulabben Rangani, Fayetteville, AR (US)

(73) Assignee: The Board of Trustees of the University of Arkansas, Little Rock, AR (US)

(21) Appl. No.: 19/009,491

(22) Filed: Jan. 3, 2025

Related U.S. Application Data
(63) Continuation of application No. 16/683,540, filed on Nov. 14, 2019, now abandoned.
(60) Provisional application No. 62/767,033, filed on Nov. 14, 2018.

(51) Int. Cl. C12N 15/82

(52) U.S. Cl. CPC C12N 15/8274; C12N 15/8273

(57) ABSTRACT

Plant cells, plants, seeds and portions of plants modified or genetically engineered to have increased expression or activity of at least one protein selected from the group consisting of trehalose phosphate synthetase (TPS), trehalose phosphate phosphatase (TPP), Protein ALP1-like (At3g55350 or ALPL1), Glycosyltransferase 75D1 (UGT75D1), Cytochrome P450 709B2 (CYP709B2), Cytochrome P450 709B1 (CYP709B1) and Cytochrome P450 72A15 (CYP72A15) are provided herein. Also provided are methods of increasing plant resistance to herbicides and/or abiotic stress by increasing expression of at least one of trehalose phosphate synthetase (TPS), trehalose phosphate phosphatase (TPP), Protein ALP1-like (At3g55350 or ALPL1), Glycosyltransferase 75D1 (UGT75D1), Cytochrome P450 709B2 (CYP709B2), Cytochrome P450 709B1 (CYP709B1) or Cytochrome P450 72A15 (CYP72A15).

### CROSS-REFERENCE TO RELATED APPLICATIONS

The present application is a continuation of U.S. patent application Ser. No. 16/683,540, filed Nov. 14, 2019, which application claims the benefit of priority to U.S. Provisional Patent Application No. 62/767,033, filed on Nov. 14, 2018, the contents of each of which are incorporated herein by reference in their entireties.

### SEQUENCE LISTING

This application includes a Sequence Listing in XML format titled “2025 Feb. 25 169946-00788_ST-26_Sequence_Listing_XML.xml”, which was created on Feb. 25, 2025 and is 75,959 bytes in size. The Sequence Listing is electronically submitted via Patent Center and is incorporated by reference herein in its entirety.

### INTRODUCTION

Herbicides are a cost-effective and widely used strategy for weed control. Often paired with cultivation or crop rotations, herbicide-based programs are often successfully used in crop production. For example, these management programs have been instituted in rice and soybean rotations to manage Echinochloa species and continue to be the standard weed management tool. In the 1950s, the first selective herbicide for Echinochloa control in rice, propanil, was released. To date, 10 rice herbicides from 5 mode-of-action categories have been commercialized, including an herbicide-resistant crop technology, Clearfield® rice, which allowed for the use of the highly efficacious herbicide, imazethapyr. These compounds were released over the course of 50+years, and were highly effective at their time of introduction. Due to their high efficacy and a lack of stewardship, these products soon became not just a capstone of weed management plan but the only strategy used.

The repeated and widespread use of these herbicides, however, has led to the evolved resistance to common rice herbicides such as propanil, quinclorac, imazethapyr, and cyhalofop. To mitigate resistance evolution, strategies such as diversification of herbicide modes of action, post-season and pre-season weed management activities, and crop rotation may be employed. Approaches such as these are effective at reducing the incidence of resistance but are often not integrated fully and consistently, allowing for continued selection pressure for resistance to herbicides. Thus, weed populations have become multiple-resistant, or resistant to herbicides of two or more modes of action. The increasing presence of these populations is a concern for producers and researchers as the underlying cause of resistance has yet to be understood and the threat of reduced efficacy to other herbicide products is of concern.

Mechanisms that enable herbicide resistance are broadly classified into two categories: target-site and non-target-site mechanisms. Target-site resistance is the modification of an herbicide site of action resulting in the reduced ability of the herbicide to interact with the target protein. This mechanism is specific to a single herbicide or group of herbicides from the same chemical family. Non-target-site mechanisms involve complex biological processes that result in either reduced herbicide reaching the target site, reduced herbicide activity, or enhanced physiological activity to allow for survival of the targeted species. This complex mechanism is not well understood and has resulted in broad resistance to herbicides from various modes of action and has led to reduced efficacy of herbicides without a history of use on weed populations. Accordingly, there remains a need in the art to understand these non-target-site mechanisms and potentially employ them in creating new crop varieties possessing unique herbicide resistance profiles and/or increased tolerance to abiotic stress.

### SUMMARY

In one aspect of the present invention, plant cells are provided. The plant cells may be modified to increase, as compared to a control plant cell, the expression or enzymatic activity of at least one, two, three, four, five, or six proteins selected from the group consisting of trehalose phosphate synthetase (TPS), trehalose phosphate phosphatase (TPP), Protein ALP1-like (At3g55350 or ALPL1), Glycosyltransferase 75D1 (UGT75D1), Cytochrome P450 709B2 (CYP709B2), and Cytochrome P450 72A15 (CYP72A15) (collectively, as used herein, the “Disclosed Proteins”).

In another aspect, plants are provided. The plants may include any one of the plant cells described herein.

In a further aspect, the present invention relates to methods of using the plants described herein. The methods may include planting any one of the plants and seeds described herein in an area. The plants described herein have increased resistance to abiotic stress, such as excessive heat or drought, and the plants have increased resistance to herbicides. Optionally, the methods may further include applying an herbicide to the plant in the area.

### BRIEF DESCRIPTION OF DRAWINGS

The patent or application file contains at least one drawing executed in color. Copies of this patent or patent application publication with color drawing(s) will be provided by the Office upon request and payment of the necessary fee.

FIG. 1 shows a depiction of the quinclorac-activated physiological pathway in E. colona following treatment including the attachment of quinclorac to the Transport Inhibitor Response 1 (TIR-1) DNA repressor complex and the activation of the 1-aminocyclopropane-carboxylase synthase enzyme leading to the build-up of ethylene and hydrogen cyanide in the plant.

FIG. 2 shows a treemap of the enriched gene ontology terms for ECO-R (E. colona from a a multiple-resistant population (ECO-R)) without herbicide treatment compared to ECO-S (E. colona from a susceptible population (ECO-S)) without herbicide treatment. Each box represents an ontological term and the size of the box depicts the p-value for the over-represented terms based on the analysis. The colors signify superclusters of loosely associated terms related via semantic analysis and identified by the description that is capitalized in bold. For example, a supercluster of terms identified as ‘trehalose metabolism in response to stress’ (purple) is enriched in ECO-R-N. Within this cluster are terms that include response to herbicide and nitrate, nitrate assimilation, positive regulation of transcription factor catabolic process, and trehalose metabolism in response to stress. Gene ontology (GO) provides classification of gene products into molecular function, biological processes and cellular components. For example, gene ontology annotation of a gene can be described by its molecular function (e.g. catalysis), biological process (e.g. oxidative phosphorylation) and cellular component (e.g. intermembrane space). A fundamental application of the GO is in the creation of gene product annotations, including evidence-based associations between GO definitions and experimental or sequence-based analysis. One type of visualization of GO annotation is a treemap. Treemaps are a space-filling visualization technique for hierarchical structures that show attributes by size and color-coding.

FIGS. 3A-3D show total fold change, both increasing and decreasing, for the gene families of xenobiotic detoxification enzymes categorized in the analysis for the differential gene expression analysis: (FIG. 3A) ECO-S-N vs ECO-R-N, (FIG. 3B) ECO-S-N vs ECO-S-T, (FIG. 3C) ECO-R-N vs ECO-R-T, (FIG. 3D) ECO-S-T vs ECO-R-T. treatment with quinclorac (ECO-R-T, ECO-S-T); treatment without quinclorac (ECO-R-N, ECO-S-N)

FIG. 4 shows a Venn diagram for the differential gene expression analysis with each oval (labeled A, B, C, and D) representing the number of repressed (A/B) or induced (C/D) genes within the comparisons of ECO-S-N vs ECO-R-N (A/C) and ECO-R-N vs ECO-R-T (B/D).

FIG. 5 shows a diagram depicting the proposed biological pathway for the conjugation of quinclorac via UGT75D1 (Glycosyltransferase 75D1) to the UDP-glucose (Uridine diphosphate glucose) molecule, which is a component of the trehalose biosynthetic process. The alternative substrates for the UGT75D1 enzyme—the endogenous molecule indole-3-acetic acid and exogenous xenobiotics kaempferol and 2,4,5-trichlorophenol, are presented as structural comparisons to quinclorac.

FIG. 6 shows proposed interconnected pathways describing the potential activities of ALPL1 (Protein ALP1-like) and UGT75D1, which work in concert to endow the quinclorac-resistant phenotype in ECO-R.

FIG. 7 shows a diagram depicting the quinclorac activated physiological pathway as explained by the literature and the response of ECO-S and ECO-R, 24-hours after treatment (HAT), as explained by the RNA-sequencing of the transcriptome.

FIG. 8 shows a diagram of the significantly enriched gene ontology terms in ECO-S following propanil treatment. The over-represented p-values are depicted by the intensity of the color of each oval and the arrows signify the relationship between each of the ontological terms. The location and relation of each oval and cluster signifies the relationships to the terms in semantic 2-dimensional space. For example, this biological characterization revealed several biological processes centered around inositol catabolism with enrichment in the sub-cluster-trehalose metabolism.

FIG. 9 shows a diagram of the significantly enriched gene ontology terms in ECO-R following propanil treatment. The p-value for the over-represented p-values is depicted by the intensity of the color of each oval and the arrows signify the relationship between each of the onotlogical terms. The location and relation of each oval and cluster signifies the relationships to the terms in semantic 2-dimensional space. This analysis revealed a supercluster identified as ‘trehalose metabolism in response to stress’ which was most over-represented of the multiple enriched terms.

FIG. 10 shows proposed two-phase detoxification pathway for propanil via phase 1 hydroxylation via CYP709B2 and/or CYP71A15 and phase II conjugation of glutathione and UDP-glucose to the substrates 3,4-dichloroanaline and propionic acid, respectively.

FIG. 11 shows a diagram of the physiological inhibitory pathways of cyhalofop, glufosinate, propanil, and quinclorac and the means through which they interconnect within the plant. Red stars within each of the labeled boxes indicate the site or enzyme the herbicides inhibit.

FIGS. 12A-12B show Venn diagrams depicting the number of shared and contrasting gene ontology terms that were enriched following herbicide application within either ECO-S (FIG. 12A) or ECO-R (FIG. 12B). Each oval represents a single herbicide response for the respective accession: cyhalofop (a), glufosinate (b), propanil (c), or quinclorac (d).

FIGS. 13A-13D show shared and unique genes for the repressed and induced genes in ECO-S and ECO-R following the differential gene expression analysis. FIGS. 13A and 13B are ECO-S repressed and induced genes, respectively; FIGS. 13C and 13D are ECO-R repressed and induced genes, respectively. Each oval represents a single herbicide: cyhalofop (a), glufosinate (b), propanil (c), or quinclorac (d).

FIG. 14 shows a graph showing the fold change in expression of the indicated cytochrome P450 genes in resistant (ECO45) plants relative to susceptible (ECOSS) plants.

FIG. 15 is a graph showing the fold change in expression of the indicated cytochrome P450 genes in resistant (ECO45) plants when treated with the indicated herbicide.

FIG. 16 shows a table depicting the fold change in the expression of six candidate genes (columns) across seven different Echinochloa genotypes with multiple resistance to propanil and quinclorac as compared to susceptible standard control plants (SS). The response to a 2× dose of propanil (P; 4480 g ai/ha) and quinclorac (Q; 1120 g ai/ha) are each in separate columns. Plants were treated at the three-leaf stage.

FIG. 17 shows bar graphs depicting the effect of water stress on the transpiration rate of multiple-resistant Echinochloa colona and rice based on a greenhouse study performed at Altheimer Laboratory complex (University of Arkansas, Fayetteville, AR, USA). E. colona accessions are as follows: 70 is a susceptible standard, 45 and 179 are multiple-resistant to propanil and quinclorac. MS (moderate stress): dry down to 50% of field capacity then rewater. SS (severe stress): dry down to 50% of field capacity and maintain at this level for two weeks. NS (no stress): well-watered.

### DETAILED DESCRIPTION

Here, in the non-limiting Examples, the present inventors conducted RNA-Seq experiments using herbicide-resistant and susceptible strains of Echinochloa colona, with and without treatment with cyhalofop, glufosinate, propanil, and quinclorac. They studied the function of differentially expressed genes based on gene ontology annotations, under various comparison scenarios, and identified putative primary candidate genes endowing resistance traits, potential biochemical pathway modifications, and connectivity across pathways that may lead to resistance to herbicides in new crop plants, low-level resistance to non-selector herbicides (resistance pre-conditioning), and high tolerance to abiotic stresses such as drought in new crop plants.

### Plant Cells

In one aspect of the present invention, plant cells are provided. The plant cells may be modified to increase, as compared to a control plant cell, the expression or enzyme activity of at least one, two, three, four, five, or six proteins selected from the group consisting of trehalose phosphate synthetase (TPS), trehalose phosphate phosphatase (TPP), Protein ALP1-like (At3g55350 or ALPL1), Glycosyltransferase 75D1 (UGT75D1), Cytochrome P450 709B2 (CYP709B2), Cytochrome P450 709B1 (CYP709B1) and Cytochrome P450 72A15 (CYP72A15) (collectively, as used herein, the “Disclosed Proteins”). Notably, any combination of the Disclosed Proteins may be used in combination. The inventors note that increased expression of the TPS and TPP in combination may increase expression of a pathway as discussed in the Examples section and is one embodiment of the invention. In other embodiments, the expression or activity of the TPS and TPP proteins may be increased in combination with at least one of the remaining disclosed proteins.

Trehalose phosphate synthetases (“TPS”) belong to the glycosyltransferase family of enzymes. In some embodiments, the TPS protein may include any one of SEQ ID NOS: 1-16 (Junglerice TPS) or a variant or homolog of any one of SEQ ID NOS: 1-16 including at least 60%, 70%, 80%, 85%, 90%, 95%, or 99% sequence identity to any one of SEQ ID NOS: 1-16.

Trehalose phosphate phosphatases (“TPP”) belong to the hydrolase family of enzymes. In some embodiments, the TPP protein may include any one of SEQ ID NOS: 17-21 (Junglerice TPP) or a variant or homolog of any one of SEQ ID NOS: 17-21 including at least 60%, 70%, 80%, 85%, 90%, 95%, or 99% sequence identity to SEQ ID NOS: 17-21.

Protein ALP1-like (At3g55350 or “ALPL1”) is predicted to belong to the hydrolase family of enzymes. In some embodiments, ALPL1 protein may include any one of SEQ ID NOS: 22-27 (Junglerice ALPL1) or a variant or homolog of any one of SEQ ID NOS: 22-27 including at least 60%, 70%, 80%, 85%, 90%, 95%, or 99% sequence identity to any one of SEQ ID NOS: 22-27.

Glycosyltransferase 75D1 (UGT75D1) is predicted to belong to the glycosyyltransferase family of enzymes. In some embodiments, the UGT75D1 protein may include any one of SEQ ID NOS: 28-30 (Junglerice UGT75D1), or a variant or homolog of any one of SEQ ID NOS: 28-30 including at least 60%, 70%, 80%, 85%, 90%, 95%, or 99% sequence identity to any one of SEQ ID NOS: 28-30.

Cytochrome P450 709B1 or 709B2 (CYP709B1 or CYP709B2) is member of the cytochrome P450 family of proteins. In some embodiments, the CYP709B2 may include any one of SEQ ID NOS: 31-33 (Junglerice CYP709B2), or a variant or homolog of any one of SEQ ID NOS: 31-33 including at least 60%, 70%, 80%, 85%, 90%, 95%, or 99% sequence identity to any one of SEQ ID NOS: 31-33. In some embodiments, the CYP709B1 may include SEQ ID NO: 34 (Junglerice CYP709B2), or a variant or homolog of SEQ ID NO: 34 including at least 60%, 70%, 80%, 85%, 90%, 95%, or 99% sequence identity to SEQ ID NO: 34.

Cytochrome P450 72A15 (CYP72A15) is member of the cytochrome P450 family of proteins. In some embodiments, the CYP72A15 protein may include any one of SEQ ID NOS: 35-39 (JungleRice CYP72A15), or a variant or homolog of any one of SEQ ID NOS: 35-39 including at least 60%, 70%, 80%, 85%, 90%, 95%, or 99% sequence identity to any one of SEQ ID NOS: 35-39.

In some embodiments, the plant cells may be modified to increase the expression of an ALPL1 protein. The present inventors have found, in part, that the expression of ALPL1 proteins, as well as UGT75D1 proteins, are significantly increased in plant cells following treatment with the various herbicides including quinclorac. Based in part on this data, the inventors predict that plant cells having increased expression of ALPL1 proteins or UGT75D1 proteins will be more resistant to herbicides such as quinclorac. ALPL1 is structurally similar to the Antagonist of Like Heterochromatin Protein 1 (ALP1) containing a unique harbinger transposase-derived nuclease domain (Liang et al. 2015). The harbinger transposase derived domain is a critical component of the ALPL1 proteins ability to target specific regions of the methylated DNA (Duan et al. 2017). Polycomb group (PcG) proteins associate with methylated sites along the heterochromatic regions of DNA, reducing the transcription of gene products, essentially serving as suppressor complexes of genes. ALPI is known to be associated with flowering proteins and the depression of the PcG complex results in the expression of flowering-related genes. Likewise, ALPI also has been observed to associate in critical growth regions of the genome, including cis-acting factors, which modulate physiological activities and result in pleiotropic effects (Ricci and Zhang 2016). Given the magnitude of the coordinated induction of ALPL1 and UGT75D1 across herbicide treatments, and the unique co-expression following quinclorac treatment, the present inventors predict, without being bound by theory, that ALPL1 proteins may be modulating UGT75D1 proteins to mount an effective herbicide response. In some embodiments, the plant cells may be modified to increase the expression of a UGT75D1 protein.

In some embodiments, the plant cells may be modified to increase the expression of a CYP709B2 protein. The present inventors have found, in part, that the expression of CYP709B2 proteins, as well as CYP72A15 proteins, are significantly increased in plant cells following treatment with the various herbicides including propanil. Based in part on this data, the inventors predict that plant cells having increased expression of CYP709B2 proteins or CYP72A15 proteins will be more resistant to herbicides such as propanil. The present inventors have found that herbicide resistance such as propanil resistance is endowed by the induction of a cytochrome P450 mechanism (i.e., through CYP709B2 and CYP72A15 proteins) that has not been previously investigated in crop species that are capable of hydroxylating propanil into a carboxylate and aniline. Based on this discovery, the present inventors predict that members of this pathway will provide proteins (i.e., CYP709B2 and CYP72A15 proteins) which can be expressed in crop species to enhance herbicide resistance as well as interact with the carbohydrate and sugar production within a plant species.

In some embodiments, the plant cells may be modified to increase the expression of a TPS protein and a TPP protein. The present inventors have found that the induction of TPS and TPP proteins, responsible for the production of trehalose, are necessary to not only supply trehalose but also several intermediary molecules required for abiotic stress tolerance. Based in part on this insight, the present inventors predict that trehalose induction prior to herbicide application will mitigate the effects of herbicide damage.

In some embodiments, the plant cells may be modified to increase the expression of a TPS protein, a TPP protein, a ALPL1 protein, a UGT75D1 protein, a CYP709B1 protein, a CYP709B2 protein, and a CYP72A15 protein.

As used herein, the terms “protein” or “polypeptide” or “peptide” may be used interchangeably to refer to a polymer of amino acids. A “protein” as contemplated herein typically comprises a polymer of naturally occurring amino acids (e.g., alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, and valine).

SEQ ID NOS: 1-39 are Disclosed Proteins identified in Echinochloa colona that may be used as reference sequences. SEQ ID NOS: 1-16 are protein sequences of TPS proteins. SEQ ID NOS: 17-21 are protein sequences of TPP proteins. SEQ ID NOS: 22-27 are protein sequences of ALPL1 proteins. SEQ ID NOS: 28-30 are protein sequences of UGT75D1 proteins. SEQ ID NOS: 31-33 are protein sequences of CYP709B2 proteins. SEQ ID NO: 34 is the protein sequence of CYP709B1. SEQ ID NOS: 35-39 are protein sequences of CYP72A15 proteins.

The Disclosed Proteins presented herein may include “variants” of SEQ ID NOS: 1-39 that are found in other varieties of Echinochloa colona or in other varieties of Junglerice in general. As used herein, a “variant” refers to a protein having an amino acid sequence that differs from a Disclosed Protein reference. A variant may have one or more insertions, deletions, or substitutions of an amino acid residue relative to a reference molecule. For example, a Disclosed Protein variant may have one or more insertion, deletion, or substitution of at least one amino acid residue relative to the reference Disclosed Proteins (SEQ ID NOS: 1-39) disclosed herein.

The Disclosed Proteins presented herein may include “homologs” of SEQ ID NOS: 1-39 that are found in other plant species besides Junglerice plants. A “homolog” may be a protein related to a second protein by descent from a common ancestral protein.

Regarding the Disclosed Proteins presented herein, the phrases “% sequence identity,” “percent identity,” or “% identity” refer to the percentage of residue matches between at least two amino acid sequences aligned using a standardized algorithm. Methods of amino acid sequence alignment are well-known in the art. A suite of commonly used and freely available sequence comparison algorithms is provided by the National Center for Biotechnology Information (NCBI) Basic Local Alignment Search Tool (BLAST), which is available from several sources, including the NCBI, Bethesda, Md., at its website. The BLAST software suite includes various sequence analysis programs including “blastp,” that may be used to align a known amino acid sequence with other amino acids sequences from a variety of databases.

Disclosed Protein sequence identity may be measured over the length of an entire defined polypeptide sequence, for example, as defined by a particular SEQ ID number, or may be measured over a shorter length, for example, over the length of a fragment taken from a larger, defined polypeptide sequence, for instance, a fragment of at least 15, at least 20, at least 30, at least 40, at least 50, at least 70 or at least 150 contiguous residues. Such lengths are exemplary only, and it is understood that any fragment length supported by the sequences shown herein, in the tables, figures or Sequence Listing, may be used to describe a length over which percentage identity may be measured.

A “deletion” in a Disclosed Protein refers to a change in the amino acid sequence resulting in the absence of one or more amino acid residues. A deletion may remove at least 1, 2, 3, 4, 5, 10, 20, 50, 100, 200, or more amino acids residues. A deletion may include an internal deletion and/or a terminal deletion (e.g., an N-terminal truncation, a C-terminal truncation or both of a reference polypeptide).

“Insertions” and “additions” in a Disclosed Protein refers to changes in an amino acid sequence resulting in the addition of one or more amino acid residues. An insertion or addition may refer to 1, 2, 3, 4, 5, 10, 20, 30, 40, 50, 60, 70, 80, 90, 100, 150, 200, or more amino acid residues. A variant or homolog of a Disclosed Protein may have N-terminal insertions, C-terminal insertions, internal insertions, or any combination of N-terminal insertions, C-terminal insertions, and internal insertions.

A “substitution” in a Disclosed Protein refers to a change of at least one amino acid in a sequence to another amino acid. A substitution may be a single amino acid substitution or more than one amino acid. A substitution may be a conservative change preserving the size and hydrophobicity of the original amino acid or may change either the length of the amino acid side chain or the charge or reactive group associated with the amino acid side chain.

As used herein, a “plant cell” may include any type of plant cell from any plant species. Suitable plants cells may include, without limitation, a corn plant cell, a cotton plant cell, a soybean plant cell, a rice plant cell, a wheat plant cell, a canola plant cell, a sugarbeet plant cell, a sunflower plant cell, a sorghum (grain or sweet sorghum) plant cell, a tomato plant cell, or a cucurbit plant cell.

The increased enzyme activity or expression of the Disclosed Proteins is relative to a control plant cell. A “control plant cell” is a plant cell that has not been modified as described herein. Exemplary control plant cells may include those from a natural plant species for a particular plant cell being modified.

As used herein, “enzyme activity” refers to the ability of a Disclosed Protein to catalyze a particular chemical conversion. In some embodiments, the enzyme activity of a Disclosed Protein may be increased by at least 30%, 40%, 50%, 55%, 60%, 65%, 70%, 75%, 80%, 85%, 90%, 95%, or more as compared to a control plant cell.

As used herein, the term “expression” may refer either to the levels of an RNA encoding a Disclosed Protein in a cell or the levels of the Disclosed Protein in a cell. In some embodiments, the expression of the Disclosed Protein is increased by at least 30%, 40%, 50%, 55%, 60%, 65%, 70%, 75%, 80%, 85%, 90%, 95%, or more as compared to a control plant cell.

The plant cells may be modified to increase, as compared to a control plant cell, the enzyme activity or expression of each of the Disclosed Proteins described herein. As used herein, the terms “modified” or “modifying” refer to using any laboratory methods available to those of skill in the art including, without limitation, genetic engineering techniques (i.e. CRISPR/Cas techniques or Recombinant DNA/transgenic technologies), traditional breeding/selection techniques, or forward genetic techniques to affect the enzyme activity or expression of a Disclosed Protein in a plant cell. It will be readily apparent to one of ordinary skill in the art that there are multiple potential ways to increase the enzyme activity or expression of a Disclosed Protein. These include, but are not limited to, modifying the gene encoding any one of these proteins by, for example, introducing targeted mutations; modifying a mRNA (or levels thereof) encoding any one of these proteins using, for example, transgenic techniques; or by enhancing the enzyme activity of the Disclosed Proteins at the protein level.

As used herein, the terms “polynucleotide,” “polynucleotide sequence,” “nucleic acid” and “nucleic acid sequence” refer to a nucleotide, oligonucleotide, polynucleotide (which terms may be used interchangeably), or any fragment thereof. These phrases also refer to DNA or RNA of natural or synthetic origin (which may be single-stranded or double-stranded and may represent the sense or the antisense strand). The polynucleotides may be cDNA or genomic DNA.

In some embodiments, the plant cell may include a heterologous promoter operably connected to a polynucleotide encoding a Disclosed Protein. As used herein, the terms “heterologous promoter,” “promoter,” “promoter region,” or “promoter sequence” refer generally to transcriptional regulatory regions of a gene, which may be found at the 5′ or 3′ side of the polynucleotides encoding a Disclosed Protein described herein, or within the coding region of such polynucleotides, or within introns of such polynucleotides. Typically, a promoter is a DNA regulatory region capable of binding RNA polymerase in a cell and initiating transcription of a downstream (3′ direction) coding sequence. The typical 5′ promoter sequence is bounded at its 3′ terminus by the transcription initiation site and extends upstream (5′ direction) to include the minimum number of bases or elements necessary to initiate transcription at levels detectable above background. Within the promoter sequence is a transcription initiation site (conveniently defined by mapping with nuclease S1), as well as protein binding domains (consensus sequences) responsible for the binding of RNA polymerase.

In some embodiments, the polynucleotides encoding a Disclosed Protein are operably connected to the heterologous promoter. As used herein, a polynucleotide is “operably connected” or “operably linked” when it is placed into a functional relationship with a second polynucleotide sequence. For instance, a promoter is operably linked to a polynucleotide encoding a Disclosed Protein if the promoter is connected to the polynucleotide such that it may effect transcription of the polynucleotide. In various embodiments, the polynucleotides may be operably linked to at least 1, at least 2, at least 3, at least 4, at least 5, or at least 10 promoters.

Heterologous promoters useful in the practice of the present invention include, but are not limited to, constitutive, inducible, temporally-regulated, developmentally regulated, chemically regulated, tissue-preferred and tissue-specific promoters. The heterologous promoter may be a plant promoter. Suitable promoters for expression in plants include, without limitation, the 35S promoter of the cauliflower mosaic virus, ubiquitine, tCUP cryptic constitutive promoter, the Rsyn7 promoter, pathogen-inducible promoters, the maize In2-2 promoter, the tobacco PR-1a promoter, glucocorticoid-inducible promoters, Ubi promoters, Act promoters, estrogen-inducible promoters and tetracycline-inducible and tetracycline-repressible promoters. Those of skill in the art are familiar with a wide variety of additional promoters for use in various cell types.

The plant cell may also be modified to introduce a hypermorphic mutation in a polynucleotide (i.e., gene) encoding a Disclosed Protein. A “hypermorphic mutation” is an alteration in a gene that results in a gene having increased function. The increased activity may be from an increased level of expression of gene products (i.e., protein or RNA) from the gene or may result from the expression of a gene product (i.e. protein or RNA) that have increased activity.

It will be readily apparent to those of skill in the art that a variety of hypermorphic mutations may be introduced (using, for example, CRISPR/Cas or other genome engineering techniques) into a polynucleotide encoding each of the Disclosed Proteins presented herein to arrive at embodiments of the present invention. For example, a person of ordinary skill may introduce alterations (i.e., substitutions or deletions) into the promoter of a polynucleotide encoding a Disclosed Protein presented herein that result in increased expression of the Disclosed Protein.

Still further modifications contemplated herein include mutations that impact one or more of the domains of the Disclosed Proteins. It will be understood by those of skill in the art that alterations (i.e., mutations and/or deletions) could be made in any one or more of these domains that would be expected to increase the enzyme activity of the Disclosed Protein.

### Plants

In another aspect of the present invention, plants are provided. The plants may include any one of the plant cells described herein. The plants may include plants in which every cell of the plant is a plant cell modified as described herein. Alternatively, the plants may include plants in which only certain tissues within the plant include the plant cells described herein. For example, with respect to transgenic techniques, it is contemplated that the plants may only have plant cells including a polynucleotide encoding a Disclosed Protein in certain tissues of the plant using, for example, tissue-specific promoters.

As used herein, a “plant” includes any portion of the plant including, without limitation, a whole plant or a portion of a plant such as a part of a root, leaf, stem, seed, pod, flower, tissue plant germplasm, asexual propagate, or any progeny thereof. For example, a corn plant refers to the whole corn plant or portions thereof including, without limitation, the leaves, flowers, fruits, stems, roots, or otherwise. Suitable plants may include, without limitation, a corn plant, a cotton plant, a soybean plant, a rice plant, a sorghum (sweet or grain) plant, a canola plant, a wheat plant, a sugarbeet plant, a tomato plant, a curcurbit plant or a sunflower plant.

The plant may exhibit improved properties over a control plant. For example, the plant may have improved resistance to an herbicide as compared to a control plant. Herbicide resistance may be measured using assays known in the art. The plants may exhibit improved resistance to abiotic stress such as drought or heat tolerance. The plants described herein may have improved drought tolerance and/or improved heat tolerance as compared to a control plant. Drought tolerance may be measured using methods known in art such as, for example, subjecting the plants to water-stress over a period of a certain number of days.

As used herein, an “herbicide” refers to a chemical compound that inhibits or kills a living plant cell. Suitable herbicides may include, without limitation, a photosynthesis inhibitor or a synthetic auxin. In some embodiments, the herbicide may be quinclorac, propanil, protoporphyrinogen IX oxidase (PPO) inhibitors, acetolactate synthase (ALS) inhibitors, photosystem I inhibitors, acetyl coenzyme A carboxylase (ACCase) inhibitors, fluorpyrauxifen, or carotenoid biosynthesis inhibitors, HPPD or DOXp synthase inhibitors.

As used herein, a “control plant” is a plant that has not been modified as described herein. Exemplary control plants may include those from a natural plant species for a particular plant being modified.

### Methods of Using

In another aspect, the present invention relates to methods of using the plants described herein. The methods may include planting any one of the plants described herein in an area. The area may be at risk of drought or other abiotic stress. Optionally, the methods may further include applying an herbicide to the plant in the area. The plants described herein have increased resistance to abiotic stress such as heat or drought and have increased resistance to herbicides and may be useful for planting in areas where overspray or drift from herbicide treated fields is possible.

As used herein, “applying” may be carried out through any of the variety of procedures used to apply herbicides to plants that will be apparent to the skilled artisan. Suitable application methods may include, without limitation spraying or dusting. Other suitable application procedures can be envisioned by those skilled in the art.

The present disclosure is not limited to the specific details of construction, arrangement of components, or method steps set forth herein. The compositions and methods disclosed herein are capable of being made, practiced, used, carried out and/or formed in various ways that will be apparent to one of skill in the art in light of the disclosure that follows. The phraseology and terminology used herein is for the purpose of description only and should not be regarded as limiting to the scope of the claims. Ordinal indicators, such as first, second, and third, as used in the description and the claims to refer to various structures or method steps, are not meant to be construed to indicate any specific structures or steps, or any particular order or configuration to such structures or steps. All methods described herein can be performed in any suitable order unless otherwise indicated herein or otherwise clearly contradicted by context. The use of any and all examples, or exemplary language (e.g., “such as”) provided herein, is intended merely to facilitate the disclosure and does not imply any limitation on the scope of the disclosure unless otherwise claimed. No language in the specification, and no structures shown in the drawings, should be construed as indicating that any non-claimed element is essential to the practice of the disclosed subject matter. The use herein of the terms “including,” “comprising,” or “having,” and variations thereof, is meant to encompass the elements listed thereafter and equivalents thereof, as well as additional elements. Embodiments recited as “including,” “comprising,” or “having” certain elements are also contemplated as “consisting essentially of” and “consisting of” those certain elements.

Recitation of ranges of values herein are merely intended to serve as a shorthand method of referring individually to each separate value falling within the range, unless otherwise indicated herein, and each separate value is incorporated into the specification as if it were individually recited herein. For example, if a concentration range is stated as 1% to 50%, it is intended that values such as 2% to 40%, 10% to 30%, or 1% to 3%, etc., are expressly enumerated in this specification. These are only examples of what is specifically intended, and all possible combinations of numerical values between and including the lowest value and the highest value enumerated are to be considered to be expressly stated in this disclosure. Use of the word “about” to describe a particular recited amount or range of amounts is meant to indicate that values very near to the recited amount are included in that amount, such as values that could or naturally would be accounted for due to manufacturing tolerances, instrument and human error in forming measurements, and the like. All percentages referring to amounts are by weight unless indicated otherwise.

No admission is made that any reference, including any non-patent or patent document cited in this specification, constitutes prior art. In particular, it will be understood that, unless otherwise stated, reference to any document herein does not constitute an admission that any of these documents forms part of the common general knowledge in the art in the United States or in any other country. Any discussion of the references states what their authors assert, and the applicant reserves the right to challenge the accuracy and pertinence of any of the documents cited herein. All references cited herein are fully incorporated by reference in their entirety, unless explicitly indicated otherwise. The present disclosure shall control in the event there are any disparities between any definitions and/or description found in the cited references.

Unless otherwise specified or indicated by context, the terms “a”, “an”, and “the” mean “one or more.” For example, “a protein” or “an RNA” should be interpreted to mean “one or more proteins” or “one or more RNAs,” respectively.

The following examples are meant only to be illustrative and are not meant as limitations on the scope of the invention or of the appended claims.

### EXAMPLES

### Example 1—High Resistance to Quinclorac in Multiple-Resistant Echinochloa colona Involves Adaptive Co-Evolution of Abiotic Stress- and Xenobiotic Detoxification Genes

Adaptation is a critical component of weed biology, allowing weedy species to respond to adversity and evolve to persist within agricultural landscapes. A unique multiple herbicide-resistant population of E. colona (ECO-R) was collected from a rice field in Arkansas, USA, and previously profiled for its level and mechanisms of resistance. Results from these experiments implicated an unknown xenobiotic detoxification enzyme as the cause of resistance to quinclorac, but further research into the specific gene was required. The following research presents the first de novo transcriptome from RNA-sequencing data and examination into the biological networks and gene expression patterns in a multiple-resistant and susceptible (ECO-S) E. colona. The de novo transcriptome identified 60,530 assembled genes from 109,539 transcripts. Constitutive gene expression, without herbicide treatment, was investigated between ECO-S and ECO-R implicating the induction of several plant growth and maintenance processes such as carbon metabolism and photosynthesis, as well as the trehalose biosynthetic processes, which were enhanced in ECO-R. Following herbicide treatment of ECO-S, 3,926 genes were induced, which included several xenobiotic detoxification genes and the induction of the established quinclorac mediated ethylene pathway. ECO-R response to quinclorac was much different, with only 74 genes being induced following treatment. One gene of interest, a glycosyltransferase gene-UGT75D1, was upregulated near 9-fold following quinclorac treatment. The high levels of trehalose induction prior to herbicide treatment and lack of change following treatment, indicates that a ready source of UDP-glucose could serve as the conjugate required for modification via UGT75D1. This mechanism may be due to the presence of ALPL1, an antagonist of an epigenetic repressor protein, induced by stress. This research provides the first characterization of the potential association between an abiotic stress mediating process-trehalose biosynthesis, and a xenobiotic detoxification gene-UGT75D1. The RNA-sequencing provides the first de novo transcriptome and subsequent global expression characterization of multiple-resistant E. colona.

Echinochloa spp. include highly diverse weedy members that are distributed globally, posing a threat to upland and lowland agricultural systems [1,2]. The genus is composed of several species well adapted to both dryland and flooded agriculture. Some species within the genus are cultivated as millet crops in underdeveloped regions, providing a needed nutrition source; but the majority are weedy and invasive [3]. While there is significant diversity within the genus, several species including the dominate E. colona (junglerice) and E. crus-galli (barnyardgrass) are phenotypically similar [4]. A history of co-domestication and continued selection in rice culture systems have resulted in crop mimics within these species [5,6]. In the US, 13 Echinochloa species have been recognized in 48 of the contiguous United States [7]. Of these, the most impactful in agricultural areas, specifically in rice and rice-based rotation crop systems, include E. colona, E. crus-galli, E. phyllopogon (late watergrass), and E. oryzoides (early watergrass). These species impact every major agricultural commodity including alfalfa, cotton, nut, perennial fruit, rice, soybeans, and several vegetable crops [8]. A single E. crus-galli plant has the ability to reduce yield by up to 65 kg ha-1; it is second only to weedy rice in terms of impact to production [9,10]

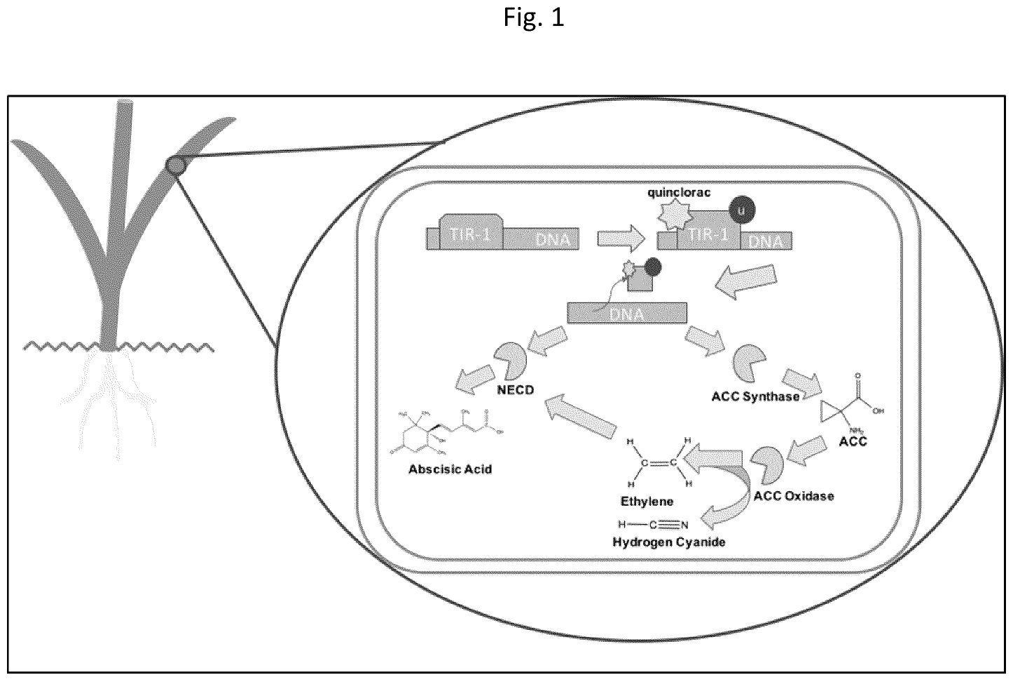

Rice production is considered minor in the US, however, it currently ranks third in export value contributing 10% of global exports [11]. In order to maximize production, weeds must be controlled as they are the most limiting biotic factor in rice production [1]. Propanil, a photosystem II inhibitor, was the first highly effective and selective Echinochloa herbicide in rice; this was followed by quinclorac, an auxin mimic, and several herbicide chemistries that disrupt fatty acid and amino acid synthesis [12]. Quinclorac has a unique mode of action in grass species that makes it highly effective on Echinochloa (FIG. 1). In dicots, quinclorac (like other auxin mimics, i.e., dicamba or 2,4-D) disrupts auxin regulation, causing elevated ethylene and abscisic acid (ABA) production, which results in uncontrolled cell elongation and growth, ultimately leading to plant death [13]. In monocots, quinclorac induces production of cyanide to toxic levels that results from excessive induction of ethylene in response to quinclorac [14]. Rice and other grass crops have a modification in the aminocyclopropane-1-carboxylic acid synthase (ACC synthase) enzyme that allows for selective induction of ACC synthase, providing insensitivity to the herbicide [15]. While the mechanisms have been described biochemically, transcriptome analysis may reveal details in the signal cascade that would improve or clarify our current understanding of how grass species respond, and adapt, to herbicidal auxin mimics.

Herbicide resistance in weedy species is an adaptive evolutionary trait selected for by repeated herbicide application. This is in contrast to herbicide tolerance in crop and weed species which results from underlying mechanisms that reduce herbicide response at the species level which develop independently and in the absence of herbicide selectors [16]. Two terminologies are used to describe herbicide resistance: target-site resistance (TSR) and non-target-site resistance (NTSR). TSR pertains to a modification in amino acid sequence of an enzyme the herbicide inhibits, resulting in reduced binding efficiency of the herbicide. NTSR encompasses diverse mechanisms including a number of physiological, biochemical, and structural responses that work via cascading processes leading to detoxification, redistribution, or sequestration of an herbicide, reducing the concentration of the herbicide at the site of action [17,18]. These mechanisms are the least understood and most problematic as they may result in broad-spectrum resistance to other herbicides and enhanced abiotic stress tolerance. Echinochloa species have evolved herbicide resistance using both mechanisms: TSR to multiple amino acid synthesis inhibitors [19], glyphosate and photosystem II inhibitors [21]; and NTSR to amino acid synthesis inhibitors, clomazone, propanil [22], and quinclorac [23].

Historically, research into the mechanisms of herbicide resistance has been limited to monogenic or single trait response characterization in weedy species. This is due to both a limitation in resources to investigate global genetic response patterns and a lack of understanding of the potential role that these responses may have on herbicide resistance. Evolution occurs through adaptive responses that modify existing biological pathways and the underlying processes that contribute to these pathways, allowing for survival. These modifications not only change the pathway, which is being acted upon, but also the interconnection of biological networks. Herbicide resistance traits do not evolve independent of other genetic and physiological factors. Research using advanced genomics techniques in weed science is currently limited; however, the demand for understanding herbicide resistance at a higher level will increase the utility of this type of research.

In this work, we present the first assembled transcriptome of E. colona from a susceptible (ECO-S) and a multiple-resistant (ECO-R) population under herbicide stress. We provide an in-depth characterization of the E. colona gene expression profiles and use this information to describe and compare the response of these biotypes to quinclorac. We identified and mapped the constitutive biochemical pathways that are involved in herbicide resistance and plant response to abiotic stress. This resistance mechanism is dependent on the constitutive induction of trehalose biosynthesis in the absence of the herbicide and the induction, following herbicide treatment, of a specific glycosyltransferase gene to conjugate the quinclorac molecule with UDP-glucose. The biochemical response of the resistant phenotype is vastly different from that of the susceptible one and demonstrates the divergence in evolution that occurs under immense herbicide selection pressure.

### Results

De Novo Transcriptome Assembly and Functional Characterization of E. colona

The de novo transcriptome assembled for E. colona represents two-week-old leaf tissue, 24 hours after treatment with (ECO-R-T) and without quinclorac (ECO-R-N). The transcriptome was assembled from 545,000,000 raw read pairs, which generated over 109,000 transcripts. Analysis of conserved plant ortholog sequences (BUSCO) revealed that approximately 75% of the transcriptome was resolved. Functional annotation revealed 60,530 genes retained, which were used to characterize the transcriptome. Homology to other organisms was as expected given the parameter of the annotation. However, sequence homology to Oryza sativa var. japonica (17.7%) is of value given the early co-domestication of these species, and their co-evolution throughout the history of rice production [5,6].

### Constitutive Difference in Gene Expression and Gene Networks Between ECO-S—N and ECO—R-N

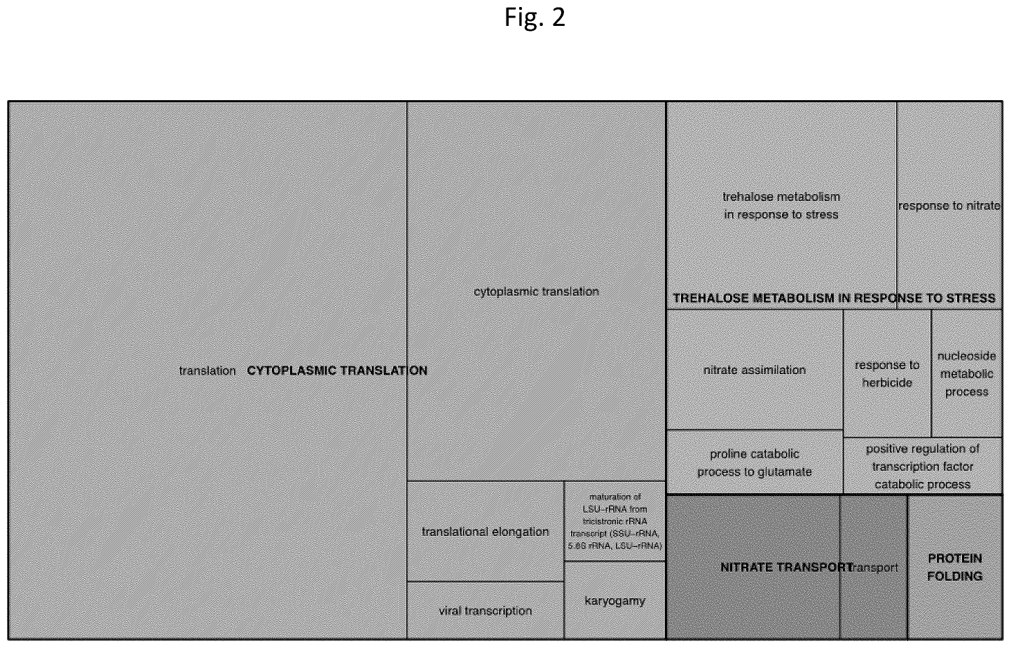

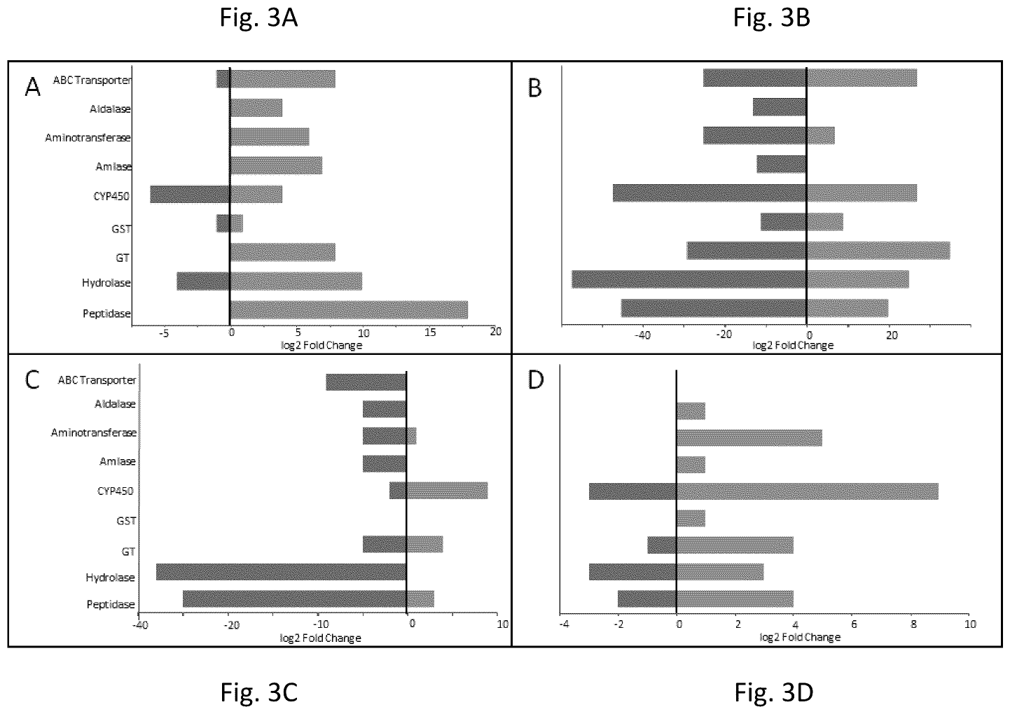

Gene network enrichment. Overall, transcription-, protein translation-, and protein synthesis-related terms were enriched in ECO-R-N and ECO-S-N (FIG. 2). However, the gene ontology analysis yielded several biochemical pathway features that are enriched for ECO-R-N relative to ECO-S-N. A supercluster of terms identified as ‘trehalose metabolism in response to stress’ was enriched in ECO-R-N. Within this cluster were terms that include response to herbicide and nitrate, nitrate assimilation, positive regulation of transcription factor catabolic process, and trehalose metabolism in response to stress. The nitrate responses were expected given the enriched nitrate transport and their connection to trehalose synthesis.
Plant growth and maintenance activity. Constitutive gene expression differed by 2,475 genes between ECO-R-N and ECO-S—N, with ECO-R-N having the greater gene expression (2,127); the majority of which were annotated (70%). Genes associated with growth functions such as carbon metabolism and photosynthesis were greatly enhanced in ECO-R-N. Photosynthesis-related genes such as ferredoxin-6 (4), ATP synthase subunits, (<4), NADH-cytochrome b5 reductase (5.4), and photosystem II core complex proteins psbY (4.8) were all elevated in ECO—R-N over ECO-S-N. Carbon assimilation genes were also induced: malate dehydrogenase (6.3), aspartate aminotransferase (5), phosphoenylpyruvate carboxylase (PEPC) kinase (4.7), pyruvate dehydrogenase subunits (>4.2), transketolase 1 (5.4) and the glycolysis component-triosephosphate isomerase (4.4). Both acetyl-CoA (3.4) and acetyl-CoA 2 (3.3) were induced. This indicates demand and utilization of products from carbon assimilation and photosynthesis in fatty acid metabolism. Nitrogen metabolism-related genes, specifically high affinity nitrate transporter-activating protein 2.1 (3.1 to 4.2), nitrate reductase (3.9 to 7.6), and glutamine synthetase (5.9) were induced. All these were indicative of higher level of biological activity in the resistant-than in the susceptible accession. Twelve DNA transcription factors, with ranging activities, were less abundant in ECO-R-N. Several MYB44 transcripts were induced (2.2 & 8.1). These have a role in abiotic stress response via ABA-inducible processes under drought stress [24]. The elevated activities of DNA ligase (4.2), DNA repair protein RAD16, and several DNA polymerase proteins, indicate higher-level activities of ECO-R-N.
Sugar metabolism and transport activity. Trehalose metabolism was a biological function supercluster that was significantly enriched containing multiple GO terms. Twenty-three transcripts, for eight genes in the trehalose pathway were enhanced in ECO-R-N compared to ECO-S-N. Five were a, a-trehalose-phosphate synthase UDP-forming enzymes (TPS) and three were probable trehalose-phosphate phosphatases (TPP). These genes all feature in abiotic stress response and stress tolerance [23]. Their enhanced constitutive expression in ECO-R-N is unique given these plants were not grown under stress. Sugar transport protein 14 (3.3 & 7) and bidirectional sugar transporter SWEET2a (3.3) were also induced, serving as transporters for this elevation in trehalose sugar quantities.
Ethylene biosynthetic pathway activity. Induction of the ethylene biosynthetic pathway is a major component of plant response to quinclorac. The activity of ACC-synthase was repressed in ECO-R-N (−7.4). Two forms of ACC-oxidase homolog 11 were observed, one was repressed (−3.4) and the other enhanced (2.3). Several ethylene-responsive transcription factors (ERF) were constitutively expressed, indicating heightened transcriptional activity to effect ethylene-mediated responses. Six ERFs were repressed, all involved in transcriptional repression, while 11 ERFs related to transcriptional activation were enhanced. The majority of these ERFs, both repressors and activators, bind to the GCC-box pathogenesis-related promoter element. This promoter element is linked to stress tolerance and signal transduction in response to disease, cold, salt, and/or water deprivation stress [25,26]. In ECO-R-N an elevated ethylene insensitive protein (EIN) 3 (2.7) gene was present; when in complex with ERF1 (2.5), both acts as component in ethylene signal transduction, bacterial defense, and hypoxia response, as well as sugar mediated signaling [27].
Xenobiotic detoxification genes present at the constitutive level. Genes within several xenobiotic detoxification gene families were differentially expressed in ECO-R-N relative to ECO-S-N (Table 1; FIG. 3A). Seven ABC transporters were identified, six of which were enhanced. Three cytochrome P450 enzymes were enhanced in ECO-R, including CYP90D2, CYP94C1, and CYP71A21. CYP90D2 is a component of brassinosteroid synthesis; CYP94C1 is involved in the oxidation of the phytohormone jasmonyl-L-isoleucine and wound response [29]; and CYP71A21 is involved in secondary metabolite biosynthesis with no described functions. The cytochrome P450 enzymes are associated with transmembrane movement of compounds and phytohormones. Their constitutive upregulation indicates possible involvement in intrinsic stress tolerance in ECO-R.

TABLE 1
Expression summary of detoxifying gene families and
subsequent genes involved in xenobiotic detoxification
differentially expressed in ECO-R compared to ECO-S

| | | Fold |
|---|---|---|
| Gene Family | Gene | Change |
| ABC | B family member 11 | −5 |
| Transporter | B family member 6 | 2 |
| | G family member 53 | 2 |
| | F family member 1 | 4 |
| | D family member 2 | 4 |
| | F family member 4 | 5 |
| | G family member 48 | 8 |
| Amino- | Alanine--glyoxylate aminotransferase | 3 |
| transferase | 2 homolog 3, mitochondrial | |
| | Aspartate aminotransferase, | 5 |
| | mitochondrial | |
| | Probable alanine aminotransferase, | 5 |
| | mitochondrial | |
| Amylase | Beta-amylase 3, chloroplastic | 2 |
| | Alpha-amylase isozyme 3D | 4 |
| Cytochrome | 72A14 | −9 |
| P450 | 72A15 | −8 |
| | 89A9 | −8 |
| | 76C1 | −4 |
| | 71A1 | −3 |
| | 90D2 | 4 |
| | 94C1 | 5 |
| | 71A21 | 6 |
| GST | Glutathione S-transferase 1 | −3 |
| | Glutathione S-transferase T3 | 11 |
| GT | UDP-glycosyltransferase 74D1 | 3 |
| | UDP-glycosyltransferase 83A1 | 3 |
| | UDP-glycosyltransferase 73C2 | 4 |
| | UDP-glycosyltransferase 75C1 | 4 |
| | UDP-glycosyltransferase 73C1 | 4 |
| | UDP-glycosyltransferase 90A1 | 6 |
| | UDP-glycosyltransferase 73E1 | 6 |
| Hydrolase | Ubiquitin carboxyl-terminal hydrolase 27 | −7 |
| | Uncharacterized abhydrolase domain- | −5 |
| | containing protein | |
| | Nudix hydrolase 21, chloroplastic | −3 |
| | Haloacid dehalogenase-like hydrolase | −2 |
| | domain-containing protein 3 | |
| | Probable xyloglucan endotransglucosylase/ | 2 |
| | hydrolase protein 30 | |
| | Putative aminoacrylate hydrolase RutD | 3 |
| | Xyloglucan endotransglucosylase/ | 3 |
| | hydrolase protein 22 | |
| | Hydrolase C26A3.11 | 4 |
| | IAA-amino acid hydrolase ILR1-like 4 | 4 |
| | Pyrimidine-specific ribonucleoside | 4 |
| | hydrolase RihA | |
| | Ubiquitin carboxyl-terminal hydrolase 6 | 4 |
| | 3-hydroxyisobutyryl-CoA hydrolase, | 9 |
| | mitochondrial | |
| Peptidase | Serine carboxypeptidase-like 27 | 3 |
| | Serine carboxypeptidase-like 18 | 3 |
| | Aspartyl aminopeptidase | 3 |
| | Leucine aminopeptidase 2, chloroplastic | 4 |
| | Carboxypeptidase 1 | 4 |
| | Mitochondrial-processing peptidase subunit alpha | 4 |
| | Serine carboxypeptidase-like 49 | 4 |
| | Probable cytosol aminopeptidase | 4 |
| | Prolyl endopeptidase | 4 |
| | Mitochondrial-processing peptidase subunit beta | 4 |
| | Puromycin-sensitive aminopeptidase | 5 |
| | Methionine aminopeptidase 2 | 5 |
| | Mitochondrial intermediate peptidase | 5 |
| | Thimet oligopeptidase | 5 |
| | Probable aminopeptidase NPEPL1 | 5 |
| | Dipeptidyl aminopeptidase BI | 5 |
| | Cytosolic non-specific dipeptidase | 6 |

Two glutathione-S-transferase (GST) enzymes were identified as well. One was GST1, which aids in glutathionylation of proteins, and the other was GST-T3, which conjugates glutathione to various hydrophobic electrophiles. GST-T3 has been implicated in detoxification of herbicides based on its sequence similarities to like proteins within the Uniprot database. Seven glucosyltransferase (GT) enzymes were induced in ECO-R. Three of these (UGT83A1, UGT73C2, and UGT90A1) are involved in the transfer of the glucosyl group from UDP-glucose to either the 3- or 7-hydroxy group on the quercetin molecule; a flavonol with auxin transport inhibitor and antioxidant activities. UGT73C1, has similar quercetin activity but also transfers glucose to cis- and trans-zeatin and can detoxify 2,4,6-trinitrotoluene (TNT) in plants by forming O- or C-glucosides [30]. UGT74D1, is unique in that it glycosylates indole-3-acetic acid (IAA), a natural auxin similar to quinclorac. Several aminotransferase, amylase, hydrolase, and peptidase enzymes were also expressed, indicating active modifications of biomolecules. These enzymes may also be involved in natural growth processes, but are not necessarily related to herbicide resistance.

Plant abiotic stress signaling activities. Several biological pathways, including some of the aforementioned gene families and genes of the auxin-, peptide-, and abscisic acid response pathways, are involved in plant signaling [31-33]. Enhanced auxin response factors such as ARFSAUR72, ARF13, IAA19, and IAA30 primarily serve as transcription factors that bind to promoter sequences, modulating gene expression following auxin signaling. In the quinclorac response pathway the production of ABA results in stomatal closure, limiting photosynthetic activity and disrupting electron flow in the photosystem complexes, causing irreparable cellular damage [32,34]. Disruptions in ABA signaling could be a source for limiting the negative effects caused by herbicide application. This would result in less stomatal closure, which may lead to a build-up of free energy which results in cell membrane disruption. Two ABA receptor proteins PYL8 (3.8) and PYL5 (2.1), had enhanced expression in ECO-R, indicating that the plant is producing the necessary components to receive ABA signals. However, four forms of ABA 8′-hydroxylase 1 were also enhanced (4.5 to 6.4-fold change); these are oxidative enzymes involved in catabolizing ABA. This means that although some ABA receptors are produced, there was insufficient ABA to transport. Several calcium receptors, components of the ABA signaling process [36], were repressed in ECO-R: CML45, CML46, and CRLK1. CRLK1 is unique in that it is also required for cold tolerance, which is enhanced by increasing calcium concentrations. The majority of the calcium-signaling-related genes are involved in transport or calcium perception. CPK5 (2.2), is a receptor that regulates reactive oxygen species (ROS) by directing kinase activity to the NADPH-oxidase [37]. Given the elevated levels of ABA-catabolizing enzymes and a reduction in several calcium receptors, it is possible that ECO-R is less sensitive to stress-induced cellular destruction through avoidance mechanisms [36]. One protein of note, with several transcripts constitutively repressed (−12 to −7) and enhanced (3.5 to 6.4) in ECO-R-N, is protein ALP1-like (ALPL1). Not much is known about ALPL1 other than it is analogous to the ALPI protein, which is a stress-responsive transcription factor that antagonizes Polycomb group (PcG) proteins [38,39]. PcG proteins rest on sections of target DNA repressing the transcription of the subsequent proteins. ALPL1 may possess significant epigenetic functions that may assist in the herbicide resistance response via activation of DNA segments allowing for transcription of needed genes and enzymes.

### Coordinated Gene Expression Following Quinclorac Treatment in ECO-S

Gene network enrichment. ECO-S-T had enriched GO terms for 25 biological functions, 2 cellular components, and 16 molecular functions (Table 2). The frequency is presented and provides information on the frequency of the GO term in the underlying GOA database, the lower the value the more unique and specific the term is for its function [40]. The ethylene-activated signaling pathway and the abscisic acid-activated signaling pathway were enriched, both of which would be a direct response to the herbicide. Anaerobic respiration, detection of hypoxia, response to hypoxia, response to oxidative stress, and the oxidation-reduction enriched terms indicate severe abiotic stress. Molecular function terms related to stress response were enriched including oxidoreductase, heme binding, peroxidase, and ABA 8′-hydroxylase activities. The enrichment of heme binding and oxidoreductase activity implies that cytochrome P450 enzymes, which are primary agents of phase I degradation of xenobiotic compounds, were induced following treatment. Nitrate assimilation GO terms similar to those observed in ECO-R-N were also enriched in response to quinclorac.

TABLE 2
Enhanced gene ontology terms and the
respective frequencies identified in ECO-S
following quinclorac application.

| GO Type | GO Term | Frequency¹ |
|---|---|---|
| Biological | protein phosphorylation | 4.14% |
| Process | detection of hypoxia | 0.00% |
| | response to hypoxia | 0.05% |
| | anaerobic respiration | 0.05% |
| | hydrogen peroxide catabolic process | 0.09% |
| | nitrate assimilation | 0.09% |
| | oxidation-reduction process | 15.06% |
| | cell surface receptor signaling pathway | 0.92% |
| | auxin catabolic process | 0.00% |
| | salicylic acid catabolic process | 0.00% |
| | peptidyl-cysteine oxidation | 0.00% |
| | response to bacterium | 0.15% |
| | defense response to bacterium | 0.10% |
| | ‘de novo’ CTP biosynthetic process | 0.07% |
| | pyrimidine nucleobase biosynthetic process | 0.24% |
| | abscisic acid-activated signaling pathway | 0.01% |
| | regulation of salicylic acid mediated | 0.01% |
| | signaling pathway | |
| | response to jasmonic acid | 0.01% |
| | defense response | 0.57% |
| | abscisic acid catabolic process | 0.00% |
| | protein autophosphorylation | 0.08% |
| | response to oxidative stress | 0.58% |
| | defense response to oomycetes | 0.00% |
| | response to oomycetes | 0.00% |
| | ethylene-activated signaling pathway | 0.01% |
| Molecular | transcription factor activity, sequence- | 4.22% |
| Function | specific DNA binding | |
| | protein kinase activity | 3.39% |
| | protein serine/threonine kinase activity | 1.00% |
| | symporter activity | 0.29% |
| | polysaccharide binding | 0.10% |
| | CTP synthase activity | 0.04% |
| | peroxidase activity | 0.38% |
| | heme binding | 1.36% |
| | sequence-specific DNA binding | 2.22% |
| | ATP binding | 14.13% |
| | (+)-abscisic acid 8′-hydroxylase activity | 0.00% |
| | alcohol dehydrogenase (NAD) activity | 0.04% |
| | oxidoreductase activity | 0.18% |
| | inositol oxygenase activity | 0.01% |
| | oligopeptide transmembrane transporter activity | 0.01% |
| | cysteine dioxygenase activity | 0.00% |

¹Frequency is the percentage of proteins in UniProt which were annotated with terms in the underlying GOA database, lower frequency indicates very specific terms while higher values indicate more general terms.

Quinclorac response pathway. Three transcripts within the ethylene response pathway were repressed following treatment: ACC synthase (−2.7), ACC oxidase (−2.8), and ACC oxidase homolog 3 (−2.2). Several transcripts were induced: two forms of ACC oxidase 1 (2.2 and 2.8) and four forms of ACC oxidase homolog 11 (1.6 to 6.4). The repression of ACC synthase, paired with the induction of multiple ACC oxidase transcripts, reflects the increase in ACC synthesis following quinclorac treatment. VP14 (9-cis-epoxycarotenoid dioxygenase [NECD]) is the first enzyme in ABA biosynthesis and is also a component of plant response to quinclorac [14]. Following treatment, VP14 was repressed (−6.4), indicating that by 24 hours sufficient ABA had been synthesized and feedback inhibition was occurring. Twelve ERF genes were repressed following treatment and 15 ERF genes were enhanced, similar to what was observed in ECO-R-N. The majority of the repressed genes were transcriptional repressors and the induced genes were transcriptional activators that interact with the GCC-pathogenesis promoter involved in stress signaling in plants. EIN2 (3.9) is a unique central factor in many signaling pathways including those related to plant development and defense as well as gene regulation and perception of environmental cues [41]. RAP2-2, another unique enzyme with enhanced expression (1.9), is a transcriptional activator for the promoter of phytoene synthase and desaturase enzymes in the carotenoid biosynthetic pathway [42]. This response has not been described previously; however, it is expected given the downstream effect of ABA synthesis resulting in stomatal closure. This has the potential to lead to an accumulation of light energy producing free radical or reactive oxygen species (ROS) following herbicide treatment.
Herbicide detoxification gene expression following quinclorac treatment. A total of 210 genes categorized as components of the detoxification process were identified in ECO-S-T (FIG. 3B). Fifty-two transcripts representing 33 ABC transporter genes were identified. The ABC transporters characterized in ECO-S-T perform various biological compound movement activities. ABCB5 (−6.5) has known auxin efflux transport activity and ABCC10 (−6.1), as well as several other repressed proteins, are glutathione S-conjugate pumps based on sequence homology. Seventy-four cytochrome P450 enzyme transcripts were differentially expressed; 47 were repressed. Sixteen annotated cytochrome P450 genes were induced (1.5 to 7.8). These have roles in secondary metabolite biosynthesis, brassinosteroid biosynthesis, and stress response. Eleven transcripts, representing six repressed genes and four induced genes, were GST enzymes. The induced genes-GSTT3, GSTU8, and GST4, involve the conjugation of glutathione to hydrophobic electrophiles. The gene family of note, GT, comprised the second most observed transcripts (64) following treatment. UGT83A1, UGT74D1, UGT75C1, UGT73E1 were upregulated in both ECO-S-T and ECO-R-N, indicating their involvement in plant maintenance but also possibly in general plant stress response. The majority of these GT enzymes are involved in glycosylation to C- and O-side groups. Two genes of note, UGT74F2 and UGT74E2, have known interactions with auxin compounds, like quinclorac. UGT74F2 glycosylates benzoic acid and benzoic acid derivatives, similar to the herbicide dicamba, another plant growth regulator used in weed management [43]. UGT74E2 interacts with endogenously produced indole-3-butyric acid (IBA) altering auxin homeostasis which results in stress-induced morphology changes [44]. The variety and high quantity of xenobiotic detoxification transcripts observed following quinclorac treatment again indicate coping mechanisms against elevated stress, none of which were effective for quinclorac detoxification.
Stress responsive genes and signaling response. To best characterize the whole plant response to quinclorac, we need to study stress-specific genes. These stress-responsive genes may produce a wide variety of proteins that could potentially stabilize cellular structure and function or facilitate stress signaling. In total, 247 transcripts that could be categorized as abiotic or biotic stress proteins were differentially expressed; 99 were repressed and 142 were induced following treatment. Fifty-eight disease resistance genes with hypersensitive activity in response to bacterial avirulence proteins were enhanced. The hypersensitive response, which results from a buildup of hydrogen peroxide, could potentially limit the movement of herbicide in the plant [45]. Eight heat shock proteins were induced. A total of 64 peroxidase transcripts were induced (3.1 to 8). Peroxidases are protection agents against cellular damage by free radicals. Abscisic stress-ripening protein 1 (ASR1) was also enhanced. This is associated with plant response to water deprivation, a process that leads to enhanced ABA production to mitigate water loss [46]. Five transcripts for ALPL1 were repressed by as much as −12 to −1.4-fold while three transcripts for were induced but only to as much as 3.5-fold.
Coordinated gene expression following quinclorac treatment in ECO-R
Gene network enrichment. None of the GO terms described in the ECO-S response to quinclorac were observed in ECO-R-T. In ECO-R-N, the majority of the enriched terms were involved with plant growth and maintenance processes, having no relationship to herbicide response. Fifty-three terms were significantly depleted in ECO-R-N relative to ECO-R-T. While most were irrelevant, several were related to stress responses including cold stress-, ABA-, and salicylic acid genes; plant-type hypersensitive response; and general plant defense response. No terms were enriched in ECO-R-T.
Plant growth and maintenance processes. The majority of genes coding for proteins in major metabolic pathways (photosynthesis, carbon metabolism, respiration, and fatty acid synthesis) were repressed in ECO-R-T; none were induced. Among the repressed transcripts were ATP synthase subunit (up to −12.3-fold), pyruvate dehydrogenase subunits (up to a −12-fold), cytochrome c oxidase proteins (−8.4-fold), and both acetyl-CoA and acetyl-CoA 2 proteins (up to −10.2-fold). In general, following quinclorac application, ECO-R appears to repress all non-essential processes.
Quinclorac-mediated response. Many genes in ECO-R were downregulated following treatment (5,311 transcripts), and only a minimal increase in gene expression (74 transcripts) was observed. This pattern of expression implies that the constitutive upregulation of certain genes is a major mechanism contributing to quinclorac resistance in this plant. ACC synthase was upregulated 6.3-fold in ECO-R-T. Given that there were no differences in the ACC oxidase transcripts without quinclorac, it appears that the plant is responding to quinclorac, but without the expected overload of ethylene. In ECO-R-T ALPL1 was present with a greater abundance in transcripts-7.4-fold upregulation, implicating it in the E. colona response to quinclorac. However, given the significant increase in expression its potential value in ECO-R-T must be considered.
Xenobiotic detoxification gene expression. Over 100 detoxification-related transcripts were differentially expressed following quinclorac treatment; 84 were repressed and 17 were induced (FIG. 3C). Only one of the ABC transporters, ABCD2 (−9.2) which were elevated in ECO-R-N was repressed following treatment. Four cytochrome P450 genes were upregulated following treatment: CYP709B1, four forms of CYP709B2, three forms of CYP72A15, and CYP89A2. CYP72A15 is the only gene, which was constitutively upregulated in ECO-R-N and upregulated further in ECO-R-T. This may indicate its necessity following herbicide application or that it is stabilizing an effected plant process. The three remaining CYP genes have stress response properties, potentially involved in phase I chemical degradation. UGT73D1 (5.3) with quercetin O-activity and UGT75D1 (7.3) with potential xenobiotic detoxification activity, based on homology, were upregulated in response to quinclorac. UGT75D1, with the greatest induction, is uniquely involved in glycosylation of indole-3-acetate, which is a growth hormone that is structurally analogous to quinclorac [47]. UGT73E1 was upregulated (2.6) following treatment in ECO-R-T. Transcripts for this gene were enhanced in ECO-R-N (6.3) and in two forms were present in ECO-S-T (−4.6 and 5.9). This gene may be involved in herbicide resistance given its elevated expression in ECO-R and repression in ECO-S following treatment. Given that multiple transcripts for various forms of the gene are present, the polyploidy of E. colona may have a role in its action and the genome from which this gene is expressed may play a role in resistance.

### Comparative Network Enrichment and Gene Expression Following Quinclorac Treatment Between ECO-S-T and ECO-R-T

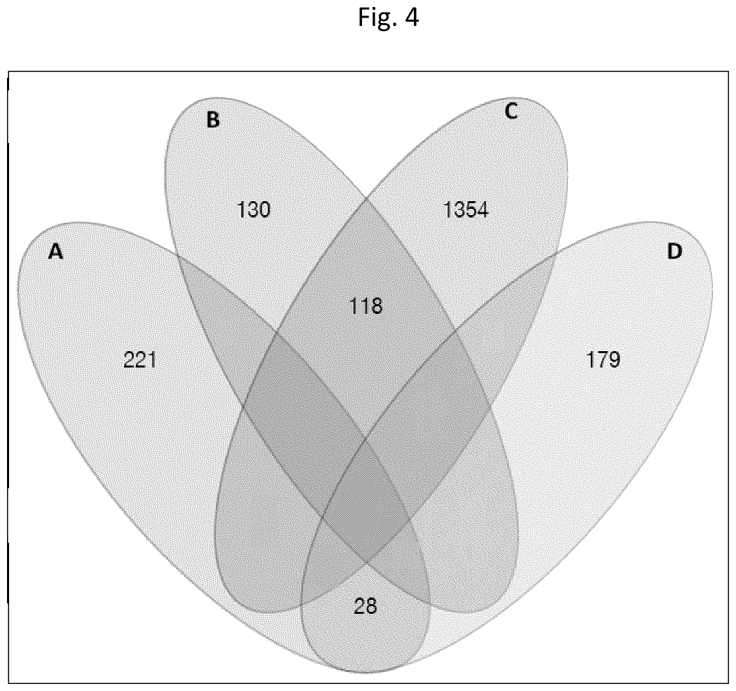

Gene network enrichment. Comparison between ECO-S-T and ECO-R-T revealed five ontological terms that were enriched in ECO-R-T related to carbohydrate biosynthesis: galactose-1-phosphate guanalyltransferase activity, GDP-L-galactose phosphorylase activity, GDP-D-glucose phosphorylase activity, and the reductive pentose-phosphate cycle. The term L-amino acid efflux transmembrane transporter was also enriched in ECO-R-T. No other enriched or depleted terms for ECO-N-T or ECO-R-T were present in the comparison.
Quinclorac response pathway. Comparison across both the treated samples provides an indication of the mechanisms that may be involved in herbicide resistance and/or general stress tolerance. In total, 595 transcripts were differentially expressed, 326 of which were repressed and 269 were upregulated. 118 transcripts that were enhanced constitutively were repressed following treatment; 28 were that were repressed were enhanced following treatment in ECO-R (FIG. 4). Of the 28 enhanced transcripts, one of note was the increased expression of ACC synthase (9), further suggesting that the quinclorac is reaching the target. There were six ERF transcripts induced for three genes: ERF4, EF8, and multiple forms of ERF11, all of which bind to the GCC-box pathogenesis promoter involved in stress response and signal transduction. ERF11 and ERF8 are transcriptional promoters while ERF4 is a repressor.

As previously noted, only one gene of significance within the ethylene pathway was induced at a higher level in ECO-R-T compared with ECO-R-N, ACC synthase (9), and again it was noted compared with ECO-S-T. The aforementioned VP14, involved in ABA synthesis and induced in the quinclorac-mediated pathway, was not observed in this comparison but four forms of ABA 8′-hydroxylase were present at lower levels. Given this pattern, it did not appear as though there was a significant induction of the NECD required for ABA synthesis nor were the ABA concentrations high enough to warrant the hydroxylase enzyme. PYL5, an ABA receptor protein was also significantly repressed, −4.4-fold lower, in ECO-R-T compared to ECO-S-T. Reductions in ABA synthesis and reduced perception may increase abiotic stress tolerance and reduce the negative effects of herbicide application. The collective pattern of gene expression indicates that ECO-R-T is perceiving quinclorac at its expected target; however, there appears to be a significant reduction in auxin perception and signaling, which was reflected in reduced plant response to quinclorac.

Stress signaling. Several stress-related proteins were comparatively expressed including RVE2, part of cold-responsive gene expression and a response to auxin, NHL3 a bacterial resistance gene induced in response to wounding, and FABIC a phosphorylating enzyme involved in stomatal closure. Several transcripts of note, which were comparatively repressed following treatment, include RVE1, ILL4, ARF13, and ERF113. RVE1 regulates free auxin levels in a time-of-day manner and is a negative regulator of freezing tolerance, a counter to RVE2. ILL4 is a hydrolyzing enzyme of amino acid conjugates involving IAA, which may be of note considering the imbalanced perception of auxin caused by quinclorac. This is also evident in the repression of ARF13 transcripts whereby the auxin mediated pathways are not responding at the same level in ECO-R-T as they are in ECO-S-T. ERF113 is transcriptional activator involved in plant development and tolerance to abiotic stress specifically waterlogging; the expression of this gene in ECO-S-T and comparative repression in ECO-R-T further indicates a reduction in auxin signaling, which would be caused by a reduction in overall auxin perception. While little is known about the function of ALPL1 it is evident based on the transcriptome profile, with transcripts ranging from 10.6 to 19.4-fold differences between ECO-S-T and ECO-R-T, that it possesses a major role in the ECO-R response to quinclorac. This high level of expression is the greatest among all transcripts in this different comparison condition.
Xenobiotic detoxification differences following quinclorac treatment. A greater number of xenobiotic detoxification genes were induced ECO-R-T than ECO-S-T, several of which had forms both repressed and induced (Table 3, FIG. 3D). Aldolase, aminotransferase, amylase, hydrolase, peptidase genes are involved in the transfer of their respective conjugates or peptides to other proteins, which may or may not be directly involved in herbicide metabolism. While these may have a role in xenobiotic metabolism given the literature, more research needs to be conducted to adequately describe their roles in herbicide resistance. CYP89A1, CYP72A15, and CRYP71A9 were all repressed in ECO-R-T compared to ECO-S-T, consequently they are not involved in herbicide resistance. All of the cytochrome P450 genes with increased expression have been previously discussed and play a role in stress response except for CYP71A4. CYP71A4 was expressed to a greater extent in ECO-R-T (5.6) and has been described has having a role in maturation and metabolite production in older tissues [48]. This is interesting because these are young tissues and most maturation and secondary metabolite synthesis can be directed by ethylene under abiotic stress conditions. CYP71A1 was induced following treatment and is involved in the oxidation of flavoproteins during the fruit ripening process, this is important as this would indicate an ethylene induced response also characteristic of quinclorac activity [49]. CYP709B2 induction is also of interest given its induction by ABA and salt stress, both abiotic signals for plant response [50]. GSTU20, involved in toxic substance response and far-red light influence on development was present in ECO-R-T with a significantly higher number of transcripts (6). The same GT enzymes enhanced in ECO-R-N and induced in ECO-R-T, compared with ECO-R-N, were present at higher levels in ECO-R-T compared with ECO-S-T. UGT75D1 is of great interest as a potential protein enabling resistance given the comparatively high expression in ECO-R-T (8.7) and its known activity on environmental toxins and xenobiotics.

TABLE 3
Differentially expressed genes involved in
xenobiotic detoxification expressed in ECO-R-T
compared to ECO-S-T 24-hr after quinclorac application.

| | | Fold |
|---|---|---|
| Gene Family | Gene | Change |
| Aldolase | Fructose-bisphosphate aldolase, chloroplastic | 4 |
| Amino- | Branched-chain-amino-acid aminotransferase | 4 |
| transferase | 5, chloroplastic | |
| | Aspartate aminotransferase, chloroplastic | 4 |
| | Alanine aminotransferase 2 | 4 |
| | Alanine aminotransferase 2 | 4 |
| | Alanine aminotransferase 2 | 4 |
| Amylase | Beta-amylase 1, chloroplastic | 4 |
| Cytochrome | CYP89A2 | −10 |
| P450 | CYP72A15 | −7 |
| | CYP71A9 | −6 |
| | CYP71A1 | 3 |
| | CYP71A1 | 3 |
| | CYP71A8 | 4 |
| | CYP71A1 | 5 |
| | CYP71A4 | 6 |
| | CYP709B2 | 6 |
| | CYP71A1 | 6 |
| | CYP709B2 | 7 |
| | CYP709B2 | 11 |
| GST | GSTU20 | 6 |
| GT | UGT74D1 | −4 |
| | UGT88A1 | 4 |
| | UGT73E1 | 5 |
| | UGT73D1 | 8 |
| | UGT75D1 | 9 |
| Hydrolase | Pyrimidine-specific ribonucleoside hydrolase RihA | −9 |
| | IAA-amino acid hydrolase ILR1-like 4 | −6 |
| | Probable xyloglucan endotrans- | −4 |
| | glucosylase/hydrolase protein 28 | |
| | Nudix hydrolase 21, chloroplastic | 3 |
| | Nudix hydrolase 21, chloroplastic | 4 |
| | Haloacid dehalogenase-like hydrolase | 4 |
| | domain-containing protein 3 | |
| Peptidase | Serine carboxypeptidase-like 42 | −5 |
| | Serine carboxypeptidase-like 42 | −5 |
| | Desumoylating isopeptidase 1 | 4 |
| | Prolyl endopeptidase | 8 |
| | Prolyl endopeptidase | 9 |
| | Prolyl endopeptidase | 15 |

### The Role of Constitutive Gene Induction in Evaluating Underlying Differences in ECO-S and ECO-R

Transcriptome characterization of the physiological status of ECO-S and ECO-R, without herbicide, and its response to quinclorac, is key in understanding the signal cascade and whole-plant response of E. colona to this auxin-mimic herbicide. In ECO-R-N compared with ECO-S—N, DNA transcription and protein synthesis, as well as the ethylene activated signaling pathways were enriched. The enriched ethylene pathway involves several ethylene response transcription factor genes. They are linked to abiotic stress response as well as sequence-specific binding to a pathogenesis promoter sequence. A higher abundance of gene transcripts associated with plant processes associated with carbon uptake assimilation and energy production were observed in ECO-R-N. This high level of activities would support any number of functions necessary for resistance. More importantly, the elevated activities prior to herbicide action would allow the plant to tolerate adverse conditions following treatment. Among the constitutively enhanced genes in ECO-R-N were several associated with trehalose biosynthesis. This sugar produced by these enzymes, has been extensively studied for its role in abiotic stress tolerance but not in herbicide resistance. The increase in the number of transcripts prior to herbicide treatment may be an indication of the predisposition of this accession to tolerate negative herbicide actions

Immediate action following quinclorac treatment in ECO-S is a stark contrast to ECO-R ECO-S signal cascade. Ethylene- and ABA-activated signaling pathways were significantly enriched following quinclorac treatment indicating endogenous ethylene and ABA production. This was validated by several ABA mediated genes including ASR1, a water stress tolerance gene, which is stimulated by ABA concentrations [51]. Nitrate transporter activity was enriched, suggesting that the demand for proteins is elevated in response to quinclorac. This response is also linked to endogenous ethylene build-up, which has a stimulatory effect on nitrate uptake and assimilation within the plant [52]. It is evident that feedback inhibition of the ACC synthase has occurred by 24 hours resulting in depression of the ACC synthase due to the elevated ethylene concentrations [53]. To catabolize the built-up ACC from the initial stimulation by ACC synthase, two ACC oxidase genes were induced which would lead to the high ethylene and toxic cyanide concentrations. Concomitantly the NECD enzyme was also repressed to limit ABA production. Auxin and ABA catabolismresponses were enriched to reduce the stimulatory effects of the exogenous auxin (quinclorac) response, and the endogenous ABA, respectively. ABA 8′-hydroxylase, was also induced to limit the concentration of ABA. ABA synthesis results in the closing of stomata, limiting water movement and gas exchange but also leads to the buildup of reactive oxygen species that cause tissue decay and senescence [54]. This was supported by several enriched processes related to anaerobic conditions and peroxidase activity, and further supported by the induction of RAP2-2. This gene is a transcriptional activator for the production of phytoene synthase and phytoene desaturase. Both enzymes are required for carotenoid biosynthesis, which would be necessary to mitigate the effects of excess energy build-up due to reduced electron flow resulting from stomatal closure. Collectively, these genes provide the underlying transcriptome response of E. colona following treatment. These are useful in describing the herbicide action in the plant and may provide a basis for evaluating other herbicide typically found in rice production systems.
ECO-R Signal Cascade. The gene expression profile for ECO-R-T was somewhat unexpected given the high level of resistance in this population. Most genes were repressed following treatment and gene ontology terms were not enriched. The gene expression profile indicated that the plant is repressing most processes following treatment and energy is expended on only a small number of genes/functions. The repressed pathways include photosynthetic, carbon assimilation, carbon metabolism, respiratory, and fatty acid synthesis pathways. Acetyl-CoA was induced following treatment indicating a buildup in fatty acid synthesis. The ACCase enzyme is the target for the cyhalofop herbicide, indicating a possible link between the responses to the two herbicides, especially when applied together. Of the few genes, which were induced by the treatment, those relating to the ethylene synthesis pathway, ACC synthase and ACC oxidase, were functioning. ACC synthase in particular was significantly induced following treatment indicating that the quinclorac reached its target. However, it does not appear given the comparison in responses between ECO-S-T and ECO-R-T that the downstream perception of the ethylene or ABA response is occurring. The general repression across most major gene families and in the functional transcripts indicates that the quinclorac-resistant plant averts lethal effects of the herbicide by limiting its biochemical output and entering a physiological ‘stasis’ state. This would mean not only a transient reduction in plant productivity but also a mitigation of the toxic production of cyanide and other harmful secondary effects.

### Trehalose Biosynthesis in ECO-R-N Plays a Significant Role in the Abiotic Stress Response by ECO-R

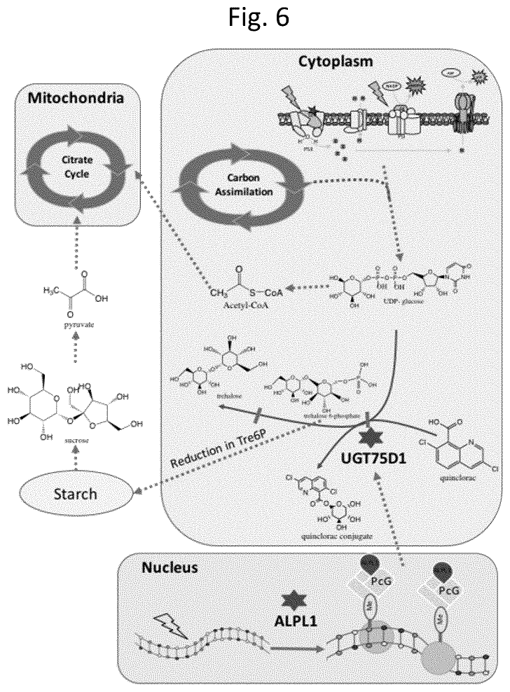

Several ontological terms and respective genes are presented in this research specifically related to trehalose metabolism in response to stress. Trehalose is a unique biological sugar which has been characterized as an important component in cellular metabolism and critical for proper plant growth and development [58,59]. Its role in rice abiotic stress tolerance has been investigated [60], but to date no research into herbicide activities have been described. This nonreducing sugar has several roles of interest to this research: its regulatory and signaling effect on sucrose, its role in membrane stability, and its ability to neutralize reactive oxygen species. The trehalose sugar and its precursor, intermediary, compound trehalose-6-P (Tre6P) both serve active roles in abiotic stress tolerance and may reveal a component in plant physiology that aids in herbicide resistance (FIG. 6). The presence of the abundant trehalose biosynthetic genes, which are highly expressed in ECO-R-N, suggest a buildup of free trehalose and Tre6P in the plant. Tre6P is an intercellular signal for starch to sucrose conversion and is a direct measure of sucrose concentrations in the plant [58]. The build-up of Tre6P would occur from the presence of the TPP enzymes in ECO-R-N, which would partition the carbon/sugar production toward starch synthesis [59]. Given the elevated photosynthetic and carbon related processes of the plant prior to treatment, it should more than supplement its need for carbon precursors and energy. Elevated TPP also has a synergistic effect on the photosynthetic capacity of the plant by signaling a higher demand for carbon which is the rate limiting step under high light intensity [61,62]. Following herbicide application, when the plant is responding by repressing the photosynthesis and carbon assimilation processes, the decrease in Tre6P imparted by the lack of carbon, would induce a starch to sucrose conversion. This presence of sucrose would then be available for the several critically induced processes that would need the carbon under the ‘stasis’ state exhibited by ECO-R-T. A second component of this trehalose build-up would be its role in the membrane stability following herbicide action including serving as a protectant against cyanide induced membrane decoupling, the production of ROS under high light intensity, or long-term water deprivation stress. The trehalose sugar is capable of forming hydrogen bonds with the hydrophobic head of the lipid bilayer, stabilizing it against oxidative and water deprivation stress, or potentially in the case of quinclorac against cyanide decoupling [63,64]. This will also stabilize membranes against destructive compounds such as free radicals and ROS. More importantly, trehalose has the ability scavenge both hydrogen peroxide and ROS, reducing the negative effects they may cause following herbicide action [65-67]. This would mitigate the destructive secondary or tertiary effects of the herbicide. This potential role for trehalose has not been described as a preventative measure against herbicide action nor has it been described in terms of herbicide response. This would require further investigation to validate the results, however, given the abundance of literature on the activities of trehalose under plant abiotic stress, there is potential for the role of this compound in co-evolutionary adaptation.

### Proposed Quinclorac Detoxification Mechanism

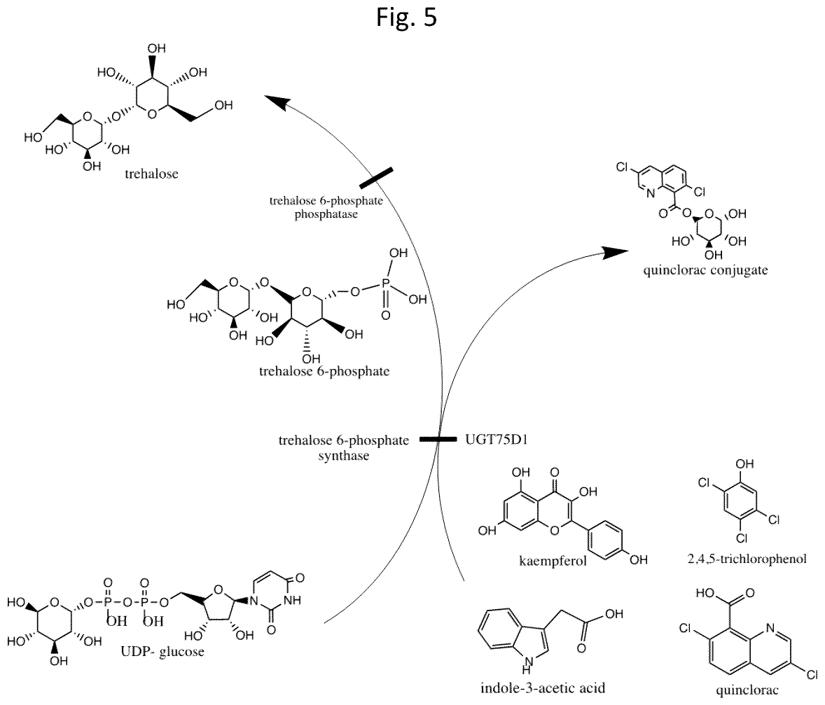

To investigate potential causal agents in resistance, the expression profile for ECO-R-T was surveyed for the known cyanide detoxification enzyme, β-cyanoanaline synthase, previously implicated in quinclorac resistance [23]. This enzyme was not identified amongst the response transcripts. UGT75D1 was induced and acts on the IAA molecule with UDP-glucose to form 1-O-indole acetyl glucose ester, it also has been investigated for its role in xenobiotic detoxification [47]. Specifically, through interaction with the carboxylic acid side chains. This enzyme has not been described as a metabolic enzyme for quinclorac but GT enzymes have been described as non-target-site resistance mechanisms specifically involved in phase II of xenobiotic detoxification [17]. Phase II GT activity requires the oxidation or hydrolysis of compounds to expose OH or NH2 for conjugation. The quinclorac molecule contains an exposed OH⁻ side group for which UGT75D1 can interact, suggesting the phase I step would not be necessary. UGT75D1 will bind to IAA but preferentially binds to endogenous kaempferol and exogenous 2,4,5-trichlorophenol, another pesticide [55]. Both of these compounds contain similar phenolic ring structures, OH side groups, and exposed chloride groups. This reaction would require a ready source of free UDP-glucose for which the GT could conjugate to the quinclorac molecule. The trehalose biosynthetic pathway would provide this to the system. The limiting of the pathway by the post-application physiological cascade in ECO-R-N would lead to a build-up of the UDP-glucose, as TPP is repressed. Given the elevated expression in ECO-R-T following treatment (7.3) and the comparatively high expression to ECO-S-T (8.7), UGT75D1 is the most probable enzyme responsible for degradation of quinclorac. The quinclorac conjugation involving the interconnected trehalose biosynthesis with the potential endogenous (IAA and kaempferol) and exogenous (2,4,5 trichlorphenol) compounds with affinity for UGT75D1 are presented in FIG. 5.

The driving mechanism behind the elevated UGT75D1 and several other stress responsive proteins is also an important consideration. Given the response pattern of ALPL1, it is clear that this protein provides some necessary function in the ECO-R plant response. Further investigation into this protein revealed its structural similarity to the ANTAGONIST OF LIKE HETEROCHROMATIN PROTEIN1 (ALP1), containing a unique harbinger transposase derived nuclease domain [38,56]. This domain allows for the targeting of specific regions of methylated DNA, including those being repressed by polycomb group proteins, which repress transcriptional activity. ALPI has been identified in association with several critical growing regions of the cell including a cis-acting factor that modulate physiological activities and result in pleiotropic effects [39]. Given the notable response and comparative expression levels, it is possible that ALPL1 is stimulated by the quinclorac induced stress and antagonizing a polycomb group residing upstream of the UGT75D1 protein, allowing for induction and elevated expression (FIG. 6).

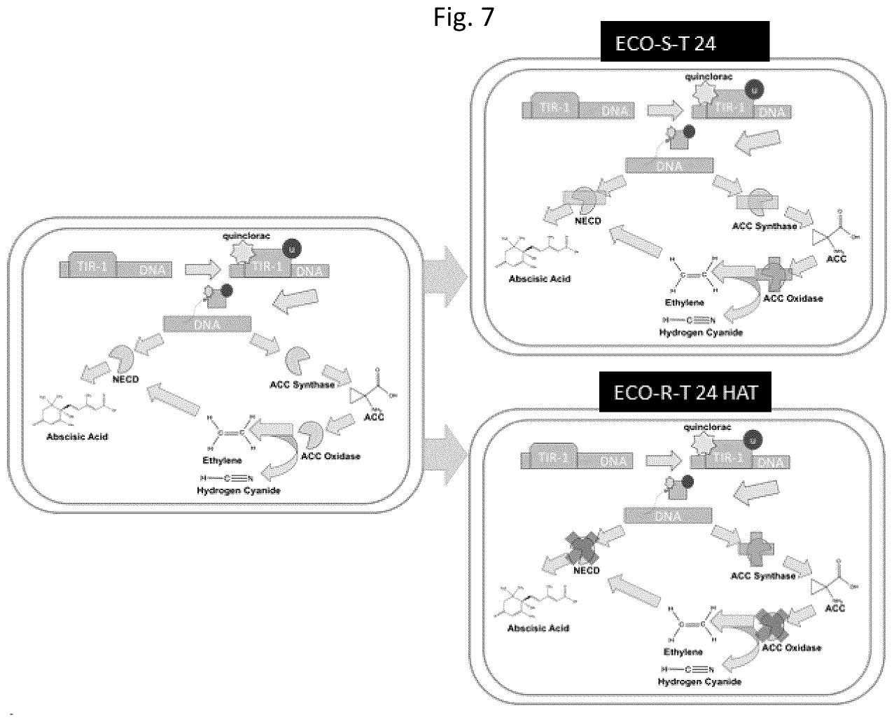

Quinclorac response and evolved herbicide resistance is a complex process involving multiple biological pathways. In the susceptible accession, this research validates the previous literature on quinclorac response and expands the description to further access the ABA mitigated responses. This research is able to conclude that the interaction of quinclorac with its target is rapid and the response occurs within 24 hours (FIG. 7). The necrosis and cell death, which occurs after this time is directly linked to this immediate activity. In response to the herbicide, E. colona enters an unstable stress response that results in the induction of several disease, abiotic, and metabolic related genes to reduce the impact of the herbicide. Several metabolism genes are induced with auxin hormone activity but their specificity and quantity does not appear to relieve the stress. ECO-R is a unique population with multiple-resistance and an extremely high level of quinclorac resistance. Without herbicide treatment, this population is well adapted to abiotic stress and is predisposed to tolerate a number of harsh conditions, including some herbicides give the enhanced gene set. The enrichment of the trehalose pathway has not been deeply investigated in weed species but appears to play a pivotal role in the evolved processes in this population. Not only would the presence of high trehalose concentrations aid in stress response and potentially mitigate the negative effects of the herbicide, but the presence of the pathway may aid the functioning of the potential glucosyltransferase resistance mechanism. Traditional RNA-sequencing analysis in weed science uses a R and S sample from the same population for characterization of the specific resistance mechanism, which is a shortcoming of our research [57]. However, by using the methodology described in this research we were able to better evaluate the herbicide response in a susceptible population and differentiate the underlying potential biological frameworks which contribute to the resistant phenotype. The results of this experiment and the proposed pathway need to be validated using biochemistry and molecular biology techniques. If validated, these results are the first characterized resistance mechanism that utilizes UGT75D1 for resistance and also has evolved an interconnected mechanism that would aid in general abiotic stress tolerance.

### Materials and Methods

### Plant Materials

Beginning in 2010, through the 2016 cropping cycle, Echinochloa spp. from throughout the rice producing counties of Arkansas, USA, located along the Mississippi River were sampled for a survey of the current status of herbicide resistance. Seeds were bulk sampled from plants that had matured in rice and soybean production fields, which had survived at least one herbicide application, were collected and sent to the University of Arkansas Altheimer Laboratory in Fayetteville for characterization and evaluation of herbicide resistance to common rice herbicides. Results from this screen can be found in Rouse et al. in which the method for characterization and results of the profiling are presented. From this screening program, two populations of E. colona were selected for use in this experiment. ECO-R is a multiple-resistant population from Lawrence County, Arkansas, characterized with resistance to three rice herbicides-cyhalofop, propanil, and quinclorac; as well as one soybean herbicide-glufosinate. This population has been further characterized with a high level of resistance to propanil (>8× field dose) and quinclorac (>32× field dose); the cyhalofop and glufosinate resistance is low comparatively (˜2× field dose) (data not shown). The second population, ECO-S, was selected as a susceptible standard, due to its similar cropping history and geographic location to ECO-R. ECO-S is characterized as susceptible to the aforementioned herbicides, however for propanil, tolerance is observed to approximately twice the recommended field dose. To establish inbred and homozygous accessions for the experiment, a single plant, verified as resistant/susceptible of ECO-R and ECO-S were grown in isolation to produce seed for further experiments. Due to it low outcrossing rate, a single generation was enough to achieve the desired genetic purity.

Pure-line generated seed of each accession were germinated in pots containing potting soil within a temperature/light controlled growth chamber set to a 14-hr day length, 33 C day temperature, and 24 C night time to simulate environmental conditions early in the rice growing season. A single plant was maintained in each of the pots and used for the treatments; two pots were used as individual biological replicates. At the two-leaf growth stage, in which two collars on the plant are visible approximately two weeks after planting, the plants were moved inside to an air propelled mechanized spray chamber for herbicide application; the sprayer was calibrated to deliver 187 L ha-1 using a 250-mL tank volume. Pots for each of the respective treatments were labeled as either ECO-R/S-T for the treated samples and ECO-R/S-N for the non-treated counterpart. To minimize the effect that the sprayer may play in the application, both plants of the ECO-R and ECO-S were treated at the same time. After approximately 30 minutes, allowing for the plants to dry, the treated and non-treated plants of both accessions were moved back into the growth chamber. Precisely 24-hours after application, the aboveground portion of each of the plants were removed and immediately frozen in liquid nitrogen to cease all biological function. Samples were then transferred to RNAlater™-ICE (Invitrogen, Carlsbad CA, USA) for shipment to the Clemson University Genomics Institute (CUGI), in Clemson, South Carolina.

### RNA Extraction, Processing, and Sequencing

RNA was extracted from the young leaf tissues of both replications for the ECO-R and ECO-S, T and N samples, at Clemson University. Total RNA was extracted with a kit according to the manufactures instructions. The extracted RNA was treated with DNase (Invitrogen, Calsbad, CA, USA) to remove any DNA contamination prior to further processing. The samples were prepared for sequencing by CUGI. For library preparation, the TruSeq Stranded Total RNA kit (Illumina Inc., San Diego, CA, USA) was used according to the instructions provided by the manufacturer to produce a paired-end library for sequencing. Ribosomal RNA was removed using target-specific oligonucleotides paired with rRNA removal beads, removing all cytoplasmic and mitochondrial rRNA that may result in poor quality results. RNA was fragmented and reverse transcribed to cDNA using random primers, followed by a second strand cDNA synthesis. Each fragment is then ligated with an additional ‘A’ and an adapter for sequencing. The PCR enriched product is then used to create the final cDNA library. All samples were sequenced on an Illumina Hiseq 2500 platform housed in the Holdings Cancer Center at the Medical University of South Carolina, Charleston, SC, USA. Samples, regardless of treatments or replication were ran across three lanes to reduce sequencing errors from the equipment. The resulting data were processed by CUGI.

### Transcriptome Assembly and Annotation

A de novo transcriptome was assembled from the treatments described for this experiment as well as several treatments, which included herbicides profiled in ECO-R. The treatments were applied to both ECO-R and ECO-S samples with two replications as described previously. In total, 20 individual plants, from both the ECO-R and ECO-S accessions, as well as T and NT samples, were used for the assembly. The transcriptome was assembled using the Trinity RNA-Seq pipeline (Broad Institute, Cambridge, MA, USA). Raw data were assessed for quality using FastQC (Babraham Institute, Cambridge, UK) and then processed to remove adapter sequences and low quality bases using a sliding window method [69]. The processed data were then rerun using FastQC to ensure high quality reads. Using the TrinityRNASeq 2.2.0 software, the samples were normalized, by replication, using a coverage size of 100 and kmer of 32. The normalized reads were then assembled as transcripts and genes using Trinity with the stranded library set as the default. Transdecoder 3.0.1 (Broad Institute) was used to scan the transcriptome for one open reading frame based on homology from the blastP database and to identify existing proteins using HMM Scan against pfam; transcripts matching both criteria were retained. CD-HIT-EST (Sanforn Burnham Prebys Medical Discovery Institute, San Diego, CA, USA) was used to cluster the transcripts based on sequence identity, sequences with 98% or greater similarity were retained. The transcriptome was assessed for transcriptome completeness using BUSCO (University of Geneva, Geneva, CH). Following assembly, the Trinotate 3.0 suite of software (trinotate) was used for functional annotation of the transcriptome via homology to BLAST+ and Swissprot databases to produce protein identification information based on HMMER and PFAM as well as generate information for the primary annotation databases including eggNOG, GO, and KEGG.

### Gene Ontology Analysis

Gene ontology enrichment/depletion analysis was used to describe the functional components associated with herbicide response and resistance. Using the Trinotate output, the goseq package from Bioconductor was used to assign GO terms to the transcripts from the transcriptome. The analysis of enrichment/depletion was performed on transcripts which had been expressed or depressed at a log 2 fold-change of ≤−2 or ≥2 and a p-value of ≤0.01. The results of the analysis were visually assessed using REVIGO (revigo) to best characterize the resulting ontological terms and describe interconnected pathways within the treatments. For description of the gene ontology terms and functions of the terms EggNOG and GO Consortium databases.

### Differential Gene Expression

Using the de novo transcriptome as a reference, differential gene expression was quantified by comparing several pairwise orthogonal sets of treatments. The multi-dimensional scaling (MDS) plot was generated to assess the disparity of the replications for each treatment and accession. The second replication for ECO-R-N was excluded, as it did not fit within an acceptable distance on the MDS plot to the other samples used for the analysis, all other treatments were retained for analysis. A GTF file of the transcripts was generated as a boundary for comparing each sample to the reference transcriptome. Feature counts were generated using the Subread package (subread), allowing for quantification of the differentially expressed transcripts with each replication, which were paired concordantly. The Bioconductor (bioconductor) package-edgeR, developed for use within the R statistical software program (r-project), was used to quantify the filtered raw counts produced from the RNA-sequencing [72,73]. Standard normalization using trimmed mean of M-values (TMM) was applied to the counts. The counts were fit using a GLM model for determination of significance (p≤0.01) and a likelihood ratio tests (LRT) for specific comparisons of interest in the experiment. The resulting analysis was then evaluated using a false discovery rate for p-value correction to reduce the error in the results. Volcano plots, for visual assessment of gene expression, and a table of log 2-fold changes with respective genes within the comparisons of interest were generated from the analysis. These results were then used in subsequent descriptive analysis to describe the patterns of expression within the tested conditions. In order to reduce the number of potential genes used in describing the expression patterns, categories or groupings were assigned to the sets of differentially expressed genes. Based on a review of the literature, enzymes which may be involved in one of the four phases of chemical detoxification were assigned into one of eight categories: ABC transporters, aminotrasferases, amylase, cytochrome P450s, glutathione-S-transferases, glucosyltransferase, hydrolases, and peptidases [18,74,75]. The description of the genes and pathways are based on the data on the Uniprot and KEGG databases [77].

### REFERENCES FOR EXAMPLE 1

1. Chauhan B S, Jabran K, Mahajan G. Rice Production Worldwide. Cham, Switzerland: Springer; 2017.
2. Valverde B E, Riches C R, Caseley J C. Prevention and management of herbicide resistant weeds in rice: Experiences from Central America with Echinochloa colona. 2000.
3. GBIF Secretariat. GBIF Backbone Taxonomy [Internet]. 2013 [cited 1 Apr. 2014]. doi: 10.15468/390MEI
4. Burgos N R, Rouse C E, Tseng™, Abugho S B, Hussain T, Salas R A, et al. Resistance Profiles of Echinochloa colona in Arkansas. 68th Southern Weed Science Society Annual Meeting. Savannah, GA: Southern Weed Science Society; 2015. p. 22.
5. Barrett S H. Crop mimicry in weeds. Econ Bot. 1983; 37:255-282. doi: 10.1007/BF02858881
6. Yang X, Fuller D Q, Huan X, Perry L, Li Q, Li Z, et al. Barnyard grasses were processed with rice around 10000 years ago. Sci Rep. Nature Publishing Group; 2015; 5:16251. doi: 10.1038/srep16251
7. Anonymous. Plants Profile for Echinochloa (cockspur grass). In: USDA Natural Resources Conservation Service [Internet]. [cited 3 Jul. 2017].
8. Van Wychen L. 2015 Baseline Survey of Most Common and Troublesome Weeds in the United States and Canada. In: Weed Science Society of American National Weed Survey Dataset [Internet]. 2015 [cited 22 Mar. 2017].
9. Smith R J. Competition of barnyardgrass with rice cultivars. 1974; 22:423-426 . . .
10. Smith R J. Weed Competition in Rice. Weed Sci. 1968; 16:252-255.
11. Workman D. Rice Exports by Country [Internet]. 2017 [cited 27 Jun. 2017].
12. Talbert R E, Burgos N R. History and Management of Herbicide-resistant Barnyardgrass (Echinochloa Crus-galli) in Arkansas Rice. Weed Technol. 2007; 21:324-331. doi: 10.1614/WT-06-084.1
13. Cobb A H, Reade J P. Auxin Type Herbicides. Herbicides and Plant Physiology. Second. West Sussex, United Kindom: Wiley-Blackwell; 2010. pp. 133-156.
14. Grossmann K. Auxin herbicides: Current status of mechanism and mode of action. Pest Manag Sci. 2010; 66:113-120. doi: 10.1002/ps.1860
15 Grossmann K, Scheltrup F. Selective Induction of 1-Aminocyclopropane-1-carboxylic Acid (ACC) Synthase Activity Is Involved in the Selectivity of the Auxin Herbicide Quinclorac between Barnyard Grass and Rice. Pestic Biochem Physiol. 1997; 58:145-153. doi: 10.1006/pest.1997.2290
16. Anonymous. Herbicide Resistance and Herbicide Tolerance Defined. Weed Technol. 1998; 12:789. doi:
17. Délye C. Unravelling the genetic bases of non-target-site-based resistance (NTSR) to herbicides: A major challenge for weed science in the forthcoming decade. Pest Manag Sci. 2013; 69:176-187. doi: 10.1002/ps.3318
18. Kreuz K, Tommasini R, Martinoia E. Old Enzymes for a New Job. Plant Physiol. 1996; 349-353.
19. Riar D S, Norsworthy J K, Bond J a., Bararpour M T, Wilson M J, Scott R C. Resistance of Echinochloa crus-galli Populations to Acetolactate Synthase-Inhibiting Herbicides. Int J Agron. 2012; 2012:1-8. doi: 10.1155/2012/893953
20. Alarcón-Reverte R, García A, Urzúa J, Fischer A J. Resistance to Glyphosate in Junglerice (Echinochloa colona) from California. Weed Sci. 2013; 61:48-54. doi: 10.1614/WS-D-12-00073.1
21. Lopez-Martinez N, Marshall G, De Prado R. Resistance of barnyardgrass (Echinochloa crus-galli) to atrazine and quinclorac. Pestic Sci. 1997; 51:171-175. doi: 10.1002/(SICI) 1096-9063 (199710) 51: 2<171::AID-PS612>3.0.CO; 2-7
22. Hoagland R E, Graf G, Handel E D. Hydrolysis of 3,4-dichloropropionanilide by plant aryl acylamidases. Weed Res. 1974; 14:371-374. doi: 10.1111/j. 1365-3180.1974.tb01077.x
23. Yasuor H, Milan M, Eckert J W, Fischer A J. Quinclorac resistance: A concerted hormonal and enzymatic effort in Echinochloa phyllopogon. Pest Manag Sci. 2012; 68:108-115. doi: 10.1002/ps.2230
24. Abe H, Yamaguchi-Shinozaki K, Urao T, Iwasaki T, Hosokawa D, Shinozaki K. Role of arabidopsis MYC and MYB homologs in drought- and abscisic acid-regulated gene expression. Plant Cell. 1997; 9:1859-1868. doi: 10.1105/tpc.9.10.1859 [doi] \r9/10/1859 [pii]
25. Ohme-takagi M, Shinshi H. Ethylene-inducible DNA binding proteins hthat interact with an ethylene-responsive element. 1995; 7:173-182.
26. Song C-P, Agarwal M, Ohta M, Guo Y, Halfter U, Wang P. Role of an Arabidopsis AP2/EREBP-Type Transcriptional Repressor in Abscisic Acid and Drought Stress Responses. Plant Cell. 2005; 17:2384-2396. doi: 10.1105/tpc.105.033043.ABI2
27. Solano R, Stepanova A, Chao Q, Ecker J R. Nuclear events in ethylene signaling a transcriptional cascade mediated by ETHYLENE-Insensitive2 and ETHYLEN-RESONSE-FACTOR1. Genes Dev. 1998; 12:3703-3714. doi: 10.1101/gad. 12.23.3703
28. Hong Z, Ueguchi-Tanaka M, Umemura K, Uozu S, Fujioka S, Takatsuto S, et al. A Rice Brassinosteroid-Deficient Mutant, ebisu dwarf (d2), Is Caused by a Loss of Function of a New Member of Cytochrome P450 Zhi. Plant Cell. 2003; 15:2900-2910. doi: 10.1105/tpc.014712.grass
29. Kandel S, Sauveplane V, Compagnon V, Franke R, Millet Y, Schreiber L, et al. Characterization of a methyl jasmonate and wounding-responsive cytochrome P450 of Arabidopsis thaliana catalyzing dicarboxylic fatty acid formation in vitro. FEBS J. 2007; 274:5116-5127. doi: 10.1111/j.1742-4658.2007.06032.x
30. Gandia-Herrero F, Lorenz A, Larson T, Graham I A, Bowles D J, Rylott E L, et al. Detoxification of the explosive 2,4,6-trinitrotoluene in Arabidopsis: Discovery of bifunctional O- and C-glucosyltransferases. Plant J. 2008; 56:963-974. doi: 10.1111/j.1365-313 X.2008.03653.x
31. Katsir L, Davies K A, Bergmann D C, Laux T. Peptide signaling in plant development. Curr Biol. Elsevier Ltd; 2011; 21: R356-R364. doi: 10.1016/j.cub.2011.03.012
32. Hauser F, Waadt R, Schroeder J I. Evolution of abscisic acid synthesis and signaling mechanisms. Curr Biol. Elsevier Ltd; 2011; 21: R346-R355. doi: 10.1016/j.cub.2011.03.015
33. Leyser O. Auxin, self-organisation, and the colonial nature of plants. Curr Biol. Elsevier Ltd; 2011; 21: R331-R337. doi: 10.1016/j.cub.2011.02.031
34 Downton W, Loveys B R, Grant W J R. Stomatal closure fully accounts for the inhibition of photosynthesis by abscisic acid. New Phytol. 1988; 108:263-266. doi: 10.1111/j. 1469-8137.1988.tb04161.x
35. Santiago J, Rodrigues A, Saez A, Rubio S, Antoni R, Dupeux F, et al. Modulation of drought resistance by the abscisic acid receptor PYL5 through inhibition of clade A PP2Cs. Plant J. 2009; 60:575-588. doi: 10.1111/j. 1365-313 X.2009.03981.x
36. Tuteja N, Mahajan S. Calcium signaling network in plants: an overview. Plant Signal Behav. 2007; 2:79-85. doi: 10.4161/psb.2.2.4176
37. Kobayashi M, Ohura I, Kawakita K, Yokota N, Fujiwara M, Shimamoto K, et al. Calcium-Dependent Protein Kinases Regulate the Production of Reactive Oxygen Species by Potato NADPH Oxidase. Plant Cell Online. 2007; 19:1065-1080. doi: 10.1105/tpc.106.048884
38. Liang S C, Hartwig B, Perera P, Mora-García S, de Leau E, Thornton H, et al. Kicking against the PRCs-A Domesticated Transposase Antagonises Silencing Mediated by Polycomb Group Proteins and Is an Accessory Component of Polycomb Repressive Complex 2. PLOS Genet. 2015; 11:1-26. doi: 10.1371/journal.pgen.1005660
39. Ricci W A, Zhang X. Public Service by a Selfish Gene: A Domesticated Transposase Antagonizes Polycomb Function. PLOS Genet. 2016; 12:10-12. doi: 10.1371/journal.pgen.1006014
40. Supek F, Bošnjak M, Škunca N, Šmuc T. Revigo summarizes and visualizes long lists of gene ontology terms. PLOS One. 2011; 6. doi: 10.1371/journal.pone.0021800
41. Alonso J M, Hirayama T, Roman G, Nourizadeh S. EIN2, a Bifunctional Transducer of Ethylene and Stress Responses in Arabidopsis. Science (80-). 1999; 284:2148-2152. doi: 10.1126/science.284.5423.2148
42. Welsch R, Maass D, Voegel T, DellaPenna D, Beyer P. Transcription Factor RAP2.2 and Its Interacting Partner SINAT2: Stable Elements in the Carotenogenesis of Arabidopsis Leaves. Plant Physiol. 2007; 145:1073-1085. doi: 10.1104/U.S. Plant Pat. No. 107,104828
43. Lim E K, Doucet C J, Li Y, Elias L, Worrall D, Spencer S P, et al. The activity of Arabidopsis glycosyltransferases toward salicylic acid, 4-hydroxybenzoic acid, and other benzoates. J Biol Chem. 2002; 277:586-592. doi: 10.1074/jbc.M109287200
44. Tognetti V B, Van Aken O, Morreel K, Vandenbroucke K, van de Cotte B, De Clercq I, et al. Perturbation of Indole-3-Butyric Acid Homeostasis by the UDP-Glucosyltransferase UGT74E2 Modulates Arabidopsis Architecture and Water Stress Tolerance. Plant Cell. 2010; 22:2660-2679. doi: 10.1105/tpc.109.071316
45. Levine A, Tenhaken R, Dixon R, Lamb C. H₂O₂ from the oxidative burst orchestrates the plant hypersensitive disease resistance response. Cell. 1994; 79:583-593.
46. Iusem N D, Bartholomew D M, Hitz W D, Scolnik P A. Tomato (Lycopersicon esculentum) transcript induced by water deficit and ripening. Trends Biochem Sci. 1993; 102:1353-1354.
47. Jackson R G, Lim E K, Li Y, Kowalczyk M, Sandberg G, Hogget J, et al. Identification and Biochemical Characterization of an Arabidopsis Indole-3-acetic Acid Glucosyltransferase. J Biol Chem. 2001; 276:4350-4356. doi: 10.1074/jbc.M006185200
48. Umemoto N, Kobayashi O, Ishizaki-Nishizawa O, Toguri T. cDNAs sequences encoding cytochrome P450 (CYP71 family) from eggplant seedlings. FEBS Lett. 1993; 330:169-173. doi: 10.1016/0014-5793 (93) 80266-W
49. O′keefe DP, Leto K J. Cytochrome P-450 from the Mesocarp of Avocado (Persea americana). Plant Physiol. 1989; 89:1141-1149. doi: 10.1104/pp. 89.4.1141
50. Mao G, Seebeck T, Schrenker D, Yu O. CYP709B3, a cytochrome P450 monooxygenase gene involved in salt tolerance in Arabidopsis thaliana. BMC Plant Biol. 2013; 13:169. doi: 10.1186/1471-2229-13-169
51. Fischer I, Camus-Kulandaivelu L, Allal F, Stephan W. Adaptation to drought in two wild tomato species: The evolution of the Asr gene family. New Phytol. 2011; 190:1032-1044. doi: 10.1111/j.1469-8137.2011.03648.x
52. Sugiyama T, Sakabira H. Regulation of carbon and nitrogen assimilation through gene expression. In: Foyer C, Noctor G, editors. Photosynthetic Nitrogen Assimilation and Associated Carbon and Respiratory Metabolism. Netherlands: Kluwer Academic Publishers; 2003. pp. 227-238.
53. Yang S F, Hoffman N E. Ethylene Biosynthesis and Its Regulation in Higher Plants. Annu Rev Plant Physiol. 1984; 35:155-189.
54. Grossmann K, Hansen H. Ethylene-triggered abscisic acid: A principle in plant growth regulation? Physiol Plant. 2001; 113:9-14.
55. Messner B, Thulke O, Schaffner A R. Arabidopsis glucosyltransferases with activities toward both endogenous and xenobiotic substrates. Planta. 2003; 217:138-146. doi: 10.1007/s00425-002-0969-0
56. Duan C-G, Wang X, Xie S, Pan L, Miki D, Tang K, et al. A pair of transposon-derived proteins function in a histone acetyltransferase complex for active DNA demethylation. Cell Res. Nature Publishing Group; 2017; 27:226-240. doi: 10.1038/cr.2016.147
57. Gaines T A, Lorentz L, Figge A, Herrmann J, Maiwald F, Ott M C, et al. RNA-Seq transcriptome analysis to identify genes involved in metabolism-based diclofop resistance in Lolium rigidum. Plant J. 2014; 78:865-876. doi: 10.1111/tpj.12514
58. Lunn J E, Delorge I, Figueroa C M, Van Dijck P, Stitt M. Trehalose metabolism in plants. Plant J. 2014; 79:544-567. doi: 10.1111/tpj.12509
59. Schluepmann H, Pellny T, van Dijken A, Smeekens S, Paul M. Trehalose 6-phosphate is indispensable for carbohydrate utilization and growth in Arabidopsis thaliana. Proc Natl Acad Sci USA. 2003; 100:6849-6854. doi: 10.1073/pnas.1132018100
60. Garg A K, Kim J-K, Owens T G, Ranwala A P, Choi Y D, Kochian L V., et al. Trehalose accumulation in rice plants confers high tolerance levels to different abiotic stresses. Proc Natl Acad Sci. 2002; 99:15898-15903. doi: 10.1073/pnas.252637799
61. Fernandez O, Béthencourt L, Quero A, Sangwan R S, Clément Christophe C. Trehalose and plant stress responses: Friend or foe? Trends Plant Sci. 2010; 15:409-417. doi: 10.1016/j.tplants.2010.04.004
62. Paul M, Pellny T, Goddijn O. Enhancing photosynthesis with sugar signals. Trends Plant Sci. 2001; 6:197-200. doi: 10.1016/S1360-1385 (01) 01920-3
63. Crowe J J, Crowe L M, Chapman D. Preservation of Membranes in Anhydrobiotic Organisms: The Role of Trehalose. Science (80-). 1984; 223:701-703.
64. Crowe J H, Hoekstra F A, Crowe L M. Anhydrobiosis. Annu Rev Physiol. 1992; 54:579-599.
65. Ali Q, Ashraf M. Induction of drought tolerance in maize (Zea mays L.) due to exogenous application of trehalose: Growth, Photosynthesis, Water Relations and Oxidative Defence Mechanism. J Agron Crop Sci. 2011; 197:258-271. doi: 10.1111/j.1439-037 X.2010.00463.x
66. Benaroudj N, Lee D H, Goldberg A L. Trehalose Accumulation during Cellular Stress Protects Cells and Cellular Proteins from Damage by Oxygen Radicals. J Biol Chem. 2001; 276:24261-24267. doi: 10.1074/jbc.M101487200
67. Luo Y, Li W M, Wang W. Trehalose: Protector of antioxidant enzymes or reactive oxygen species scavenger under heat stress? Environ Exp Bot. 2008; 63:378-384. doi: 10.1016/j.envexpbot.2007.11.016
68. Rouse C E, Burgos N R, Norsworthy J K, Tseng™, Starkey C. Echinochloa resistance to herbicides continues to increase in Arkansas rice fields. Weed Technol. 2017; In Press.
69. Bolger A, Lohse M, Usade B. Trimmomatic: A flexable read trimming tool for Illumina NGS data. Bioinformatics. 2015; btu170.
70. Huerta-Cepas J, Szklarczyk D, Forslund K, Cook H, Heller D, Walter M C, et al. EGGNOG 4.5: A hierarchical orthology framework with improved functional annotations for eukaryotic, prokaryotic and viral sequences. Nucleic Acids Res. 2016; 44: D286-D293. doi: 10.1093/nar/gkv1248
71. Ashburner M, Ball C A, Blake J A, Botstein D, Butler H, Cherry J M, et al. Gene Ontology: Tool for The Unification of Biology. Nat Genet. 2000; 25:25-29. doi: 10.1038/75556
72. Robinson M D, McCarthy D J, Smyth G K. edgeR: A Bioconductor package for differential expression analysis of digital gene expression data. Bioinformatics. 2009; 26:139-140. doi: 10.1093/bioinformatics/btp616
73. McCarthy D J, Chen Y, Smyth G K. Differential expression analysis of multifactor RNA-Seq experiments with respect to biological variation. Nucleic Acids Res. 2012; 40:4288-4297. doi: 10.1093/nar/gks042
74. Duhoux A, Carrere S, Duhoux A, Delye C. Transcriptional markers enable identification of rye-grass (Lolium sp.) plants with non-target-site-based resistance to herbicides inhibiting acetolactate-synthase. Plant Sci. Elsevier Ireland Ltd; 2017; 257:22-36. doi: 10.1016/j.plantsci.2017.01.009
75. Gardin J A C, Gouzy J, Carrère S, Délye C. ALOMYbase, a resource to investigate non-target-site-based resistance to herbicides inhibiting acetolactate-synthase (ALS) in the major grass weed Alopecurus myosuroides (black-grass). BMC Genomics. BMC Genomics; 2015; 16:590. doi: 10.1186/s12864-015-1804-x
76. UniProt: the universal protein knowledgebase. Nucleic Acids Res. 2017; 45: D158-D169. doi: 10.1093/nar/gkw1152
77. Kanehisa M, Furumichi M, Tanabe M, Sato Y, Morishima K. KEGG: New perspectives on genomes, pathways, diseases and drugs. Nucleic Acids Res. 2017; 45: D353-D361. doi: 10.1093/nar/gkw1092

### Example 2—Concerted Action of Abiotic Stress Responsive Genes May Impart High Resistance to Propanil in Multiple-Resistant Echinochloa colona

Propanil is amongst the oldest herbicide compounds used for selective control of Echinochloa spp. in rice production in the Midsouth, USA. Previous research into a multiple-resistant E. colona (ECO-R) population from a rice field in Arkansas was conducted. This research identified that an unknown metabolic enzyme is potentially allowing for high levels of resistance to propanil in this population. Physiological assays were able to conclude that the pattern of response was similar to inactivation of the propanil molecule by the aryl acylamidase enzyme, but further investigation was required. An RNA-sequencing experiment was conducted on the ECO-R population and a susceptible counterpart (ECO-S), to describe the response patterns following propanil treatment and elucidate the potential herbicide resistance mechanism of ECO-R based on differential gene expression. Using the de novo transcriptome produced by our research group previously, differential gene expression in ECO-S following propanil treatment indicates that 1765 genes were repressed and 1,775 were induced. In general, the stress response elucidated by ECO-S indicates perception of both abiotic and biotic stressors leading to the induction of abscisic acid and jasmonic acid metabolism. Several glucosinolate producing enzymes and hypersensitive response enzymes related to diseases were also induced. The propanil application induced trehalose biosynthesis. For ECO-R following propanil treatment, only 152 genes were induced but a number of similar processes including both abiotic and biotic stress perception were the same as ECO-S. The differential gene expression analysis revealed two cytochrome P450 enzymes-CYP709B2 (>8-fold induction) and CYP72A14 (˜3-fold induction) that have the potential to hydroxylate the propanil molecule in phase one degradation. The profile also shows induction of several glutathione-S-transferase and glycosyltransferase genes that may be involved in phase II conjugation of the 3,4-dichloroanaline and propionic acid molecules. This is the first such characterization of abiotic and biotic signal perception following propanil application using the transcriptome of multiple-resistant E colona.

Echinochloa spp. are weeds of global importance and widespread influence through the upland and lowland agricultural production systems of the world [1,2]. Current research in Arkansas, and throughout the US mid-south production regions, positions the Echinochloa genus as the number one most common and troublesome weeds impacting rice production and among the top ten in soybean and cotton production [3]. Its sphere of influence is largely due to its biology and morphology which allows for significant adaptive evolution under imposed stress in the agriculture landscape [4]. This adaptability under diverse agricultural systems may be indicative of its early co-domestication with rice as a millet crop over 10,0000 years ago, and may have had long term implications for its ability to mimic rice today [5,6]. Within US rice production, Echinochloa crus-galli, has historically been among the top most researched species. First characterized in Arkansas rice in 1968, it has since become a dominate factor in reducing crop yields in rice, second only to weedy rice in terms of threat to productivity [7,8]. One plant per square meter imposing season-long interference can reduce yield as much as 65 kg ha-1 and competition of approximately 50 plants in 0.1 m2 up to 37 days is can reduce rice crop yields by 20% [7,9]. While Echinochloa crus-galli has been widely accepted in the literature as the major species of importance, recent research indicates that E. colona is the primary species impacting Arkansas rice producers, and that complexes of E. colona, E. muricata, and E. crus-galli co-exist within single rice fields [10]. Throughout the southern US, Echinochloa spp. have been widely misidentified. Recent research indicates E. colona are the more dominate species throughout southern production fields and complexes of E. colona and E. crus-galli are present throughout most of Arkansas [12]. Due to this fact, we considered that both the E. colona and E. crus-galli have been colloquially referred to as barnyardgrass in the literature and thus are presented as Echinochloa spp. for data prior to 2017 in the US.

Herbicides have been a long-standing component of Echinochloa spp. management in rice and other cropping systems. In 1959, propanil was the among first commercially available herbicides for selective control of Echinochloa in rice [13]. As innovations continued through the 1990s, several other herbicides including quinclorac, fenoxaprop, clomazone, and cyhalofop were introduced. Later in the early 2000s, imidazolinone herbicides with the Clearfield rice System® were registered for use in rice. Propanil is a photosystem II (PSII) inhibitor (WSSA group 7), which irreversibly binds to the D1 protein blocking the interaction between plastoquinone and PSII, blocking electron flow through the complex [14]. The limitation in photosynthetic activity leads to reductions in carbon assimilation but the free energy build-up leads to the secondary effects of herbicide action, which is reactive oxygen species (ROS) and hydrogen peroxide production. These highly reactive molecules are capable of destroying cells. Rice is highly tolerant to propanil because of an elevated production of hydroxylating enzyme, aryl acylamidase, which is capable of detoxifying propanil into two metabolites: 3,4 dichloroanaline and propionic acid [15,16]. Due to the overreliance on propanil, resistant Echinochloa spp. have become a widespread problem. First documented in E. crus-galli in 1986 from populations in Greece, propanil-resistant Echinochloa spp. have evolved in 14 countries across the globe [17]. In the US-Midsouth, all of the states that make up the region-Arkansas, Mississippi, Missouri, Louisiana, and Texas contain resistant populations. In Arkansas, 50% of the Echinochloa spp. populations are resistant to propanil, while 12% of all populations are multiple resistant to propanil and quinclorac [10]. This is a major concern considering the US Midsouth alone accounts for 65% of the US rice production, leaving producers with concerns for the role resistance plays in crop management [18].

Herbicide resistance is an evolved survival trait as a consequence of sustained herbicide selection pressure, especially under prolonged mono-cropping systems with a lack of herbicide diversity or weed control methods [19]. Mechanisms of resistance are broadly categorized as target-site (TSR) or non-target-site (NTSR). TSR involves structural modification of the herbicide target protein, lowering the binding efficiency of the herbicide, and consequently reducing its efficacy [20]. These monogenic changes can be selected by continuous high-dose selection, eventually causing a shift toward a resistant population [19]. NTSR is a more complex polygenic mechanism that involves a network of abiotic stress response mechanisms that attempt to reduce the uptake of, modify, or redistribute the herbicide, to limit its availability at the site of action [22]. These processes include several phases of the xenobiotic detoxification process, employed by plants to mitigate the harmful effects of exogenous compounds [23]. This mechanism evolves slowly and often results from low dose selection over the course of several years; specifically, as the plant accumulates the necessary genetic components to persist through herbicide action. Echinochloa spp. have evolved resistance to seven herbicide modes of action involving both TSR and NTSR mechanisms [17]. TSR to acetolactate synthase-inhibiting herbicides [24], atrazine [25], and glyphosate [26,27] has been identified in Echinochloa spp. around the world. NTSR has also been identified involving several herbicide modes of action including clomazone [13,28], fenoxaprop and quinclorac [30]. Resistance to propanil in Echinochloa spp.is also a NTSR mechanism which involves detoxification via the aryl acylamidase, the same enzyme employed by rice [16, 31-33].

To evaluate the underlying causes of herbicide resistance, researchers have historically used physiological, biochemical, or molecule biology approaches. Until recently, genomic approaches were limited by lack of resources to investigate non-model organisms and a lack of genomic assemblies for comparative analysis [34,35]. Recently, several transcriptomes have been published for weedy species including one for E. crus-galli [36] and E. colona [37]. Results from these experiments have produced repositories of genes for further research and led to the identification of novel resistance mechanisms and biological pathways involved in herbicide response. Using genomics to assess herbicide resistance evolution in weedy species has led to the identification of several herbicide target genes [38], advanced phylogenetic analysis of herbicide targets, and identification of previously unknown herbicide detoxifying genes [39]. This research is considered novel to the weed science discipline. It presents new information that may be of value in the future of weed management and understanding weed biology.

Propanil resistance in Echinochloa spp. is a long-studied topic, but the recent research on the distribution of resistance in Arkansas is alarming. Using RNA-sequencing of a multiple-resistant Echinochloa colona, we utilized the transcriptome to describe the patterns of gene expression following propanil treatment and identify candidate genes involved in propanil resistance. To date, the definitive enzyme endowing resistance has been the aryl acylamidase protein, which has activities in both rice and Echinochloa spp. This research identifies two cytochrome P450 enzymes, capable of detoxifying propanil, in the absence of an elevated aryl acylamidase gene.

### Results

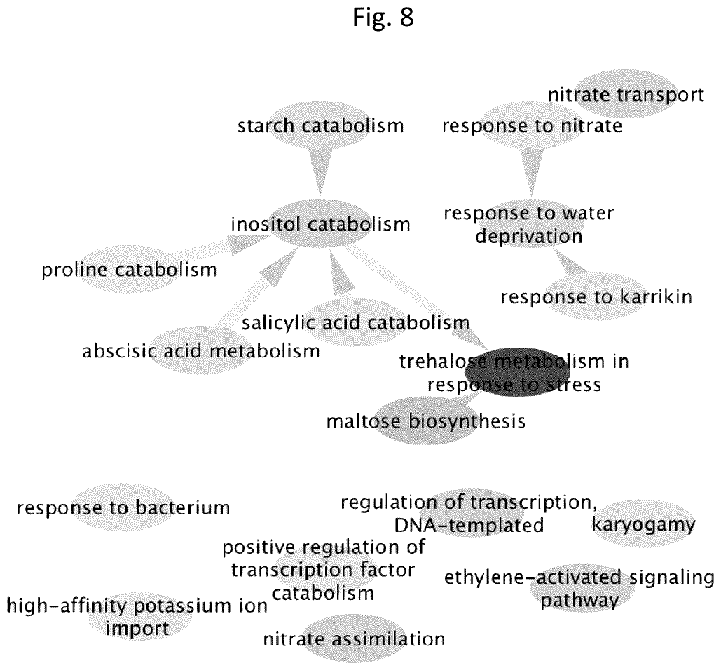

Biological Framework and Gene Expression Response to Propanil Action in Susceptible E. colona Populations
Gene network analysis reveals complex stress induced responses. The susceptible accession, ECO-S-T, expresses a series of abiotic stress proteins following treatment. Gene ontology or biological network characterization revealed several biological processes centered around inositol catabolismthat were enriched in ECO-S-T (FIG. 8). The sub-cluster-trehalose metabolism was enriched, which is a general plant response to stress. The ‘abscisic acid (ABA) metabolism’ supercluster, which has a role in stomatal closure and stress response signaling, was enriched. This contains several ontological terms including ‘ABA metabolic process’, ‘ABA catabolic process’, and ‘ABA-activated signaling’. Starch and salicylic acid catabolic processes were also significantly enriched. This is related to the ‘responses to water deprivation’ and ‘nitrate transport and assimilation’ terms that were also enriched. The remaining biological processes were involved in ethylene-activated signaling and DNA transcription regulation, including ‘transcription factor catabolism’. ‘Inositol oxygenase activity’, ‘ABA hydroxylase activity’, and ‘trehalose metabolism’ enzyme activities were all significantly enriched.
Growth regulation and maintenance genes are impacted by propanil. Twenty-four hours following treatment with propanil, 3,539 transcripts were differentially expressed; 1765 were repressed and 1771 were induced (Table 4). Several genes associated with photosynthesis were repressed including ATP synthase alpha subunits and plastocyanin, both of which are critical in the electron transfer process. However, three forms of ferredoxin-6 were induced 3.8- to 4.8-fold. In the mitochondria, the majority of transcripts were repressed 24 hours following propanil treatment including several ribosomal proteins, ATP synthase subunits, ADP/ATP carrier proteins, and many transferase type proteins. A vast number of DNA and RNA polymerase-related transcripts were repressed by as much as −8.7-fold. A similar number of proteins associated with DNA were enhanced, 45 of which were transcription factors, 8 chaperone proteins, and several other DNA binding proteins. Key genes relevant to carbon metabolism, phosphoenolypyruvate carboxylase (−2.1 to −3.4), and pyruvate dehydrogenase (−5.6 to −6.2) were also repressed. These are essential to the breakdown and utilization of stored energy reserves and the assimilation of new carbon products. Acetyl-CoA carboxylase (ACCase) and ACCase 2 were repressed up to −6.9-fold, indicating reduced fatty acid production. In terms of biological and physiological processes, a large number of genes related to sugar metabolism were induced by propanil treatment. Three sugar metabolism genes were repressed including those coding for proteins associated with sugar transport, indicating reduced, or no transmembrane movement. The vast majority of the induced genes (1.5- to 7.4-fold), were transcripts coding for various forms of enzymes in the trehalose biosynthetic pathway, including both the trehalose phosphate synthase (TPS) and trehalose-phosphate phosphatase (TPP) enzymes. The sugar transporter ERD6-like 6 was induced 2.3-fold, supporting the elevated status of trehalose synthesis, which would need to be transported across membranes to fulfill demand in various organelles and tissues.

TABLE 4
Summary of the repression and induction of
genes from the differential gene expression
analysis of each of the comparisons of interest.

| | | ECO-S-NT | ECO-S-NT | ECO-R-NT | ECO-S-T |
|---|---|---|---|---|---|
| | | vs | vs | vs | vs |
| | Expression | ECO-R-NT | ECO-S-T | ECO-R-T | ECO-R-T |
| Total | Decrease | 348 | 1765 | 5639 | 846 |
| Genes | Increase | 2127 | 1774 | 153 | 281 |
| | Total | 2475 | 3539 | 5792 | 1127 |

Abiotic and biotic stress-responsive genes. Stress-induced genes are key not only in mediating the stressor but also lead to downstream signaling of the stressed state required for defense and tolerance to the stressor. Following treatment, ECO-S-T exhibited the repression of heat stress transcription factors and proteins. Several MYB (myeloblastosis) and MYC proteins were differentially expressed. MYB-related genes, specifically MYB44 (>3-fold change), act as transcription factor. Overexpression of MYB44 results in stomatal closure in the absence of ABA under drought, low temperature, or salinity stress [40]. MYC3 was induced 3.4-fold following treatment, indicating stimulation of jasmonic acid production. Jasmonic acid is another stress-signaling hormone. MYC3 can interact with MYB to regulate glucosinolate biosynthesis; compounds which are responsive to herbivory and form toxic compounds to insects [41]. Multiple disease response/resistance genes were repressed including-RPP13 like protein 4, RPM1, RGA4 and several putative resistance genes. Of note is the repression of the RGA4, which is one of a four-gene family residing at the same locus. RGA2 (>7.5-fold) and RGA3 (1.7) were induced following treatment. While all four members of the RGA family contain avirulence proteins, only RGA2 induces a resistant response to Pseudomonas infestans, restricting growth of the pathogen. To reduce the damaging build-up of hydrogen peroxide, several peroxidase genes-1, 2, 4, 15, 54, 52, 57, and multiple forms of these peroxidase transcripts were induced 2.8- to 7-fold. This is an expected response given the mode of action previously described, but these specific genes have not been described in response to propanil. Ethylene induction is a major component of stress signaling. However, the precursor enzymes for ethylene production [1-aminocycloproane-1-carboxylate (ACC) synthase (−2.7) and multiple ACC oxidase genes (−1.8 to −2.2)] were repressed 24 h after propanil treatment. This means that at this time, ethylene was not produced. It is possible that ethylene induction by herbicide stress occurred earlier, as indicated by the presence of multiple ethylene responsive transcription factors (ERF).
Induction of potential herbicide detoxifying enzymes. Xenobiotic detoxification genes were also investigated. Fifty-two transcripts were repressed while 30 were induced 24 h following treatment. Among the induced genes were members of five large gene families including ABC transporters, acetyltransferases, cytochrome P450s, glutathione-S-transferases, and glycosyltransferases (Table 5). Of these, the largest families are the cytochrome P450 (CYP), glutathione-S-transferase (GST), and glucosyltransferase (GT) proteins, all of which have been previously characterized in response to herbicide action. Four of the CYP genes are from the CYP71 family, which have not been characterized except for being similar to other members in this family. CYP71A1 is a component of the flavonoid biosynthetic process, induced by ethylene. CYP94C1 is associated with the jasmonic acid-mediated signaling pathway. The four propanil-induced GST genes have roles in endogenous and exogenous chemical glutathionylation, including herbicides, based on their similarity to previously described genes in the Uniprot database. GSTU1 and GSTU6 are members of Tau family of GST's which have known roles in xenobiotic detoxification [42]. UGT73B4 (3.8 & 5.7) have quercetin 3- and 7-O-glucosyltranferase activities but are also able to detoxify 2,4,6-trinitrotoluene (TNT), the explosive compound in dynamite [43]. UGT74D1 glycosylates jasmonate derivatives as well as IAA, and several components of the flavonoid biosynthetic process.

TABLE 5
Summary of the xenobiotic detoxification genes and
gene families with the corresponding fold change
induced within ECO-S following propanil treatment.

| Gene | | Fold |
|---|---|---|
| Family | Transcript ID | Change |
| ABC | ABC transporter G family member 42 | 1.5 |
| Transporter | ABC transporter G family member 5 | 2.8 |
| | ABC transporter G family member 53 | 2.3 |
| | ABC transporter G family member 53 | 4.7 |
| Acetyl- | Heparan-alpha-glucosaminide N-acetyltransferase | 2.5 |
| transferase | Uncharacterized acetyltransferase At3g50280 | 2.5 |
| | Uncharacterized acetyltransferase At3g50280 | 3.5 |
| Cytochrome | CYP716B1 | 3.0 |
| P450 | CYP71A1 | 2.9 |
| | CYP71A21 | 5.1 |
| | CYP71D7 | 5.9 |
| | CYP94C1 | 3.7 |
| | CYP94C1 | 5.4 |
| | CYP94C1 | 5.5 |
| Glutathione- | Probable glutathione S-transferase | 1.8 |
| S-transferase | GSTU1 | 2.5 |
| | GSTU1 | 2.7 |
| | GSTU6 | 2.3 |
| | GSTU6 | 4.1 |
| | MSR-1 | 2.1 |
| Glycosyl- | UGT73B4 | 3.8 |
| transferase | UGT73B4 | 5.7 |
| | UGT73C1 | 3.1 |
| | UGT73C2 | 3.3 |
| | UGT74D1 | 2.2 |
| | UGT75C1 | 2.9 |
| | UGT83A1 | 2.0 |
| | UGT83A1 | 3.6 |
| | UGT83A1 | 6.0 |

Concerted Repression of Gene Expression and Biological Networks in the Response of Herbicide-Resistant E. colona
Plant growth and maintenance gene response. Herbicide action resulted in a significant repression in plant activities. Only three ontological terms were enriched, one biological process term-flavonoid glucoridination, and two molecular function terms-quercetin 7 and 3-O-glucosyltransferase activity. In terms of gene expression, a total of 5,639 genes were repressed and only 153 induced genes (Table 4). Photosynthetic complex proteins were all repressed 24 h following treatment. This includes reduction of cytochrome c1, ferredoxin thioredoxin reductase, ATP synthase subunits (<−10), ferredoxin 6 (<−2.4), NADH-cytochrome b5 reductase proteins (<−7.4), and ubiquinol cytochrome-c reductase complex core protein (<−8.6). This followed a similar pattern of gene repression for carbon metabolism and nitrogen metabolism. Carbon assimilation genes including malate dehydrogenase (−10.8), pyruvate phosphate dikinase (−12), NADP-dependent malic enzyme (−9.3), pyruvate dehydrogenase E1 subunit (<−10.4), ADP sugar pyrophosphatase (−7.2), fructose-1,6-bisphosphatase (−9.3), and several other compounds modifying proteins were repressed. Glutamine synthetase was also repressed (−10.5-fold) following treatment. Similar to ECO-S-T, most genes associated with DNA and subsequent translation and transcription were repressed including many polymerase and topoisomerase proteins. The remaining TF transcripts have general functions in cis-acting DNA activation. In terms of sugar metabolism, again no transcripts were induced. However, TPS6, which was induced following treatment in ECO-S was repressed 6.1-fold. Two other trehalose transcripts, both for trehalose phosphorylase were repressed. These have a role in the catabolism of trehalose [44]. Two sugar transporters, ERD6-like 8 and SWEET2a, were also repressed (4.2- and 3.8-fold, respectively). ACCase (−9.7) and ACCase 2 (−10.2), as well as acetyl-coenzyme A synthetase (−8.4), and phenolic glucoside malonyltransferase 1 (−3.5) were all repressed. Based on the broad-spectrum repression of key metabolic genes across all major biochemical pathways, ECO-R seemed to be at a quiescent physiological state 24 h after treatment with propanil.
Stress-responsive gene expression. In terms of stress response, the results were similar to the plant growth genes previously described. A general repression in stress-induced genes occurred following propanil application, with only two transcripts induced. Similar to MYC3 induced in ECO-S-T, MYC2 is a transcriptional activator involved in jasmonic acid regulation and can complex with MYB proteins to regulate glucosinolate biosynthesis. It has a secondary role of regulating ABA response under drought conditions, inducing rd22 a gene responsible for alleviating drought stress and induced by ABA [45]. Several heat shock proteins were repressed by as much as −9.9-fold. While no peroxidase genes were induced, one was repressed-peroxidase 5 (−3.1). ACC oxidase is also repressed (−4.5) and given ethylene's role in stress response signaling, this indicates a contrast to ECO-S-T. However, three ERFs were induced-ERF060 (2.1 & 2.1) and ERF7 (6.7), providing some indication of ethylene biosynthesis early in the response process. Both proteins interact with the GCC-pathogenesis promoter sequence but ERF060 is an activator while ERF7 is a repressor. Stress enhanced protein 2 (2.5) and disease resistance protein RPM1 (7.1) were two of the induced stress-response genes. RPM1, induces a hypersensitive response following recognition of P. syringae avirulence proteins. Stress enhanced protein 2 (SEP2), is a unique protein believed to act as a photo-oxidative protectant, against ROS and cellular degradation [46].
Xenobiotic detoxification genes in response to propanil. Other stress-induced genes, which are potentially involved in xenobiotic detoxification for ECO-R-T are listed in Table 6. Only one gene, UGT73C2, was induced in both ECO-R-T and ECO-S-T; the remaining transcripts were different. CYP709B2 and CYP72A15 are of interest because of the multiple transcript variants, and high level of induction following treatment compared to the other cytochrome P450 genes. GSTU17 is also of note given its role in light signaling and morphogenesis which utilizes phytohormone signals to direct developmental changes [47]. The remaining detoxifying genes require further research to elucidate the impact they have on herbicide resistance.

TABLE 6
Summary of the xenobiotic detoxification genes and gene
families with the corresponding fold change induced
within ECO-R following propanil treatment.

| | | Fold |
|---|---|---|
| Gene Family | Transcript ID | Change |
| ABC Transporter | ABC transporter C family member 10 | 7.4 |
| Cytochrome | CYP72A11 | 3.1 |
| P450 | CYP89A2 | 2.5 |
| | CYP89A2 | 3.8 |
| | CYP89A2 | 3.6 |
| | CYP709B2 | 8.6 |
| | CYP709B2 | 3.9 |
| | CYP72A13 | 3.8 |
| | CYP72A15 | 2.6 |
| | CYP72A15 | 3.4 |
| | CYP72A15 | 2.9 |
| | CYP709B2 | 4.7 |
| | CYP709B1 | 7.0 |
| Glucosidase | Beta-glucosidase 22 | 3.6 |
| Glutathione-S- | GSTU17 | 7.9 |
| transferase | GST23 | 6.7 |
| Glycosyl- | UGT73E1 | 3.0 |
| transferase | UGT73C2 | 4.7 |
| | UGT83A1 | 2.3 |
| | UGT74G1 | 4.6 |
| | UGT74G1 | 4.5 |
| | UGT73D1 | 5.3 |
| | UGT73D1 | 6.9 |
| | UGT73D1 | 4.8 |
| | UGT75D1 | 7.4 |

### Comparative Overall Response of ECO-S and ECO-R to Propanil

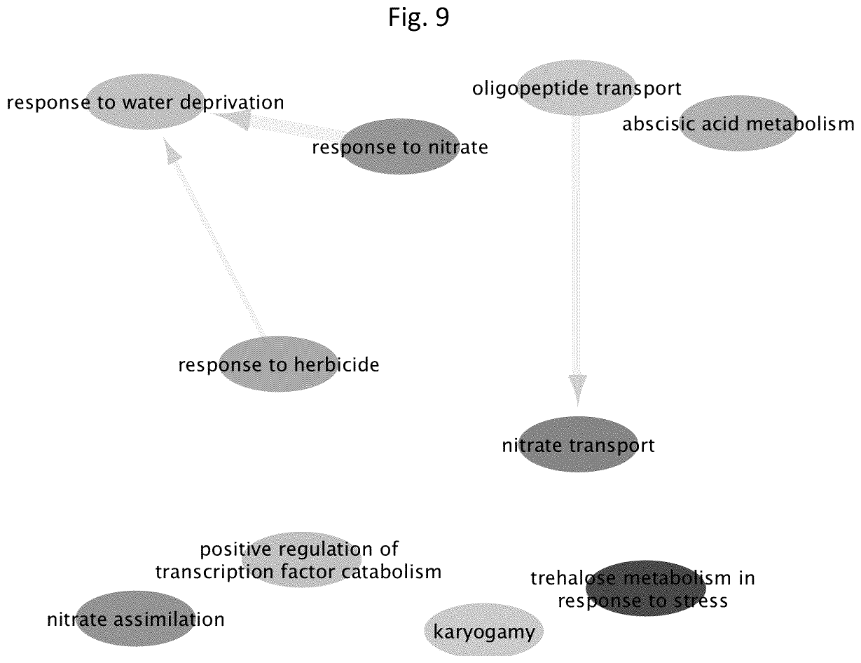

Gene ontology analysis revealed no significant enrichment of terms in ECO-R-T, but several were enriched in ECO-S-T (FIG. 9). A supercluster identified as ‘trehalose metabolism in response to stress’ was formed, which was composed of overexpressed terms related to response to nitrate, response to herbicide, nitrate assimilation, ABA metabolism, and response to water deprivation. Of the multiple enriched terms, the trehalose-metabolism-in-response-to-stress term, was the most over-represented, as observed in the induction of genes in ECO-S-T described previously.

For comparatively repressed genes in ECO-R-T, 846 transcripts were repressed in ECO-R-T compared to ECO-S-T (Table 4). In terms of biological functions necessary for growth activities including photosynthesis, carbohydrate metabolism, sugar metabolism, and nitrogen metabolism, the majority of the genes were at a lesser abundance in ECO-R-T. These include transcripts for ferredoxin-6 (−6.5), nitrate reductase (<−6), nitrate transporters (<−4), and several trehalose biosynthesis enzymes (<−3). In terms of ABA action, several transcripts for ABA 8′-hydroxylase 1 (<−6.5) and two ABA receptors PYL5 (−4.8) and PYL8 (−3.7) were significantly repressed in ECO-R-T. Several ethylene-responsive transcription factors were comparatively repressed; however, only one ACC oxidase homolog was repressed in ECO-R-T. Finally, in terms of herbicide detoxification several GT and GST enzymes were repressed, indicating their reduced if not ineffective role in herbicide resistance. One cytochrome P450-CYP704C1, was significantly repressed (−7.9).

A total of 281 transcripts were elevated in ECO-R-T more so than ECO-S-T. Several transcription factors, mostly from the WRKY family, which are elicitor-responsive proteins that interact with the W box segment of DNA, were induced. DNA-directed RNA polymerase 1 subunit RPA112 III (10.3) and polymerase III subunit 2 (10.8) were also enhanced. The latter, functions in the synthesis of small RNA's, regulatory RNA fragments, which may be beneficial for coordinating the abiotic stress response. Phosphoenolpyruvate carboxylase (PEPc) kinase 1 was induced (>5). This is a protein essential for the activation of (PEPc) for the production of oxaloacetate, a primary component of plant metabolism. Activation of the ethylene biosynthetic pathway was also observed. Several ACC oxidase transcripts (3.3 to 4.1-fold) and an ACC synthase transcript (8.6) were present at a much higher level in ECO-R-T. In turn, three ERF-4 (4.3), 7 (8), and 11 (3.4), were induced; all are transcriptional repressors of the GCC-box pathogenesis promoter. Xenobiotic detoxifying enzymes were present in significantly higher concentrations in ECO-R-T. Transcripts for CYP704C1 (7.7), CYP71A1 (3.6), CYP72A15 (6.6 & 11.3), and CYP709B2 (3 & 10.4) were significantly induced following herbicide treatment. Also, GSTU17 (10.9), GST1 (3.9), and GST4 (>5.3) were all at a greater abundance. Three GT enzymes were induced including UGT88A1 (3.2), UGT88F3 (5.9), and UGT73D1 (7.3). Beta-glucosidase 22 (5.9) was also present.

### DISCUSSION

E. colona Response to Propanil Involves the Abiotic Stress Response Pathway Driven by ABA Flux

ABA is a critical phytohormone necessary for the activation and downstream signaling of multiple abiotic stress responses, particularly in response to water deprivation [48]. Given its role in various activities, the signaling pathway and its implications have not been described in terms of signaling herbicide-induced response. While ABA itself functions to reduce stomatal conductance, it also directs several activities via calcium-dependent channels in the plant that lead to responses to abiotic stress [49]. High ABA concentrations alone can limit photosynthesis which in itself is detrimental to carbon assimilation and leads to cessation of grown, cellular disruption and even potentially plant death [50]. The response of ECO-S-T and ECO-R-T to propanil highlights the involvement of ABA in plant response herbicide (at least with propanil). The ECO-S-T transcriptome was greatly enriched with terms indicative of an ABA-mediated response, including ABA metabolism, response to nitrate, and response to water deprivation. Several genes were induced following treatment including two ABA receptors-PYL5 and PYL8 enzymes, signaling the potential presence of elevated ABA concentrations in the plant that might lead to stomatal closure. Induction of PYL5 and PYL8 have been shown to enhance resistance to drought via stomatal closure [51]. This is indicative of the plant attempting to slow photosynthesis in the presence of a photosynthesis inhibitor, such as propanil. MYB44, as well as other MYB-like proteins, were also expressed in ECO-S-T, which in the absence of ABA would reinforce stomatal closure, serving as a secondary factor that limits photosynthetic activity [45,40]. In terms of calcium transport directed by ABA, several calcium exchanger proteins, calcium-dependent protein kinases, and a calcium binding protein CLM36 were induced. This further implicates ABA-directed activities following treatment. The presence of ABA hydroxylase genes at the magnitude of induction observed in ECO-S-T is indicative of the high levels of ABA present 24 HAT. These proteins were not induced in ECO-R-T. Instead, the ABA hydroxylase and PYL5 and PYL8 genes were constitutively enhanced in ECO-R compared to ECO-S (data not shown). Treatment with propanil did not illicit further increase in expression of these genes, indicating that the native levels were sufficient to signal herbicide effects and initiate mitigation processes.

E. colona Response to Propanil is Also Tightly Linked with Biotic Stress Responses
Jasmonic acid-mediated response. Biotic response characterization is as important as characterization of the abiotic stress signaling and response pathway. The jasmonic acid pathway as well as the general plant defense response were activated by propanil in both ECO-S-T and ECO-R-T. This was indicated by the induction of transcription factors MYC3 and MYC2 in ECO-S-T and ECO-R-T, respectively. MYC2 is directed by the action of ABA, complexing with MYB to impart ABA directed drought tolerance [45]. This interaction is also capable of producing glucosinolates, which are compounds toxic to insects and deter herbivory [41]. While these glucosinolates and the jasmonic acid activity may not be involved in herbicide resistance, the signal transduction allows us to connect the abiotic stress response to biotic stress response. Specifically, the the ABA activities are apparently inducing a wider whole-plant response that overlaps the jasmonic acid pathway, mediated through the MYC proteins [52]. While the induction of ABA related genes is much lesser in ECO-R-T, the high basal production of ABA would have stimulatory effects on the jasmonic acid pathway following treatment given these connections.
Disease resistance response and the implications for resistance. Disease resistance transcripts were also rampant across both the ECO-S-T and ECO-R-T responses; interestingly they often did not have similar transcript expression patterns. Important to general disease or pathogen infection is the induced hypersensitivity response to the avirulence proteins of certain pathogens. The hypersensitive response is characterized by an intermittent burst of hydrogen peroxide that results in cell death at the site of infection, limiting the movement of the pathogen out of the infected area [53]. The four-member RGA family of disease resistance proteins were differentially expressed across ECO-S-T. RGA4 transcripts were repressed in ECO-S-T while RGA2 and RGA1 were induced following treatment. While these genes act in concert, because of their similar positions on the locus, RGA2 is the only protein, which recognizes and responds to the avirulence protein. Transcripts of disease resistance protein RPM1 were more prevalent in ECO-R-T but also observed in ECO-S-T. RPM1 is another avirulence recognition protein that incites an oxidative response that leads to a hypersensitive reaction [54]. These among several other non-specific disease resistance proteins were all expressed. These proteins may contribute to herbicide response, and eventual resistance, by restricting the movement of the herbicide following treatment; such as observed in glyphosate-resistant Ambrosia trifida populations [55]. In this case, rapid cell death caused by ROS imparts a high level of resistance to glyphosate [56]. Within 24 hours of treatment, propanil action in R and S populations results in lesions on the leaf surface, often described as necrosis or leaf burn. In some instances including with ECO-R, resistant plants would appear completely necrotic and dead within one week of treatment, but regenerate to a healthy plant by three weeks, as described by so-called ‘phoenix-resistance’ [personal observation, 56]. This response may be imparted by these hypersensitive pathogen-response genes. The difference between in response between S and R may be based on the presence or absence of these avirulence protein genes and their action. More importantly, the hypersensitive type response may be imparted as a abiotic stress avoidance mechanism. Multiple peroxidase genes were greatly induced in ECO-S-T, while none were induced in ECO-R-T. While the action of the peroxidase genes helps to alleviate the oxidative stress, this may allow the herbicide to move and become more destructive, resulting in prolonged exposure to propanil, leading to death. The ‘hypersensitive-response’ in ECO-R-T may contribute to the resistance mechanism of the plant. This would require biochemical validation and physiological assessment.

### Trehalose Biosynthesis has a Role in ECO-S Response to Herbicide and ECO-R Predisposition to Tolerate the Herbicide

Trehalose is a non-reducing sugar that has been implicated in abiotic stress tolerance in several plant and bacterial species [58,59]. This sugar can impart several properties to the plant including tolerance to dehydration, enhancement of photosynthesis, and scavenging ROS [60,61]. Its intermediate, trehalose-6-phosphate (Tre6P), is also a major constituent in sucrose signaling and starch to sucrose conversion in the plant, capable of coordinating many growth processes. This would result In a larger plants [62]. Trehalose induction was noted in ECO-S-T in both the network assessment and the gene expression profile whereby both of the precursory enzymes TPS and TPP were induced, but a significant induction was not observed in ECO-R-T. This provides evidence that no significant change in the regulation has occurred. Unlike ECO-S—N, ECO-R both constitutively upregulates both TPS and TPP without and following herbicide treatment, as implicated by both the gene ontology clusters and the differential gene expression profiles. This means that the trehalose is present at the time of application and the onset of the abiotic stress response. The induction of trehalose biosynthesis in ECO-S-T is of note as this is the first observation of trehalose biosynthesis involvement in herbicide response. Trehalose may provide several benefits to the plant following herbicide application; however, the concomitant decrease in photosynthesis and carbon assimilation processes may mean this response is transient. The lack of trehalose supply would limit its activity.

In contrast, ECO-R-N has constitutive enhancement of the genes necessary to produce trehalose as well as elevated carbon assimilation activities compared to ECO-S-N (data not presented). Free trehalose and Tre6P may have active roles in the potential for the plant to survive treatment with a photosynthesis inhibitor when paired with upregulation of a detoxifying enzyme. Trehalose may be acting as an integral membrane stabilizer to protect not only against oxidative damage caused by ROS but also those produced in the hypersensitive response, described previously. The sugar moiety is able to stabilize protein membranes by connecting itself with the polar heads of the lipid bilayer, forming hydrogen bonds, subsequently stabilizing the membrane by preventing phase transition and leakage [59,60,63]. The trehalose may also serve as an osmolyte that allows for the hypersensitive response to occur in the leaf tissues and the plant to regenerate from the meristematic zone of the grass [61]. This would make it a somewhat unique feature to grasses, as the meristematic regions are at or below the soil surface, making them less affected by the photo-oxidative damage occurring in the leaves. The build-up and storage of trehalose in the plant may be the necessary source for prolonged growth following severe photo-oxidative damage. Finally, another key component of survival for the ECO-R population may be the presence of transcripts for trehalose phosphorylase. This is a key enzyme involved in the catabolism of trehalose into glucose 6-phosphate and beta-D-glucose 1-phosphate, two compounds with active roles in several biological processes [44]. Under severe stress the plant would require both compounds, glucose 6-phopshate in particular, to not only regenerate by serving as an energy source, but as a substrate in several metabolic processes. This not only provides a basic framework for the role of trehalose in protection against herbicides but implicates it as a pivotal compound in the prolonged activities of plants following herbicide treatment.

### Herbicide Resistance in ECO-R is Driven by Coordinated Induction of Cytochrome P450 Hydroxylation and Glutathione Conjugation

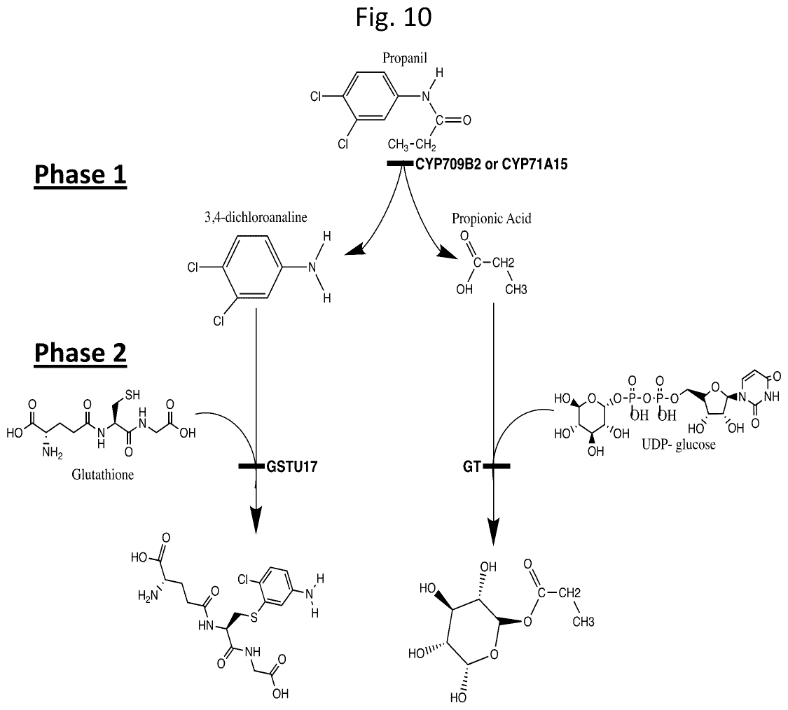

Initial investigation into the herbicide resistance mechanism included a search or the aforementioned aryl-acylamidase protein transcript but it was not present within the transcriptome response profile. Based on the transcriptome profiles of ECO-R-T before and after propanil treatment and the comparative analysis between ECO-R-T and ECO-S-T, it is possible that we have identified both the primary and secondary mechanisms of propanil detoxification (FIG. 10). First, the oxidative step may involve either CYP709B2 and/or CYP72A15. Both have near identical expression profiles and would provide the necessary hydroxylation to reduce propanil into the two products-3,4 dichloroaniline and propionic acid; as observed with the aryl-acylamidase protein [31]. The shear abundance of their transcripts and expression profiles across differential expression analysis implicate the role of these enzymes in propanil detoxification. CYP72A15 has not been described in the literature while some research on CYP709B2 has been conducted. A sister gene in the family-CYP709B3, has been investigated for its role in ABA- and salt stress response; increasing ABA levels do result in the induction of CYP709B2 but serve no function in alleviating the stressor [64]. Members of the CYP709 subfamily are stimulated in response to IAA which is also induced under stress [65]. The secondary step in the detoxification pathway is the conjugation of metabolites to an endogenous moiety usually sugar (GT) or glutathione (GST) [23]. While several GT enzymes were induced, they serve various roles in the plant that are less associated with the detoxification process. However, given their affinity for carboxylic acid side chains, one of the GT enzymes may act on the propionic acid derivative [66]. Several of the GST enzymes are members of the tau family of GST's, with known roles in exogenous xenobiotic detoxification including herbicides [42]. One in particular GSTU17, has been investigated due to its response to light and plant hormones including ABA [47]. This study also investigated its affinity to the substrate 1-chloro-2,4-dinitrobenzene, a compound similar in structure to propanil and the 3,4-dichloroanaline metabolite, containing both a chloride and nitro group. The chloride side group serves as an indicator of potential GST activity whereby a nucleophilic substitution can occur and the thiol group of glutathione can form a bond to the benzene ring [67]. Given the gene expression profile and known substrates for the enzyme, this is most likely the secondary step in the xenobiotic detoxification process for propanil in ECO-R. This requires further validation in bacterial and plant systems to verify the hypothesized interactions.

### CONCLUSIONS

Herbicide resistance is a complex polygenic response to the imposed abiotic stress from herbicide action. Continuous selection pressure imposed not just by herbicides but general management, selects for a variety of traits that can be classified as both domesticated but also weedy. The weed management process allows for the adaptive evolution in the face of adversity, and the traits evolved for specific tolerances or resistance cannot simply be identified in isolation and considered as monogenic. This is best exemplified by the propanil response and herbicide resistant transcriptome outline here. By contrasting the low-tolerance phenotype of ECO-S to that of the multiple and highly propanil-resistant ECO-R, with and without treatment, we conclude that three pathways were responsive to propanil: abscisic acid, jasmonic acid, and the trehalose biosynthetic pathways. While the responses are not identical, key features such as the altered regulation of the phyothormones, the impact of hypersensitive responses, and the use of trehalose to mediate the negative effects or secondary or tertiary herbicide activity are present. Using the transcriptome and the comparative analysis between gene ontology and expression profiles, we are able to provide a novel herbicide resistance pathway that may be employed by ECO-R. Two cytochrome P450 genes CYP709B2 and CYP72A15, with the ability to detoxify propanil into its hydroxylated substrates, clearly take action in response to the herbicide as indicated by their respective expression profiles. Given the presence of multiple GT and GST enzymes transcripts the potential for secondary interaction with the products 3,4 dichloroanaline and propionic acid is also highlighted. Collectively, this research provides the first holistic understanding and documentation of stress-responsive genes affected by propanil in a multiple-resistant E. colona population. This work not only demonstrates the utility of transcriptomics in understanding weed biology and physiology, but provides gene expression and literature support for the findings.

### Methods

### Plant Materials

Accession source and population profiling. From 2010 to 2016, the University of Arkansas weed physiology group has been collecting putative herbicide-resistant Echinochloa populations from rice production fields throughout the state of Arkansas. These accessions survived late into the season following early herbicide applications for management. Collections took place in the late summer to early fall prior to rice harvest; these accessions were bulk sampled by in-field location and farm, with field histories collected when possible. All of the samples were submitted to the University of Arkansas Altheimer Laboratory for assessment of their resistance profile and characterized according to their species. Results for the herbicide resistance screening and species abundance, including, the methodology used for resistance profiling are presented in Rouse et al. [10]. Populations with unique profiles and of interest for further research were then grown in isolation to produce pureline seed. Due to the low outcrossing rate of E. colona, a single generation has been determined as adequate for production of homozygous individuals. E. colona resistant (ECO-R) was collected in 2010 from Lawrence County, Arkansas, and selected for this research due to its unique multiple-resistant profile to three rice herbicides-cyhalofop (˜2× field dose), propanil (>8× field dose), and quinclorac (>32× dose), and one soybean herbicide-glufosinate (˜2× field dose) (data not presented). Another accession, E. colona susceptible (ECO-S), was also selected and grown in isolation. This accession was selected because of its similar cropping history and geographic location, as well as its high level of susceptibility to the herbicides of interest. Propanil tolerance is observed at approximately a 3× field dose, which is common to the state of Arkansas but a sufficient contrast to the ECO-R phenotype.
Plant Treatment and Processing for RNA-sequencing. Pure-line generated seed of both the ECO-R and ECO-S accessions were germinated in square pots with commercial potting soil in a growth chamber set to 14-hr day length, 33 C day temperature, and 24 C night temperature. Each pot was replicated twice to provide two biological replications of each herbicide by accession combination. Within each pot, a single plant was maintained prior to herbicide application for approximately two weeks. When the plants reached the two fully expanded leaf growth state, they were treated with propanil (4.5 kg ha-1+nonionic surfactant at 0.25% v/v). The plants designated to receive the treatment were treated simultaneously in a motorized spray chamber calibrated to deliver 187 L ha-1 from a 250-mL tank volume. After the plants were allowed to dry, approximately 30 mins, both the treated and nontreated counterpart were labeled as either ECO-S/R-N (for nontreated) or ECO-S/R-T (for the treated), and moved back into the growth chamber. Exactly 24 hours after application, the above ground portion of the plants, including both the shoot and leaf tissues, were harvested and immediately submerged in liquid nitrogen. The samples were then transferred to RNAlater™-ICE (Invitrogen, Carlsbad, CA, USA) for shipment to the Clemson University Genomics Institute, in Clemson (CUGI), South Carolina.

### RNA Extraction, Transcriptome Assembly, and Annotation

The processes and methodology for the RNA extraction, RNA-sequencing, transcriptome assembly, and transcriptome annotation is outline in Rouse et al. [37]. A brief summary is included here to provide a cohesive understanding of the research and analysis pipeline. Total RNA was extracted according to the manufacturer's instructions for the commercially available kit. The paired-end library was prepared using the TruSeq Stranded Total RNA kit (Illumina Inc., SandDiego, CA, USA). Cytoplasmic and mitochondrial rRNA was removed to improve the quality of results. Following RNA fragmentation, the RNA was reverse-transcribed into cDNA using random primers and then second strand of cDNA was synthetized based on the cDNA template. These fragments were then tagged with an additional ‘A’ and the adapter for sequencing. The final cDNA library was prepared from PCR enriched and tagged sequences. All of the samples were submitted to the Holdings Cancer Center at the Medical University of South Carolina, Charleston, SC, USA where they were sequenced on an Illumina Hiseq 2500 platform. The de novo transcriptome was assembled from the sequenced results. The data files were assembled using the Trinity RNA-Seq pipeline (Babraham Institute, Cambridge, UK). Following data quality checks and processing to normalize the samples using the TrinityRNASeq 2.2.0 software, the transcriptome was assembled using Trinity and Transdecoder 3.0.1 (Broad Institute. Following assembly, the Trinotate 2.0 software package was used for the functional annotation of the transcriptome by homology to BLAST+ and Swissprot data bases. Both the HMER and PFAM were used to generate the necessary information for the primary annotation databases which included eggNOG, GO, and KEGG.

### Differential Gene Expression Analysis

The aforementioned transcriptome was used in all subsequent analysis for a description of the treatments of interest. A GTF file was generated for comparing each of the samples, T and N, to the reference transcriptome. Feature counts were generated using the Subread package (subread), which were used to quantify the differentially expressed transcripts for both replications that were paired concordantly, ensuring proper analysis given the differences between the two replications. Using the R statistical software (.r-project), the edgeR package developed by Bioconductor_(bioconductor), quantified the filtered raw counts from the RNA sequencing with standard normalization performed using the trimmed mean of M-values applied to the counts [68,69]. Each set of counts were fit using a GLM model for determination of significance (p≤0.01). For each comparison of interest, including the nontreated and treated conditions, a likelihood ratio test (LRT) was performed to identify the fold differences. The analysis was then further evaluated using a false discovery rate for p-value correction to reduce the error in the results.

The raw output from these results were used to generate a table of annotated data for the comparisons of interest as well as the statistical values and log 2-fold changes for each of the transcripts of interest. This information was further qualified manually to categorize important genes into functionally relevant categories including carbon assimilation, photosynthesis, sugar synthesis, fatty acid synthesis, stress signaling, ethylene biosynthesis, nitrogen metabolism, and herbicide detoxification. Several subcategories were assembled for each. For herbicide detoxification, a review of literature revealed several key categories that were investigated in the analysis including ABC transporters, aminotransferases, cytochrome P450s, glutathione-S-transferase, glucosyltransferases, and glucosidases [23,70,71]. To describe each of the genes and pathways for the associated genes, both the Uniprot and KEGG [73] databases were used for basic descriptions. To process the large quantities of data for characterization and to identify various overlapping gene profiles, JMP Pro 13 (SAS institute, Cary, NC) with the Venn diagram add on package was used.

### Gene Ontology Analysis

The Trinotate output was used with the ‘goseq’ package from Bioconductor to assign GO terms to the transcripts from the transcriptome. The enrichment analysis was performed on the transcripts, which have been expressed at a log 2 fold change of <−2 or >2 and a p-value of ≤0.01. This generated an over represented p-value which was used to assess the significance of each term. The results of the analysis were visually assessed using REVIGO (revigo) to generate superclusters that share overlapping terminologies based on semantic similarity. The output from this clustering was then visualized with the Cytoscape Network Analysis software (cytoscape). For description of the gene ontology terms and functions of the terms EggNOG and the GO Consortium databases were used.

### REFERENCES FOR EXAMPLE 2

1. Chauhan B S, Jabran K, Mahajan G. Rice Production Worldwide. Cham, Switzerland: Springer; 2017.
2. Valverde B E, Riches C R, Caseley J C. Prevention and management of herbicide resistant weeds in rice: Experiences from Central America with Echinochloa colona. 2000.
3. Van Wychen L. 2015 Baseline Survey of Most Common and Troublesome Weeds in the United States and Canada. In: Weed Science Society of American National Weed Survey Dataset [Internet]. 2015 [cited 22 Mar. 2017].
4. Danquah E Y, Johnson D E, Riches C, Arnold G M, Karp A. Genetic diversity in Echinochloa spp. collected from different geographic origins and within rice fields in Cote d′Ivoire. Weed Res. 2002; 42:394-405. doi: 10.1046/j.1365-3180.2002.00300.x
5. Barrett S H. Crop mimicry in weeds. Econ Bot. 1983; 37:255-282. doi: 10.1007/BF02858881
6. Yang X, Fuller D Q, Huan X, Perry L, Li Q, Li Z, et al. Barnyard grasses were processed with rice around 10000 years ago. Sci Rep. Nature Publishing Group; 2015; 5:16251. doi: 10.1038/srep16251
7. Smith R J. Weed Competition in Rice. Weed Sci. 1968; 16:252-255.
8. Smith R J. Competition of barnyardgrass with rice cultivars. 1974; 22:423-426.
9. Smith R J. Weed Thresholds in Southern U.S Rice, Oryza sativa. Weed Technol. 1988; 2:232-241.
10. Rouse C E, Burgos N R, Norsworthy J K, Tseng™, Starkey C. Echinochloa resistance to herbicides continues to increase in Arkansas rice fields. Weed Technol. 2017; In Press.
11. Bryson C, Reddy K N. Diversity of Echinochloa in the mid south. Proceedings of the 2012 Weed Science Society Annual Meeting. Honolulu, HI: Weed Science Society of America; 2012.
12. Burgos N R, Rouse C E, Tseng™, Abugho S B, Hussain T, Salas R A, et al. Resistance Profiles of Echinochloa colona in Arkansas. 68th Southern Weed Science Society Annual Meeting. Savannah, GA: Southern Weed Science Society; 2015. p. 22.
13. Talbert R E, Burgos N R. History and Management of Herbicide-resistant Barnyardgrass (Echinochloa Crus-galli) in Arkansas Rice. Weed Technol. 2007; 21:324-331. doi: 10.1614/WT-06-084.1
14. Cobb A H, Reade J P. Herbicides that inhibit photosynthesis. Herbicides and Plant Physiology. 2010. pp. 89-115.
15. Frear D S, Still G G. The metabolism of 3, 4-dichloropropionanilide in plants. Partial purification and properties of an aryl acylamidase from rice. Phytochemistry. 1968; 7:913-920. doi: 10.1016/S0031-9422 (00) 82175-5
16. Hoagland R E, Norsworthy J K, Carey F, Talbert R E. Metabolically based resistance to the herbicide propanil in Echinochloa species. Weed Sci. 2004; 52:475-486. doi: 10.1614/ws-03-039r
17. Heap I. International Survey of Herbicide Resistant Weeds [Internet]. 22 Aug. 2017 pp. 145-153.
18. Norsworthy J K, Bond J, Scott R C. Weed management practices and needs in Arkansas and Mississippi rice. Weed Technol. 2013; 27:623-630. doi: 10.1614/WT-D-12-00172.1
19. Norsworthy J K, Ward S M, Shaw D R, Llewellyn R S, Nichols R L, Webster™, et al. Reducing the Risks of Herbicide Resistance: Best Management Practices and Recommendations. Weed Sci. 2012; 60:31-62. doi: 10.1614/WS-D-11-00155.1
20. Devine M D, Shukla A. Altered target sites as a mechanism of herbicide resistance. Crop Prot. 2000; 19:881-889. doi: 10.1016/S0261-2194 (00) 00123-X
21. Gardner S N, Gressel J, Mangel M. A revolving dose strategy to delay the evolution of both quantitative vs major monogene resistances to pesticides and drugs. Int J Pest Manag. 1998; 44:161-180. doi: 10.1080/096708798228275
22. Yuan J S, Tranel P J, Stewart C N. Non-target-site herbicide resistance: a family business. Trends Plant Sci. 2007; 12:6-13. doi: 10.1016/j.tplants.2006.11.001
23. Kreuz K, Tommasini R, Martinoia E. Old Enzymes for a New Job. Plant Physiol. 1996; 349-353.
24. Riar D S, Norsworthy J K, Srivastava V, Nandula V, Bond J A, Scott R C. Physiological and molecular basis of acetolactate synthase-inhibiting herbicide resistance in barnyardgrass (Echinochloa crus-galli). J Agric Food Chem. 2013; 61:278-289. doi: 10.1021/jf304675j
25. Lopez-Martinez N, Marshall G, De Prado R. Resistance of barnyardgrass (Echinochloa crus-galli) to atrazine and quinclorac. Pestic Sci. 1997; 51:171-175. doi: 10.1002/(SICI) 1096-9063 (199710) 51: 2<171::AID-PS612>3.0.CO; 2-7
26. Alarcón-Reverte R, García A, Urzúa J, Fischer A J. Resistance to Glyphosate in Junglerice (Echinochloa colona) from California. Weed Sci. 2013; 61:48-54. doi: 10.1614/WS-D-12-00073.1
27. Alarcón-Reverte R, García A, Watson S B, Abdallah I, Sabaté S, Hernández M J, et al. Concerted action of target-site mutations and high EPSPS activity in glyphosate-resistant junglerice (Echinochloa colona) from California. Pest Manag Sci. 2015; 71:996-1007. doi: 10.1002/ps.3878
28. Yasuor H, Zou W, Tolstikov V V, Tjeerdema R S, Fischer A J. Differential oxidative metabolism and 5-ketoclomazone accumulation are involved in Echinochloa phyllopogon resistance to clomazone. Plant Physiol. 2010; 153:319-326. doi: 10.1104/U.S. Plant Pat. No. 110.153296
29. Hamza A, Derbalah A, El-Nady M. Identification and mechanism of Echinochloa crus-galli resistance to fenoxaprop-p-ethyl with respect to physiological and anatomical differences. ScientificWorldJournal. 2012; 2012:893204. doi: 10.1100/2012/893204
30. Yasuor H, Milan M, Eckert J W, Fischer A J. Quinclorac resistance: A concerted hormonal and enzymatic effort in Echinochloa phyllopogon. Pest Manag Sci. 2012; 68:108-115. doi: 10.1002/ps.2230
31. Hoagland R E, Graf G, Handel E D. Hydrolysis of 3,4-dichloropropionanilide by plant aryl acylamidases. Weed Res. 1974; 14:371-374. doi: 10.1111/j.1365-3180.1974.tb01077.x
32. Carey V F, Hoagland R E, Talbert R E. Resistance mechanism of propanil-resistant barnyardgrass: II. In-vivo metabolism of the propanil molecule. Pestic Sci. 1997; 49:333-338. doi: 10.1002/(SICI) 1096-9063 (199704) 49: 4<333::AID-PS541>3.0.CO; 2-0
33. Malik M, Burgos N, Talbert R. Confirmation and control of propanil-resistant and quinclorac-resistant barnyardgrass (Echinochloa crus-galli) in rice. Weed Technol. 2010; 24:226-233. doi: 10.1614/WT-09-053.1
34. Weller S C, Bressan R A, Goldsbrough P B, Fredenburg T B, Hasegawa P M. The effect of genomics on weed management in the 21st century. Weed Sci. 2001; 49:282-289. doi: 10.1614/0043-1745 (2001) 049 [0282: TEOGOW] 2.0.CO; 2
35. Stewart C N, Tranel P J, Horvath D P, Anderson J V., Rieseberg L H, Westwood J H, et al. Evolution of Weediness and Invasiveness: Charting the Course for Weed Genomics. Weed Sci. 2009; 57:451-462. doi: 10.1614/WS-09-011.1
36. Yang X, Yu X Y, Li Y F. De novo Assembly and Characterization of the Barnyardgrass (Echinochloa crus-galli) Transcriptome Using Next-Generation Pyrosequencing. PLOS One. 2013; 8. doi: 10.1371/journal.pone.0069168
37. Rouse C E, Saski C A, Noorai R E, Shankar V, Burgos N R. Multiple herbicide resistance involving quinclorac in Echinochloa colona involves adaptive co-evolution of abiotic stress- and xenobiotic detoxification genes. University of Arkansas. 2017.
38. Lee R M, Thimmapuram J, Thinglum K A, Gong G, Hernandez A G, Wright C L, et al. Sampling the Waterhemp (Amaranthus tuberculatus) Genome Using Pyrosequencing Technology. 57:463-469. 2009; 57:463-469. doi: 10.1614/WS-09-021.1
39. Gaines T A, Lorentz L, Figge A, Herrmann J, Maiwald F, Ott M C, et al. RNA-Seq transcriptome analysis to identify genes involved in metabolism-based diclofop resistance in Lolium rigidum. Plant J. 2014; 78:865-876. doi: 10.1111/tpj.12514
40. Jung C, Seo J S, Han S W, Koo Y J, Kim C H, Song S I, et al. Overexpression of AtMYB44 Enhances Stomatal Closure to Confer Abiotic Stress Tolerance in Transgenic Arabidopsis. Plant Physiol. 2007; 146:623-635. doi: 10.1104/U.S. Plant Pat. No. 107.110981
41. Schweizer F, Fernandez-Calvo P, Zander M, Diez-Diaz M, Fonseca S, Glauser G, et al. Arabidopsis Basic Helix-Loop-Helix Transcription Factors MYC2, MYC3, and MYC4 Regulate Glucosinolate Biosynthesis, Insect Performance, and Feeding Behavior. Plant Cell. 2013; 25:3117-3132. doi: 10.1105/tpc. 113.115139
42. Cummins I, Dixon D P, Freitag-Pohl S, Skipsey M, Edwards R. Multiple roles for plant glutathione transferases in xenobiotic detoxification. Drug Metab Rev. 2011; 43:266-280. doi: 10.3109/03602532.2011.552910
43. Gandia-Herrero F, Lorenz A, Larson T, Graham I A, Bowles D J, Rylott E L, et al. Detoxification of the explosive 2,4,6-trinitrotoluene in Arabidopsis: Discovery of bifunctional O- and C-glucosyltransferases. Plant J. 2008; 56:963-974. doi: 10.1111/j.1365-313 X.2008.03653.x
44. Andersson U, Levander F, Radstrom P. Trehalose-6-phosphate Phosphorylase Is Part of a Novel Metabolic Pathway for Trehalose Utilization in Lactococcus lactis. J Biol Chem. 2001; 276:42707-42713. doi: 10.1074/jbc.M108279200
45. Abe H, Yamaguchi-Shinozaki K, Urao T, Iwasaki T, Hosokawa D, Shinozaki K. Role of arabidopsis MYC and MYB homologs in drought- and abscisic acid-regulated gene expression. Plant Cell. 1997; 9:1859-1868. doi: 10.1105/tpc.9.10.1859 [doi] \r9/10/1859 [pii]
46. Heddad M, Adamska I. Light stress-regulated two-helix proteins in Arabidopsis thaliana related to the chlorophyll a/b-binding gene family. Proc Natl Acad Sci. 2000; 97:3741-3746. doi: 10.1073/pnas.97.7.3741
47. Jiang H-W, Liu M-J, Chen I-C, Huang C—H, Chao L-Y, Hsieh H-L. A Glutathione S-Transferase Regulated by Light and Hormones Participates in the Modulation of Arabidopsis Seedling Development. Plant Physiol. 2010; 154:1646-1658. doi: 10.1104/U.S. Plant Pat. No. 110.159152
48. Hauser F, Waadt R, Schroeder J I. Evolution of abscisic acid synthesis and signaling mechanisms. Curr Biol. Elsevier Ltd; 2011; 21: R346-R355. doi: 10.1016/j.cub.2011.03.015
49. Tuteja N, Mahajan S. Calcium signaling network in plants: an overview. Plant Signal Behav. 2007; 2:79-85. doi: 10.4161/psb.2.2.4176
50. Downton W, Loveys B R, Grant W J R. Stomatal closure fully accounts for the inhibition of photosynthesis by abscisic acid. New Phytol. 1988; 108:263-266. doi: 10.1111/j. 1469-8137.1988.tb04161.x
51. Santiago J, Rodrigues A, Saez A, Rubio S, Antoni R, Dupeux F, et al. Modulation of drought resistance by the abscisic acid receptor PYL5 through inhibition of clade A PP2Cs. Plant J. 2009; 60:575-588. doi: 10.1111/j.1365-313 X.2009.03981.x
52. Heim M A, Jakoby M, Werber M, Martin C, Weisshaar B, Bailey P C. The basic helix-52. loop-helix transcription factor family in plants: A genome-wide study of protein structure and functional diversity. Mol Biol Evol. 2003; 20:735-747. doi: 10.1093/molbev/msg088
53. Levine A, Tenhaken R, Dixon R, Lamb C. H<sub>2</sub>O<sub>2</sub> from the oxidative burst orchestrates the plant hypersensitive disease resistance response. Cell. 1994; 79:583-593.
54. Grant M, Brown I, Adams S, Knight M, Ainslie A, Mansfield J. The RPM1 plant disease resistance gene facilitates a rapid and sustained increase in cytosolic calcium that is necessary for the oxidative burst and hypersensitive cell death. Plant J. 2000; 23:441-450. doi: 10.1046/j. 1365-313 X.2000.00804.x
55. Van Horn C R, Moretti M L, Robertson R R, Segobye K, Weller S C, Young B G, et al. Glyphosate resistance in Ambrosia trifida: Part 1. Novel rapid cell death response to glyphosate. Pest Manag Sci. 2017; doi: 10.1002/ps.4567
56. Moretti M L, Van Horn C R, Robertson R, Segobye K, Weller S C, Young B G, et al. Glyphosate resistance in Ambrosia trifida: Part 2. Rapid response physiology and non-target-site resistance. Pest Manag Sci. 2017; doi: 10.1002/ps.4569
57. Gressel J. Evolving understanding of the evolution of herbicide resistance. Pest Manag Sci. 2009; 65:1164-1173. doi: 10.1002/ps.1842
58. Lunn J E, Delorge I, Figueroa C M, Van Dijck P, Stitt M. Trehalose metabolism in plants. Plant J. 2014; 79:544-567. doi: 10.1111/tpj. 12509
59. Crowe J J, Crowe L M, Chapman D. Preservation of Membranes in Anhydrobiotic Organisms: The Role of Trehalose. Science (80-). 1984; 223:701-703.
60. Luo Y, Li W M, Wang W. Trehalose: Protector of antioxidant enzymes or reactive oxygen species scavenger under heat stress? Environ Exp Bot. 2008; 63:378-384. doi: 10.1016/j.envexpbot.2007.11.016
61. Ali Q, Ashraf M. Induction of drought tolerance in maize (Zea mays L.) due to exogenous application of trehalose: Growth, Photosynthesis, Water Relations and Oxidative Defence Mechanism. J Agron Crop Sci. 2011; 197:258-271. doi: 10.1111/j.1439-037 X.2010.00463.x
62. Schluepmann H, Pellny T, van Dijken A, Smeekens S, Paul M. Trehalose 6-phosphate is indispensable for carbohydrate utilization and growth in Arabidopsis thaliana. Proc Natl Acad Sci USA. 2003; 100:6849-6854. doi: 10.1073/pnas. 1132018100
63. Crowe J H, Hoekstra F A, Crowe L M. Anhydrobiosis. Annu Rev Physiol. 1992; 54:579-599.
64 Mao G, Seebeck T, Schrenker D, Yu O. CYP709B3, a cytochrome P450 monooxygenase gene involved in salt tolerance in Arabidopsis thaliana. BMC Plant Biol. 2013; 13:169. doi: 10.1186/1471-2229-13-169
65. Goda H, Sawa S, Asami T, S F, Shimada Y, Yoshida S. Comprehensive Comparison of Auxin-Regulated and Brassinosteroid-Regulated Genes in Arabidopsis. Plant Physiol. 2004; 134:1555-1573. doi: 10.1104/U.S. Plant Pat. No. 103.034736
66. Messner B, Thulke O, Schäffner AR. Arabidopsis glucosyltransferases with activities toward both endogenous and xenobiotic substrates. Planta. 2003; 217:138-146. doi: 10.1007/s00425-002-0969-0
67. Dinitrochlorobenzene (Code C439). In: NCI Thesaurus [Internet]. [cited 2 Oct. 2017]. Available:
68. Robinson M D, McCarthy D J, Smyth G K. edgeR: A Bioconductor package for differential expression analysis of digital gene expression data. Bioinformatics. 2009; 26:139-140. doi: 10.1093/bioinformatics/btp616
69. McCarthy D J, Chen Y, Smyth G K. Differential expression analysis of multifactor RNA-Seq experiments with respect to biological variation. Nucleic Acids Res. 2012; 40:4288-4297. doi: 10.1093/nar/gks042
70. Duhoux A, Carrere S, Duhoux A, Delye C. Transcriptional markers enable identification of rye-grass (Lolium sp.) plants with non-target-site-based resistance to herbicides inhibiting acetolactate-synthase. Plant Sci. Elsevier Ireland Ltd; 2017; 257:22-36. doi: 10.1016/j.plantsci.2017.01.009
71. Gardin J A C, Gouzy J, Carrère S, Délye C. ALOMYbase, a resource to investigate non-target-site-based resistance to herbicides inhibiting acetolactate-synthase (ALS) in the major grass weed Alopecurus myosuroides (black-grass). BMC Genomics. BMC Genomics; 2015; 16:590. doi: 10.1186/s12864-015-1804-x
72. UniProt: the universal protein knowledgebase. Nucleic Acids Res. 2017; 45: D158-D169. doi: 10.1093/nar/gkw1152
73. Kanehisa M, Furumichi M, Tanabe M, Sato Y, Morishima K. KEGG: New perspectives on genomes, pathways, diseases and drugs. Nucleic Acids Res. 2017; 45: D353-D361. doi: 10.1093/nar/gkw1092
74. Huerta-Cepas J, Szklarczyk D, Forslund K, Cook H, Heller D, Walter M C, et al. EGGNOG 4.5: A hierarchical orthology framework with improved functional annotations for eukaryotic, prokaryotic and viral sequences. Nucleic Acids Res. 2016; 44: D286-D293. doi: 10.1093/nar/gkv1248
75. Ashburner M, Ball C A, Blake J A, Botstein D, Butler H, Cherry J M, et al. Gene Ontology: Tool for The Unification of Biology. Nat Genet. 2000; 25:25-29. doi: 10.1038/75556

### Example 3—Multiple Herbicide Resistance in Echinochloa colona: A Multi-Herbicide Comparative Transcriptome Analysis

The Echinochloa genera are among the most problematic weeds in upland and lowland agricultural environments throughout the world. A history of co-evolution and management with major crops, in particular rice, have led them to their modern prominence. It is their ability to adapt to both abiotic and biotic stressors that allow them to persist and accumulate the necessary genomic and physiological components to persist in dynamic agricultural environments. Our research into a multiple-resistant E. colona population from Arkansas (ECO-R) has led to the identification of several potential genomic components and physiological factors that endow high levels of resistance to propanil and quinclorac. The following research provides the first multi-herbicide comparison of the response of E. colona to cyhalofop, glufosinate, propanil, and quinclorac to describe the global transcriptional patterns. Initial investigation into the responses of ECO-R following cyhalofop and glufosinate treatment revealed the constitutive induction of both herbicide targets-acetyl COA carboxylase and glutamine synthetase, respectively. Cross response comparisons between the herbicides of interest in susceptible E. colona (ECO-S) indicate that the abiotic stress response pathway, specifically actions mediated by abscisic acid, are involved in the herbicide response. Biotic stress signaling is also key to the response by ECO-S as the accumulation of several enzymes responsible for reducing disease or pathogen infection are induced. ECO-R is very different in that it enters a state of static action following treatment, with very few genes induced across all of the herbicide responses. UGT75D1 is the only gene expressed across all of the herbicides of interest. Given its's actions as a glycosyltransferase it is possible it can interact with the four herbicides. This research validates previously held conceptions that there are shared responses following herbicide action with both abiotic and biotic stress responses. It also provides insight into the potential of a shared herbicide resistance mechanism endowing multiple-resistance.

Weeds are the most problematic biotic factors that impact crop production and threaten sustainability of modern agriculture. Crop competition with weedy species account for 34% yield loss across agricultural systems world-wide and without chemical control may result in as much 74% and 82% yield loss in major commodities such as corn and soybean, respectively, in North America [1-3]. To manage weeds in agroecosystems, herbicides are the most efficient, cost-effective tools. The efficiency and low cost of herbicides have resulted in overdependence on them as primary means for weed control [4]. The shift away from integrated approaches to a system of heavy reliance on herbicides has led to rampant herbicide resistance evolution across 91 cropping systems in over 259 weedy species [5,6]. Investigation into the underlying mechanisms of resistance has been a major topic of weed research since the late 1990s [7]. Technology advancements have expanded the capabilities of investigators and explorations on genomic approaches to understand weedy traits, including herbicide resistance, has increased significantly [4, 8, 9].

Weedy species can adapt to adverse conditions. Domestication of weedy species as crops has positioned several weeds to be less responsive to management imposed in the cropping systems that they infest. Barnyardgrass (Echinochloa crus-galli), a major rice weed, was grown and processed alongside rice 10,000 years ago in China [10]. Selection may be imposed by management strategies including tillage, modified cultural practices such as crop rotations, and herbicides which all can result in rapid evolutionary change [7,11]. The selection may lead to weedy populations arising from cultivated crops. De-domestication has occurred in California rice production, resulting in a resurgence of several weedy rice populations [12]. This evolutionary ‘escape-to-ferality’ poses a significant threat to the crop due to a lack of adequate control measures for the evolved weedy relative [12]. Upland weeds such as Palmer amaranth (Amaranthus palmeri S. Wats) also may exhibit morphological changes which aid in reproduction, driven by the cropping system for which it is grown [13]. This has led to dramatic shifts in its reproductive potential in diverse cropping conditions and expansion of the geographic range it may impact. Adaptation to control and environment have led to crop mimicry which has been observed within the Echinochloa genus [14]. Several populations of Echinochloa have been unintentionally selected for which have morphological and biological similarity to rice, making hand weeding and eradication impossible.

The Echinochloa species complex is a global concern impacting many agricultural commodities, particularly those in lowland agriculture production such as rice. These species also impact upland cropping systems with populations throughout North America observed in several grain crops, soybeans, vegetables, and perennial fruits [15]. Echinochloa is composed of several weedy species including E. colona (junglerice), E. crus-galli (barnyardgrass), E. oryzoides (early-watergrass), and E. phyllopogon (late-watergrass). In Arkansas, and throughout the Mid-south USA, the dominant species is E. colona with E. crus-galli and E. muricata also being present and growing within the same production areas [16]. While the frequency of herbicide resistance among this population does not appear to shift in favor of one species over another, their underlying genetics and biology make the Echinochloa species adaptive and problematic. Echinochloa species range in ploidy from 4× to 6×, amplifying the complexity of the genome. Management of this species has historically been a combination of cultural management and herbicides [17]. Propanil, a photosystem II inhibitor, and quinclorac, a plant growth regulator, have long been standards for Echinochloa management in the Mid-south where they have been used on an extensive number of acres. Since the early 1990s, herbicide resistance in Echinochloa in the US has been a problem with populations resistant to all major rice herbicides including propanil, quinclorac, cyhalofop, clomazone, and imazethapyr and non-rice herbicides such as glyphosate [6]. Echinochloa is described as one of the “worst herbicide-resistant” weeds in the world due to its high genetic variability, partially imparted by its ploidy [18]. This is exemplified by E. colona with 25 reported cases of resistance to 6 herbicide modes of action in 14 countries [6]. Multiple resistance is also a concern with as much as 27% of Arkansas populations exhibiting resistance to two or more herbicides, and increasing in recent years [16]. Research into the mechanisms of resistance has been limited to traditional physiological and biochemical assessment focused on single resistance mechanisms, but genomic characterization is limited. Given its unique physiology, genetics, and ability to adapt to adversity, Echinochloa should be considered as a valuable resource for information on weedy traits and herbicide resistance mechanisms.

Herbicide resistance is an adaptive response to abiotic stress. The rate of resistance evolution is dictated by several traits including fitness, fecundity, frequency of herbicide resistance genes, and the total number of individuals treated over time [18]. The underlying mechanisms of resistance, target-site or non-target-site, evolve as response to the herbicide dose. Target-site resistance is a monogenic trait resulting from mutations in the genetic code that substantially alter the herbicide target protein and reduce the herbicide activity, evolutionarily driven by high dose selection [19]. Conversely non-target-site resistance, a polygenic trait, is a result of continuous low dose selection and involves enzymes or proteins that have a role in physiological response to stress that reduce the activity or concentration of the herbicide at its target [19-21]. These processes may involve restricting the movement of, redistributing, modifying, or sequestering the herbicide. The basis for these activities are a function of the physiological processes of xenobiotic detoxification which include the four phase degradation process involving the breakdown, conjugation, transportation, and inactivation of compounds [22]. Non-target-site resistance is a complex trait that is widespread but less understood. Multiple resistance arising from one, or a combination of TSR and NTSR is the largest concern for weed management. Multiple resistance is the evolved resistance to more than one herbicide mode of action within a single plant. Non-target-site resistance has the potential to impart multiple resistance via a single mechanism which limits the options available for weed management [23].

Genomic assessment of weedy species is limited because of a lack of resources to evaluate non-model organisms. However, as the cost of ‘omics’ technologies has declined with time, a call for more genomic resources in weed science has been made [8, 24-26]. Next-generation-sequencing and bioinformatics facilitate assembly of databases that contain useful genes for various research needs and comparative analysis. The de novo transcriptome constructed from Illinois Amaranthus tuberculatus (waterhemp) populations provided the first set of herbicide-target genes for this genera and also allowed for phylogenetic assessment of other weedy species from this genus [27]. Using transcriptomics, non-target-site resistance markers for Lolium sp, have been characterized related to acetolactate-synthase (ALS) inhibitor resistance [28]. Alopecurus myosuroides (black-grass) sequencing has also led to the development of a database of non-target-site alleles for investigation into novel resistance traits [29]. This research, and others like it, have led to the development of a list of candidate genes or gene families involved in herbicide resistance including: ABC transporter, cytochrome P450 enzymes, glucosyltransferases (GT), glutathione-s-transferases (GST), among several other degradative genes [30]. RNA-sequencing was used to probe Lolium rigidum populations from Australia to elucidate potential herbicide resistance mechanisms [31]. This research identified four candidate genes-two cytochrome P450s, a nitronate monoxygenase, and one GT enzyme, with active roles in the resistant phenotype. We recently released the first de novo transcriptome of E. colona and used this to characterize herbicide response and identify potential genes involved in resistance to quinclorac and propanil [32,33]. These are the first such characterization and global genetic network characterization of the potential resistance mechanisms and co-evolved abiotic stress responsive genes.

To date, the global molecular response to herbicides in E. colona has not yet been explored. Most especially, a comparative analysis of molecular response to different herbicides has not yet been done. This research presents a comparative analysis of the transcriptome profiles of multiple-resistant E. colona in response to four herbicides. The goal is to utilize the multiple-herbicide-response-transcriptome to resolve the underlying mechanisms that could impart resistance to multiple herbicides in E. colona, or other weeds. In this study, we aimed to identify candidate genes, gene networks, and biochemical pathway modifications in a multiple-resistant E. colona that are specifically or universally responsive to cyhalofop, glufosinate, propanil, and quinclorac. The outcomes of this research will provide potential genes for non-target-site resistance and also indicate future research avenues to preemptively manage weedy populations and identify weediness traits.

### Results

### Unique Transcriptomic Profiles for the Constitutive- and Herbicide Response Differences Amongst ECO-R and ECO-S

Constitutive gene expression differences between ECO-R and ECO-S without herbicide application. Constitutive expression of gene networks and specific genes unique to ECO-R and ECO-S are presented in Rouse et al. [32]. This analysis revealed several traits unique to ECO-R that potentially predispose the population to tolerate or avoid herbicide action. The trehalose metabolism pathway was enriched in ECO-R. The constitutively upregulated processes include response to nitrate, proline catabolismo glutamate, ethylene activated signaling, response to herbicides, and trehalose metabolism in response to stress. Nitrate metabolism was also enriched, indicated by the assimilation and transport of nitrate within the plant. Among these terms were multiple highly enriched terms relating to galactinol-galactosyltransferase activity, cytoplasmic translation, transcription, transcriptional elongation and protein folding. When paired with the cellular component terms related to ribosomes, it is apparent that ECO-R exhibits elevated biological function without the addition of abiotic stress.

Using ontological terms for probing into specific genes of importance within the nontreated ECO-R treatments (ECO-R-N) revealed that ECO-R is possesses several traits that make it a more vigorous plant when compared with ECO-S-N. The comparative profile for ECO—R—N included induction of transcripts for photosynthetic apparatus proteins such as ferredoxin, ATP synthase subunits, and photosystem II core complex proteins. Enhanced photosynthetic capacity results in the potential build-up of energy sources for physiological functions. Carbon assimilation gene transcripts were also enhanced. Transcriptome profiling identified key components such as malate dehydrogenase, phosphoenolpyruvate carboxylase kinase, and pyruvate dehydrogenase subunits that were enhanced in ECO-R-N. Fatty acid synthesis, via the acetyl-CoA carboxylase transcripts for the respective proteins, was also enhanced. The constitutive induction of these genes supports many processes including trehalose biosynthesis. Gene transcripts for proteins including the synthase and phosphatase genes necessary for UDP-glucose conversion to trehalose were enhanced in ECO-R-N. This is highly relevant to this research because of the role of trehalose sugars in abiotic stress mediation. The analysis also identified several xenobiotic detoxification genes in the cytochrome P450, glutathione-S-transferase (GST), and glucosyltransferase (GT) families of proteins that may have a role in reducing exogenous compounds, like herbicides. Collectively, the induction of these genes and processes indicates that ECO-R-N may have the traits necessary, prior to herbicide action, to tolerate adversity following herbicide treatment.

Cybalofop transcriptome response following treatment. Cyhalofop elicited the same transcriptomic response in ECO-S and ECO-R (data not presented). For ECO-S, several superclusters for biological process terms composed of response to high light intensity, polysaccharide metabolism, and carbohydrate metabolism were enriched. Molecular functions including oxidoreductase activity, alternative oxidase activity, and indole acetic acid (IAA) carboxyl methyltransferase activity was also enriched. Unique to ECO-R response were 38 terms for biological processes containing superclusters for ‘response to cytokinin’, containing the terms response to stress, nitric oxide, and salt stress. Superclusters of terms relating to chitin catabolismchaperone-mediated protein folding, and ribosomal small subunit assembly were also enriched. In terms of gene expression, regardless of phenotype, a large number of plant growth activities were repressed including several for photosynthetic components, fatty acid metabolism, and nitrogen metabolism. In ECO-S, respiration related gene transcripts were induced including succinate dehydrogenase subunits, cytochrome c oxidase subunits, and cytochrome b-cl complex subunits. In terms of stress response, aminocyclopropane-1-carboxylate (ACC) synthase and oxidase enzymes were induced following treatment, leading to stress induced ethylene production. A total of 240 xenobiotic-modifying genes were induced in both ECO-S and ECO-R which included cytochrome P450s, glucosidases, GSTs, and GTs; most of which were common in both accessions. Without any comparison to the other herbicides of interest there appears to be no significant induction of genes that leads to the resistance profile to cyhalofop.
Glufosinate transcriptome response following treatment. Glufosinate response in ECO-S was primarily grouped into several ontological responses including response to fungus, flavonoid biosynthesis, amino acid import, oxalate metabolism, and aromatic compound metabolism. The glufosinate response in ECO-R was also similar to ECO-S but included several superclusters formed for the biological processes-response to xenobiotic stimulus, oxalate metabolism, glutathione metabolism, amino acid transport, hydrogen peroxide catabolismauxin biosynthesis, and protein phosphorylation. In general, photosynthesis was repressed given the number of transcripts for various components of the process. Carbon metabolism genes were largely repressed in ECO-S but induced in ECO-R. Fatty acid biosynthesis was also repressed as indicated by the decrease in transcripts for acetyl-CoA carboxylase. Glutamine synthetase, the target for glufosinate, was repressed in ECO-S. In ECO-R, one form of glutamine synthetase (7.5) and glutamine synthetase cytosolic isozyme 1-3 (8.2) were induced, indicating that ECO-R may be able to express the necessary enzyme for normal function even in the presence of the herbicide. Nitrate reductase, in multiple forms, was repressed in ECO-R, but not observed in response to glufosinate in ECO-S. As observed in ECO-S and ECO-R following cyhalofop treatment, a high number of xenobiotic detoxifying genes (666) were expressed within both accessions. Clearly, both ECO-S and ECO-R are attempting to reduce the activity of glufosinate through detoxification but no single gene can be considered the primary enzyme endowing the resistance. Further assessment using the comparative analysis is required.
Propanil transcriptome response following treatment. A detailed analysis of the propanil response transcriptome can be found in Rouse et al. [33]. Propanil enriched biological processes included response to water deprivation, abscisic acid (ABA) metabolism, maltose biosynthesis, high affinity potassium ion transport activity, and positive regulation of transcription factor catabolismolecular function terms were categorized as beta-amylase activity, transcription factor activity, hydroperoxide dehydratase activity, and galactinol-sucrose galactosyltransferase activity. Only three terms were enriched within ECO-R—P, one biological process-flavonoid biosynthesis and two molecular function terms quercetin O-glucosyltransferases. Following propanil application ECO-S induces several abiotic and biotic stress responses in an attempt to mediate the herbicide action. This includes several gene transcripts for ABA induction and metabolism. In terms of biotic responses, the jasmonic acid pathway is induced in both ECO-S and ECO-R, leading to a down-stream build-up in glucosinolates that would not have action on herbicides, only insects. Several genes associated with hypersensitive response were induced in ECO-R, which have the potential to restrict the movement and immediate action of the herbicide. This was related to the aforementioned potential build-up of trehalose from constitutive gene expression. The trehalose sugar would be beneficial following the hypersensitive response to regenerate the plant. Following treatment, induction of trehalose biosynthesis genes were also observed, further implicating the potential abiotic stress alleviation imparted by trehalose. The primary mechanisms believe to endow propanil resistance involves a two-phase process. First hydroxylation via two cytochrome P450 enzymes-CYP709B2 and CYP72A15, followed by the conjugation of the two products via the GSTU17 and an undetermined glycosyltransferase.
Quinclorac transcriptome response following treatment. A detailed characterization of ECO-S and ECO-R transcriptome following quinclorac treatment is described in Rouse et al. [32]. Gene ontology analysis for ECO-S-Q response was composed of several terms coined as auxin catabolismprotein auto-phosphorylation, and aerobic respiration. No terms were enriched following quinclorac treatment in ECO-R. In both ECO-S and ECO-R, the transcriptome response validated previous research implicating the ethylene biosynthetic pathway induction following treatment [34]. However, based on the transcriptome of ECO-S, the high expression of genes in this pathway might have occurred much earlier and by 24-hours after treatment these processes were already being repressed. This was related to the presence of ABA hydroxylase genes, which were induced in response to the concomitant induction of ABA from this same pathway. The enzyme responsible for ABA induction, 9-cis-epoxycarotenoid dioxygenase (NECD), was also being repressed 24 h after treatment due to feedback inhibition from the high ABA concentration. In ECO-R, induction of the ethylene biosynthetic pathway had stopped by 24-hours after treatment, indicating that the herbicide reached its target enzyme but did not cause rampant ethylene production. Several xenobiotic detoxification genes were induced in ECO-R-Q. One gene in particular, UGT75D1, was identified that is potentially involved in conjugating the active quinclorac molecule. Another gene of interest in the ECO-R-Q transcriptome was one that codes for ALPL1 protein, a potential epigenetic factor that antagonizes polycomb group proteins that repress DNA transcription. ALPL1 may be what is allowing the high expression of UGT75D1 in ECO-R. Again, the role of constitutive induction of trebalose biosynthesis was also described to play a large role in mitigating the secondary or tertiary effects of quinclorac.

### Functional Characterization of the Herbicide Response Transcriptome of ECO-S

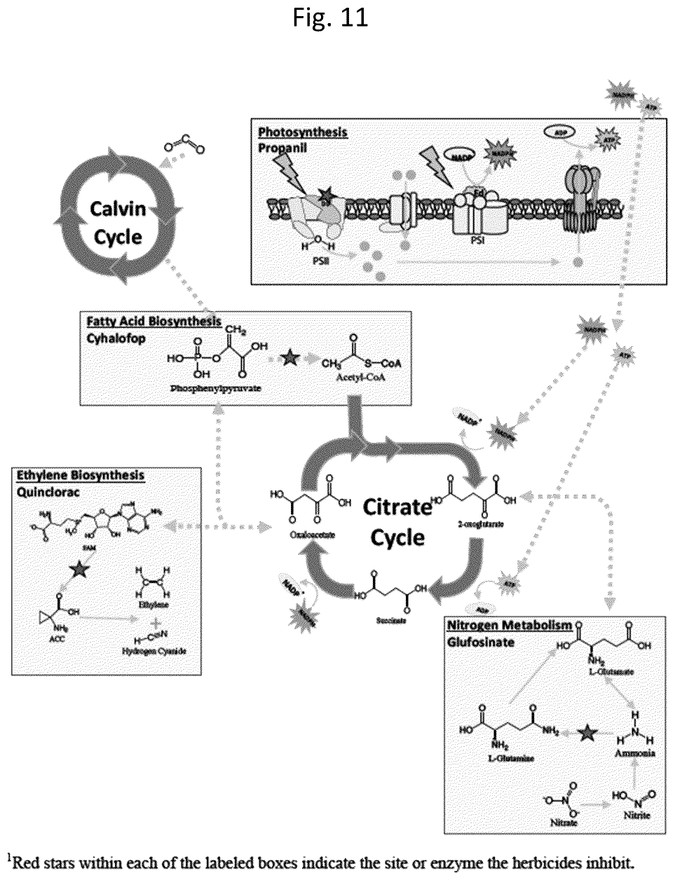

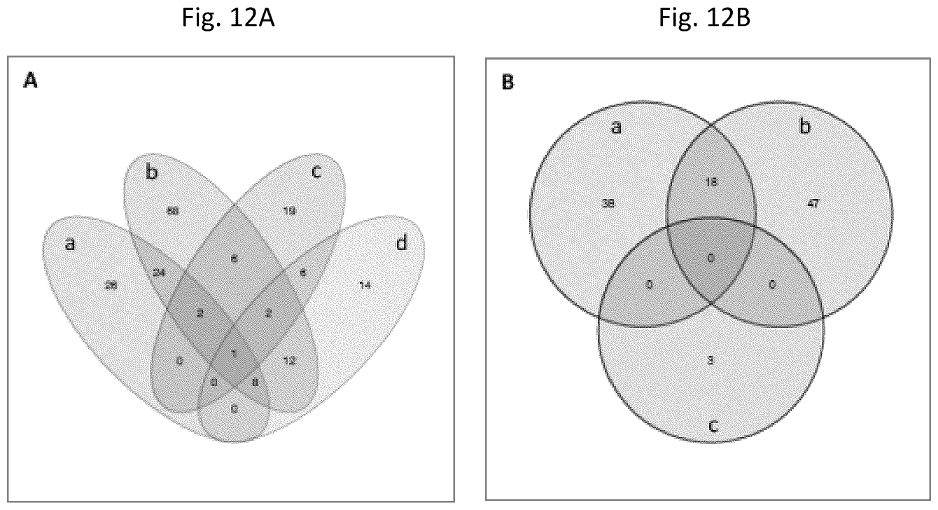

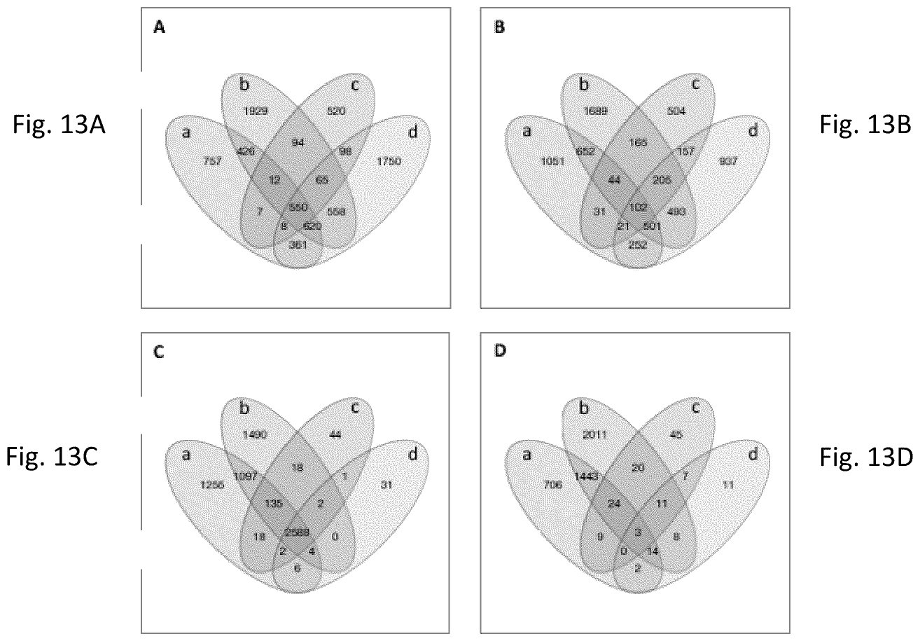

Gene ontology enrichment in ECO-S across herbicide treatments. A comparative analysis for enriched terms related to herbicide response across all four herbicides was conducted (FIG. 12A). A total of 188 terms were enriched following herbicide treatment in ECO-S. Unique terms to each herbicide include 26 for cyhalofop, 68 for glufosinate, 19 for propanil, and 14 for quinclorac. Sixty-one enriched processes across all four herbicides were shared amongst two or more of the transcriptome profiles (FIG. 12A). Only one, inositol oxygenase induction, was shared by all four herbicide response profiles. Two enriched terms were shared between cyhalofop, glufosinate, and propanil treatments in the susceptible phenotype (ECO-S-C/G/P): inositol catabolic process and response to karrikin (FIG. 12A). Nitrate assimilation and salicylic acid catabolic processes were enriched across glufosinate, propanil, and quinclorac (ECO-S-G/P/Q). The terms shared between cyhalofop, glufosinate, and quinclorac (ECO-S-C/G/Q) included several biological processes categorized as oxidation-reduction process, response to oxidative stress, and hydrogen peroxide metabolism as well as the molecular function terms heme-binding and peroxidase activity. Responses shared only between cyhalofop and glufosinate (ECO-S-C/G) included the biological processes categorized collectively as response to ABA, killing of cells of other organisms, toxin catabolismglutathione metabolism, and ABA biosynthesis. Three biological process and three molecular function terms were shared between glufosinate (ECO-S-G) and propanil (ECO-S-P) responses; all were related to nitrate transport and trehalose biosynthesis. Given the sites of action and biological pathways associated with propanil and quinclorac (FIG. 11) several shared terms related to ABA catabolismand signaling, as well as ethylene-activated signaling were enriched as expected. Responses common between glufosinate and quinclorac (ECO-S-G/Q) included cell surface receptor signaling, defense response to oomycetes, and response to bacterium, categorized as response to jasmonic acid. Several molecular function terms were also enriched-ATP binding, oxidoreductase activity, polysaccharide binding, symporter activity, and protein kinase activity. Collectively, regardless of the herbicide used, it is apparent that the susceptible plants perceived the abiotic stress caused by the herbicides and responded accordingly within 24 hours. While each herbicide has a unique physiological effect, the superclusters of terms affected by all four herbicides were related to only a few endogenous hormones (ABA, salycilic acid, auxin, jasmonic acid) or compounds (sugars or carbohydrates). It was also clear that signaling to specific biological pathways was active; hence, the enrichment or expression of several similar processes across the four herbicide treatments.
Expression of genes related to plant growth and maintenance proteins. A total of 22,761 transcripts were differentially expressed in ECO-S amongst the four herbicide treatments (FIGS. 13A-13B). For the respective transcriptome profiles, the numbers of differentially expressed transcripts were 5,395 with cyhalofop, 8,105 with glufosinate, 2,583 with propanil, and 6,678 with quinclorac. In general, the number of repressed genes was similar to the number induced by the herbicides. Five hundred and fifty transcripts were repressed and shared amongst all four herbicides of interest in ECO-S (FIG. 13A). The repressed genes are primarily associated with a reduction in biological activity and a shift to abiotic stress response. Collectively, these genes, which include ribosomal proteins, kinases, cytoskeletal related proteins, elongation factors, RNA polymerases, and several ATP related enzymes are involved in maintenance and plant growth and development. Thus, the plant's initial response is to repress growth proteins and produce only what is necessary for sustaining minimum-level processes under abiotic stress.
Expression of genes related to abiotic and biotic stress response characterization. The induction of 102 transcripts, common to four herbicide treatments, suggested a shift from plant growth to a state of survival. Several abiotic stress-induced genes were expressed (FIG. 13B). ABA receptor PYL5, was induced (2.6 to 5.3) conferring perception of elevated ABA, leading to stomatal closure [35]. The transcription factor MYB44 is also induced by the four herbicides because of the ABA induction and enhances the abiotic stress tolerance via the action of stomatal closure [36]. While stomatal closure helps to alleviate immediate stress it also results in the production of reactive oxygen species (ROS). This is caused an increase in the electrons in the transfer chain without the necessary CO2 concentrations to complete the assimilation process. Three peroxidase genes with known ROS reductive properties were induced following treatment across herbicides: PER15, PER54, and PER57. Stress induced genes in the ethylene biosynthetic pathway were not expressed; however, shared downstream responses indicate the presence of ethylene. Three ethylene responsive transcription factors (ERTF) were expressed ERF073, ERF113, and RAP2-1. These three are all involved in transcriptional activation and bind to the GCC-box pathogenesis promoter; ERF113 is known to be induced by wounding and waterlogging [37]. These, along with several pathogen-response-related genes, were also induced by the herbicide treatments. Forty-eight forms of indole-3-acetaldehyde oxidase transcripts, the majority in the cyhalofop (21) and glufosinate (22) responses were induced. In response to pathogen infection, indole-3-acetaldehyde oxidase can produce auxin and hydrogen peroxide-a stress signal, protective, and potentially harmful molecule [38,39]. Mitogen-activated protein (MAP) kinase 8 and MAP kinase kinase 5 were induced. Both are essential for host-immune response in the pathogen defense pathway, but also induced by oxidative stress and high light intensity, resulting in reduction of ROS [40-42]. Finally, two genes that could potentially interact with herbicides were also induced-ABCG53 and UGT74D1. ABCG53 is listed as a possible defense protein. UGT74D1 conjugates IAA rendering it inactive; this may serve as a feedback response to the IAA oxidase, which forms the active IAA molecule. Given the susceptibility of this population to the herbicides, the induction of these detoxifying enzymes does not impart resistance to herbicides and may be produces to mitigate some of the secondary herbicide effects on the plant physiology.

### Functional Characterization of the Herbicide Response Transcriptome in ECO-R

Gene Ontology enrichment in ECO-R following herbicide treatment. Enrichment of ontological terms following treatment in ECO-R was much lower than in the ECO-S population. In total, 108 terms were significantly enriched, with only 18 shared between ECO-R—C and ECO-R-G responses (FIG. 12B). For the 18 shared terms between ECO-R—C and ECO-R-G, the majority of the biological processes were linked to RNA translation and protein synthesis, similar to the molecular function terms. While the data for gene ontology depletion is not presented, no depletion in response to propanil nor quinclorac were observed. Only a few terms were depleted by cyhalofop and glufosinate treatment and were mostly related to protein synthesis. These ontological enrichment profiles were a stark contrast to the ECO-S population. ECO-R appears unresponsive to the herbicide treatments. Another factor of note in the response is the relatively low enrichment and lack of depletion in ECO-R before and after treatment. This would indicate that the ontology terms that were constitutively enriched in ECO-R, were also present to some degree following treatment. No changes in carbohydrate partitioning nor enhanced metabolism are evident given the herbicide treatment responses in ECO-R.
Repression of major plant growth and maintenance gene transcripts. The differential expression of transcripts in ECO-R across the herbicide responses was similar to ECO-S with a total of 21,791 transcripts characterized (FIGS. 13C-13D). However, unlike ECO-S, the vast majority (73%) of differentially expressed transcripts in ECO-R were repressed following herbicide treatment (FIG. 13C). Collectively 2,591 transcripts were shared across the four herbicide responses; 2,588 of these were repressed while only 3 were induced. As observed in ECO-S, a number of growth related genes are repressed following treatment including a large number of ribosomal proteins-40S and 60S, limiting protein synthesis. DNA-related proteins including several DNA repair proteins, topoisomerases, and polymerases enzymes and proteins were also repressed. This occurred along with the depression in endoplasmic reticulum (ER)-related genes ER lectin 1, ER oxidoreuctin-1 and ER-golgi intermediate compartment 3. Several ATP-related enzymes including mitochondrial and chloroplastidic ATP synthase subunits, ATP-binding cassettes, ATP-dependent RNA helicases, ADP/ATP carrier proteins were repressed. Other repressed purine-related enzymes include AMP deaminase, ADP ribosylation factor-like proteins, and ADP-sugar pyrophosphatase. Examination of genes associated with the constitutively enhanced pathways revealed repression of most genes related to carbon metabolism. These include aspartate aminotransferase, PEP carboxylase, malate dehydrogenase, and NADP-dependent malic enzyme. At the same time, several sugar-metabolism-related genes including GDP-L-fucose synthase, GDP-mannose 4,6 dehydratase 2, GDP-mannose transporter, and several glucose transporters-1E and 2A were repressed. None of the trehalose-related enzymes were repressed across all four herbicide treatments. However, alpha-alpha trehalose phosphorylase, which is responsible for the breakdown of trehalose into D-glucose, was repressed approximately seven-fold across all responses. A putative reduction in fatty acid synthesis was observed based on reduction in transcripts for acyl-CoA related enzymes including a dehydrogenase, desaturase, synthetase, and binding domains. Acetyl-coenzyme A synthetase is required for the formation of acetyl-CoA, this enzyme was repressed following treatment. The ACCase and ACCase 2, which were constitutively enhanced, were repressed across the four herbicide treatments along with several very long chain fatty acid elongation proteins 2 and 6. The ACCase enzymes were not the only herbicide-response related genes repressed across treatments. Several forms of the glutamine synthetase enzyme, inhibited by glufosinate, were repressed by as much as 10.5-fold across all herbicide treatments. In general, repression of most genes was also related to plant growth and development as observed in ECO-S. Several critical pathways were repressed including the carbon metabolism, energy relations, nitrate assimilation, and fatty acid synthesis pathways, providing evidence that ECO-R is preserving previously formed energy sources and minimizing the destructive impact of herbicides or abiotic stressors. Induction of xenobiotic-related gene transcripts. Unlike ECO-S, only three transcripts were induced across all four herbicides in ECO-R: protein ALP-1 like protein, secologanin synthase, and UGT75D1 (FIG. 13B). Across the four responses no large-scale shift toward stabilizing or maintenance proteins occurred that would indicate a state of significant abiotic stress as seen in ECO-S. Multiple herbicide detoxification genes were commonly induced across three herbicide treatments. GSTU17, GST23, and disease resistance protein RPMI were all expressed across transcriptomes of ECO-R-C/G/P. For ECO-R-G/P/Q comparisons-CYP89A2, CYP709B2, CYP709B1, UGT73E1, and UGT73D1 were induced. Finally, ECO-R-C/G/Q had no herbicide detoxification genes shared across the responses. Only CYP709B1 was not expressed in ECO-S following any one of the four treatments meaning this enzyme may have implications for resistance. CYP709B2 was however induced across ECO-S-C/G/Q, but not in ECO-S—P, further supporting its potential role in herbicide detoxification, except with propanil. None of these CYP genes were expressed in ECO-S-P even though they were induced in at least one other herbicide response in ECO-S. Overall, this shift in favor of repression in ECO-R following treatment is opposite of what occurred in ECO-S whereby the reduction in transcripts was paired with an almost equal induction of stress-responsive genes. Far fewer genes were induced across all herbicide treatments in ECO-R. The resistant plant appeared to be in a stasis condition with minimal biological activity and far less response to abiotic stress stimuli compared to ECO-S.

### Comparative Differential Gene Response and Functional Characterization Between ECO-S and ECO-R Following Herbicide Application

A total of 2004 transcripts, 1281 repressed and 723 enhanced, were differentially expressed in ECO-R across the four herbicides than in ECO-S. Only 44 were shared across the four herbicide responses. For the repressed transcripts, 40 were shared across the herbicides and were primarily characterized as transporters and integral membrane proteins associated with movement of solutes and other compounds into and out of the cell. Two aquaporin proteins TIP4-1 and TIP4-2, two oligopeptide transporters 2 and 4, and two nitrate transporters NPF6.2 and NPF6.3 were comparatively repressed in ECO-R. Multiple stress responsive genes were repressed in ECO-R including ABA related proteins. ABA 8′-hydroxylase 1 and ABA receptor PYL5 were both repressed in response to the treatments. E3 ubiquitin-protein ligase MIELI and XB3 and VQ motif-containing protein 25 [43], all responses to abiotic and biotic stress perception and signaling were significantly repressed. In terms of pathogen/disease response, which was significantly induced in ECO-S, two genes ERF073 and pathogen-related protein were depressed in ECO-R compared with ECO-S. Collectively the repressed genes that signify the comparative response between ECO-S and ECO-R validate the described differences in the state of the plants following treatment. ECO-S devotes a number of resources to moving solutes around the plant to supply the needed substrates for the elevated enzymatic reactions. ECO-R does not perceive an elevated abiotic stressed state and thus requires few resources.

Only three genes were enhanced across the four herbicides: nudix hydrolase 21, ERF4, and two forms of Protein ALP1-like. Nudix hydrolase 21 is a general-purpose enzyme involved in the hydrolysis of nucleoside diphosphate derivative capable of producing orthophosphate. ERF4 is a transcriptional repressor of the aforementioned GCC-box pathogenesis promoter element. Finally, protein ALP1-like, also mentioned previously, may be a stress induced antagonist of the polycomb group of genes associated with chromatin modifications in the form of transcriptional repression [44]. For cyhalofop response, only 39 transcripts were unique and among these were only a few genes potentially related to herbicide detoxification: CYP71A1, CYP71A35, CYP72A14, and CYP87A3. Glufosinate response elucidated enhancement in 81 transcripts for ECO-R; in terms of xenobiotic interactions, only CYP71A1 CYP76M5 had elevated expression. Propanil response was much higher with as many as 129 unique transcripts. CYP704C1, CYP709B2, CYP72A15, GST1, GST4, GSTU17, UGT73D1, UGT88A1, and UGT88F3 were all enhanced to a greater number following treatment in ECO-R. Finally, ECO-R-Q, elicited 122 unique transcripts, including CYP709B2, CYP45071A1, CYP71A4, CYP71A8, GSTU20, UGT73D1, UGT75D1, and UGT88A1. Few genes were shared amongst the various profiles. In general, several shared responses did include stress-induced proteins including heat stress proteins, ERFs, and ABC transporters. However, no unique profiles significantly distinguished themselves as having a causative role in resistance or stress mitigation.

### DISCUSSION

Unique Herbicide Responses have a Role in the Mitigation of Herbicide Action to Cyhalofop and Glufosinate

The resistance to cyhalofop and glufosinate in ECO-R is marginal in ECO-R compared with ECO-S. However, the transcriptome does reveal several elements, which may contribute to the observed level of resistance and may indicate the early evolutionary period of resistance in this population. The constitutive enhancement of ACCase in ECO-R implies that when treated with an ACCase inhibitor (cyhalofop), the putatively higher amount of enzymes present would reduce the inhibitory effects of the herbicide. At the plant level, this was exhibited as the ability to recover from phytotoxic effects of cyhalofop as observed in previous experiments with ECO-R [45]. After cyhalofop treatment, these are greatly repressed indicating there is still an interaction between the herbicide and its site of action. For glufosinate, the target enzyme glutamine synthetase was also enhanced prior to treatment and repressed following application, but another form of glutamine synthetase was induced It is possible that the overall mechanism driving the enhanced activities described previously, preempts the effects these herbicides have. This paired with the effects that the trehalose metabolism may have on abiotic stress tolerance may be the causal agents in reducing the effects of these herbicides.

Abiotic Stress Inducible Response is a Primary Action in Susceptible E. colona

Within 24 hours of the herbicide application, a series of physiological events including perception, signaling, and transduction of abiotic stress occur. In general, the transcriptome profile indicates that the plant induces a number of the growth and maintenance processes associated with early development to aid in the stress response. The response to each herbicide were in accordance to what is known about their respective modes of action. The transcriptome data provided mechanistic details of how certain responses to herbicides come about. A common response to all four herbicides was increased ABA perception and signaling, which was indicative of increased ABA production. ABA signaling is a key component in abiotic stress response and results in the closing of leaf stomata, limiting water transpiration and increasing free radical production [46]. ABA concentrations in the cell have downstream effects on calcium ion redistribution via the induction of calcium permeable channels, which aid in the mediation of abiotic stresses [47,48]. The concerted signals enabled by the ABA molecule and calcium ion help to mitigate the negative impact of abiotic stresses (i.e. drought, cold, heat) have on the plant. However, long-term stomatal closure due to ABA directly represses the photosynthetic process and leads to the build-up of reactive oxygen species with cell membrane damaging properties.

Biotic Stress Mitigation is Also Central to the Action of Susceptible E. colona

ABA action does provide an immediate stabilization effect, it is clear that ECO-S still induces additional biotic stress mitigating proteins and enzymes to prevent prolonged negative impacts that lead to plant death. This includes the increased expression of several transcription factor proteins associated with host-pathogen responses, hydrogen peroxide forming enzymes, and MAP kinase proteins. Collectively these processes would not be capable of detoxifying herbicides or reducing the secondary damaging effects of their action. The production of hydrogen peroxide is helpful in the host-immune response to biotic pathogens and has been described to have a potential role in propanil response. But when paired with the reactive oxygen species (ROS) evolved from the buildup of free energy in the photosynthetic electron transport chain the compounding effects may be harmful. This response to herbicide has been characterized for aiding in the suppression of Sclerotenia stem rot infection in soybeans [50]. Lactofen, a protoporphyrinogen IX inhibiting herbicide applied to soybean, is believed to induce a hypersensitive response similar to most plant defense to pathogen activity. Peroxidase genes and isoforms of these genes, capable of neutralizing the activity of ROS, were induced by as much as 11-fold in the responses to all of the herbicides. The production of indole-3-acetyhyde oxidase is also of note in the response. This leads to the production of hydrogen peroxide and is a potential response to pathogen infection; it also indicates a demand for auxin production and a build-up of the IAA precursor. Auxin, the intercellular signal molecule, is necessary to direct general plant growth [51]. However, under the stress induced state, elevated auxin production and uncontrolled accumulation of free auxin alone could result in reduced plant growth and lead to plant death as observed with auxin-type herbicides [52]. The concomitant increase in hydrogen peroxide also produced via this auxin catabolic pathway also has negative effects on the health of the plant. Finally, the repressed genes across all of the responses indicates that the shift to stress response comes at a significant cost to the developmental potential of ECO-S. The multitude of proteins reduced were seemingly related to the production of maintenance proteins necessary for cell elongation and division, as well as cytoskeletal development. Given this profile, it is clear that the survival state following treatment is a high energy demanding process that on its own limits the production potential of ECO-S. This comes at a dramatic cost to the plant, which may not be recoverable if ECO-S were to recover.

### Multiple-Herbicide Resistance May be an Adaptive Evolutionary Response to Herbicides and Abiotic Stress

Adaptive evolution may be the single most advantageous process employed by weedy species in agro-environmental landscapes. The genomic plasticity results in weedy populations existing in a middle-ground state of highly advantageous domesticated traits and strong genomic resources for exploitation of weediness [53]. ECO-R is a unique population, highly resistant to propanil and quinclorac with low level resistance to cyhalofop and glufosinate, with a tremendous ability to tolerate adversity and produce high levels of biomass (date not shown). Given the heightened state of abiotic stress exhibited by ECO-S and the repression in the plant growth proteins, it is clear that a latent effect of herbicide action is a depletion in energy reserves. This depletion paired with the continuous inhibition of key enzymes by the herbicides lead to plant death. Any process that can supply and protect the cellular structure under this stress, would benefit the plant and assist in overcoming the secondary effects of herbicide stress.

Analysis of the constitutive difference between the contrasting ECO-S and ECO-R populations revealed a litany of biological processes that are functioning at a greater level in ECO-R compared with ECO-S. Of the enhanced processes, were major proteins related to photosynthesis, carbon assimilation, fatty acid metabolism, and sugar metabolism and transport. Specifically, within these processes were a significant number of transcripts associated with the trehalose biosynthetic process-the trehalose phosphate synthase (TPS) and the trehalose phosphate phosphatase (TPP) enzymes. This has not been proposed as a potential pathway to mitigate herbicide effects previously but our research has explained the means to which this may occur. The trehalose pathway has been described in the literature due to its overwhelming positive effect on abiotic stress response and adaptive ability to oxidative and drought stressed conditions [54,55]. The presence of these key enzymes, paired with the elevated activity dedicated to growth, and the ontological enrichment of terms related to trehalose response to stress, provide overwhelming support for the role of trehalose in mitigating the herbicide stressors. This complex pathway would allow the plant to persist under the conditions imposed by herbicide activity of a variety of compounds more than just those investigated in this research would. This has the potential to mitigate the harsh effects caused by several herbicides that effect tolerance to photosynthesis inhibiting herbicides (WSSA Group 5/6/7), protoporphyrinogen IX inhibitors (WSSA Group 14), cell membrane disruptors (WSSA group 22), phytoene desaturase inhibitors (WSSA Group 12), diterpene biosynthesis inhibitors (WSSA Group 13), and HPPD inhibitors (WSSA Group 27). Based on this information, it is plausible that only propanil and quinclorac are being actively metabolized, and the cyhalofop and glufosinate resistance is imparted by the effects of this trehalose biosynthesis.

Only three stress-induced genes were shared across the herbicide responses. While UGT75D1 may have be active on the herbicides, it does not appear to impart a high level of resistance to cyhalofop, glufosinate, or propanil alone. Given their structures, it is possible that UGT75D1 will still conjugate cyhalofop and propanil in a similar manner to quinclorac but not completely inactivate the compounds. This indicates that our hypothesis of a shared resistance mechanism is not present in ECO-R. However, the continued presence of the ALPL1 protein across the various responses is of interest. The literature describes this protein as potentially stress-induced [56,57] and given that it is present in all of the response profiles, it is clearly induced within ECO-R in response to herbicide stress. The elevated presence of this protein may have a number of effects on the multiple herbicide-resistant phenotype that would require further validation. While we posed that there is a single mechanism endowing the multiple resistance through detoxification, it is possible that the role of this epigenetic repressor antagonist may be more important. This would support the idea of adaptive co-evolution of abiotic stress resistance proteins and the shared role they may have in herbicide resistance. It may also be that ALPL1 has an active role in expressing certain genes, which may be beneficial to the plant under high stress conditions or herbicide application.

Herbicide activity in plants results in a complex and genetically diverse response to abiotic stress as observed in the characterization of the ECO-S and ECO-R transcriptome presented in this research. However, using constitutive gene expression of contrasting phenotypes and supplementing this information with the transcriptomic response following herbicide application reveals a great deal of information on multiple herbicide resistance. Given the profile for ECO-S, it is apparent that herbicide stress is perceived very similarly to both biotic and abiotic stressors. The initial cascade of responses, 24 hours after treatment, relate to processes associated with reducing the effects of drought stress as well as several key components in host pathogen response, to disrupt or control a pest/pathogen. However, the induction of a variety of these genes result in the secondary or tertiary effects of herbicide action, most notably hydrogen peroxide and ROS formation. ECO-R is a much different population that has evolved to not only metabolically reduce herbicide action via xenobiotic detoxification, but has evolved to compensate these mechanisms through and enhanced carbohydrate assimilation pathway. This is the first such description of the trehalose biosynthetic process imparting tolerance to herbicides and the subsequent effects it has on mitigating the abiotic stress effects caused by herbicide action. This will require further research into the role it plays in weedy species, specifically Echinochloa, and quantifying the effects it has on abiotic stress and potentially herbicide resistance. It is also of interest that this ECO-R population is capable of shifting into a near sedentary state following herbicide application, as indicated by the vast repression of genes following treatment. This implies that by reducing the activities of the plant there can be less secondary effects, which were describe in the ECO-S population. This may be an example of a weedy species reducing biological activities to allow for the herbicide detoxification to occur, prolonging the period in which the enzymes may act and reducing secondary herbicide effects.

### Methods

### Plant Materials

Echinochloa colona samples were selected from the Arkansas state-wide sampling program conducted at the University of Arkansas between 2010 and 2011 based on their profiling in the surveys presented in Rouse et al. [16]. The herbicide susceptible (ECO-S) and multiple-resistant (ECO-R) populations were profiled previously for their respective herbicide resistance profiles and potential physiological mechanisms of resistance [45]. For the RNA-sequencing experiments pureline generated seed of both ECO-R and ECO-S were grown in isolation in a growth chamber set to a 14-hour day length, 33 C day temperature and 24 C night temperature. Approximately one week after planting the plants were thinned to a single plant per pot, with two plants per accession serving as two biological replicates. When the plants reached the 2- to 3-leaf, one collar stage, they were treated with the four respective herbicides at the field application rates listed in Table 7. An identical set of plants was prepared and left untreated to serve as a nontreated control for the experiment. Twenty-four hours after treatment, the shoots from all plants were frozen in liquid nitrogen and stored in −80 C for further processing. The tissues were transferred into individual tubes containing RNAlater™-Ice for shipping to the Clemson University Genomics Institute (CUGI) for RNA extraction and library preparation.

TABLE 7
Herbicides, trade names, application rate and
adjuvant with rate used for the RNA-sequencing
experiments conducted on ECO-R and ECO-S.

| | Trade | Application Rate | Adjuvant |
|---|---|---|---|
| Herbicide | Name | kg ha⁻¹ | % |
| cyhalofop | Clincher ® | 315 | 1% COC |
| glufosinate | Liberty ® | 590 | 0.25% NIS |
| propanil | Stam ® | 4500 | 0.25% NIS |
| quinclorac | FacetL ® | 560 | 1% COC |

### RNA Sequencing, Transcriptome Assembly, and Functional Annotation

The process for RNA extraction, sequencing, transcriptome assembly, and functional annotation are detailed in Rouse et al. [32]. A summary of these processes is presented here. Total RNA was extracted using a commercially available kit by CUGI. The prepared RNA samples for all treatments were fragmented and reverse transcribe into cDNA using random primers for library assembly. The fragments were then annealed with an additional ‘A’ and the adapter sequence for high-throughput sequencing. Following enrichment via PCR, the cDNA library was submitted to the Holdings Cancer Center at the Medical University of South Carolina, Charleston SC, USA for sequencing. The samples were arranged in three lanes on the Illumina Hiseq 2500 platform and analyzed using paired end reads.

The de novo transcriptome was assembled using all of the treatments for both the ECO-R and ECO-S accessions. The transcriptome was assembled using the Trinity RNA-Seq pipeline (Broad Institute, Cambride, MA, USA). Following the primary raw data processing the normalized reads wre assembled using Trinity with the stranded library set as the default. Transdecoder 3.0.1 (Broad Institute) was used to identify open reading frames in the transcriptome and assign proteins to the gene sequences based on homology to the blastP database and HMM scan against pfam. Transcripts matching both criteria were retained for analysis. After quality assessment of the transcriptome, the Trinotate 3.0 suite of software (trinotate) utilized the BLAST+ and Swissprot databases to generated functional annotation of the proteins. It also produced output for the eggnog, GO, and KEGG databases for each of the annotated proteins.

### Comparative Assessment

Gene ontology (GO) enrichment and differential gene expression analysis was conducted for the paired treatments of interest. Several treatment conditions were paired to assess the responses of interest including nontreated ECO-S with nontreated ECO-R, nontreated ECO-S/R with their respective herbicide treatments, and the four-herbicide treatments for ECO-S and the counterpart ECO-R treatments. For the gene ontology analysis, the Trinotate output was analyzed using the ‘goseq’ package from Bioconductor (bioconductor) to assign GO terms to the transcripts from the transcriptome. The analysis of enriched terms was performed only on transcripts, which were expressed or depressed at a log 2 fold-change of ≤-2 or >2 and a p-value of ≤0.01. The results of the analysis were visually assessed based on the p-value of the GO analysis using REVIGO (revigo) to best characterize the results and identify representative subsets and superclusters of the terms using clustering algorithms based on semantic similarity [58]. Cytoscape (Cytoscape Consortium, San Diego, CA, USA) software (cytoscape) was used to visualize the output from REVIGO to produce relevant graphics of the results.

Differential gene expression was conducted using the R statistical software program (r-project) with the Bioconductor package-edgeR [59,60]. EdgeR was used to quantify the filtered raw counts from the RNA-sequencing experiment. Standard normalization using the trimmed mean of M-value was applied to the counts and the counts were fit using a GLM model for the determination of significance. A log-fold change was determine based on these results and used to describe the expression change under the various treatment conditions. The data were visualized using volcano plots and used for the follow-up descriptive analysis to identify patterns of gene expression. Further manual processing of the differential gene expression data applied categories and gene families to the results. The JMP® Pro 13.1 (SAS Institute, Cary, North Carolina, USA) software was used to summarize the results and the Venn diagram add-in package was used to compare the various treatments and produce relevant graphs. Descriptions of the genes and the physiological pathways for which they function are based on the data in the Uniprot and KEGG databases.

### REFERENCES FOR EXAMPLE 3

1. Dille J A, Sikkema P H, Everman W., Davis V M, Burke I C. Perspectives on corn yield losses due to weeds in North America. 55th Annual Weed Science Society of America Annual Meeting. Lexington, KY: Weed Science Society of America; 2015.
2. Dille J A, Sikkema P H, Everman W., Davis V M, Burke I C. Perspective on soybean yield losses due to weeds in North America. 55th Annual Weed Science Society of America Annual Meeting. Lexington, KY; 2015.
3. Oerke E C. Crop losses to pests. J Agric Sci. 2006; 144:31-43. doi: 10.1017/S0021859605005708
4. Shaw D R. The “Wicked” Nature of the Herbicide Resistance Problem. Weed Sci. 2016; 552-558. doi: 10.1614/WS-D-15-00035.1
5. Neve P, Busi R, Renton M, Vila-Aiub M M. Expanding the eco-evolutionary context of herbicide resistance research. Pest Manag Sci. 2014; 70:1385-1393. doi: 10.1002/ps.3757
6 Heap I. International Survey of Herbicide Resistant Weeds [Internet]. 22 Aug. 2017 pp. 145-153.
7. Jordan N R, Jannink J L. Assessing the practical importance of weed evolution: a research agenda. Weed Res. 1997; 37:237-246.
8. Stewart C N, Tranel P J, Horvath D P, Anderson J V., Rieseberg L H, Westwood J H, et al. Evolution of Weediness and Invasiveness: Charting the Course for Weed Genomics. Weed Sci. 2009; 57:451-462. doi: 10.1614/WS-09-011.1
9. Hall J C, Van Eerd L L, Miller S D, Owen M D K, Prather T S, Shaner D L, et al. Future Research Directions for Weed Science. Weed Technol. 2000; 14:647-658. doi: 10.1614/0890-037 X (2000) 014 [0647: FRDFWS] 2.0.CO; 2
10. Yang X, Fuller D Q, Huan X, Perry L, Li Q, Li Z, et al. Barnyard grasses were processed with rice around 10000 years ago. Sci Rep. Nature Publishing Group; 2015; 5:16251. doi: 10.1038/srep16251
11. Clements D R, Ditommaso A, Jordan N, Booth B D, Cardina J, Doohan D, et al. Adaptability of plants invading North American cropland. 2004; 104:379-398. doi: 10.1016/j.agee.2004.03.003
12. Kanapeckas K L, Vigueira C C, Ortiz A, Gettler K A, Burgos N R, Fischer A J, et al. Escape to ferality: The endoferal origin of weedy rice from crop rice through de-domestication. PLOS One. 2016; 11:1-23. doi: 10.1371/journal.pone.0162676
13. Bravo W, Leon R G, Ferrell J A, Mulvaney M J, Wood C W. Differentiation of Life-History Traits among Palmer Amaranth Populations (Amaranthus palmeri) and Its Relation to Cropping Systems and Glyphosate Sensitivity. Weed Sci. 2017; 65:339-349. doi: 10.1017/wsc.2017.14
14. Barrett S H. Crop mimicry in weeds. Econ Bot. 1983; 37:255-282. doi: 10.1007/BF02858881
15. Van Wychen L. 2015 Baseline Survey of Most Common and Troublesome Weeds in the United States and Canada. In: Weed Science Society of American National Weed Survey Dataset [Internet]. 2015 [cited 22 Mar. 2017].
16. Rouse C E, Burgos N R, Norsworthy J K, Tseng™, Starkey C. Echinochloa resistance to herbicides continues to increase in Arkansas rice fields. Weed Technol. 2017; In Press.
17. Talbert R E, Burgos N R. History and Management of Herbicide-resistant Barnyardgrass (Echinochloa Crus-galli) in Arkansas Rice. Weed Technol. 2007; 21:324-331. doi: 10.1614/WT-06-084.1
18. Heap I. Global perspective of herbicide-resistant weeds. Pest Manag Sci. 2014; 70:1306-1315. doi: 10.1002/ps.3696
19. Gardner S N, Gressel J, Mangel M. A revolving dose strategy to delay the evolution of both quantitative vs major monogene resistances to pesticides and drugs. Int J Pest Manag. 1998; 44:161-180. doi: 10.1080/096708798228275
20. Délye C. Unravelling the genetic bases of non-target-site-based resistance (NTSR) to herbicides: A major challenge for weed science in the forthcoming decade. Pest Manag Sci. 2013; 69:176-187. doi: 10.1002/ps.3318
21. Yuan J S, Tranel P J, Stewart C N. Non-target-site herbicide resistance: a family business. Trends Plant Sci. 2007; 12:6-13. doi: 10.1016/j.tplants.2006.11.001
22. Kreuz K, Tommasini R, Martinoia E. Old Enzymes for a New Job. Plant Physiol. 1996; 349-353.
23. Yu Q, Powles S. Metabolism-Based Herbicide Resistance and Cross-Resistance in Crop Weeds: A Threat to Herbicide Sustainability and Global Crop Production. Plant Physiol. 2014; 166:1106-1118. doi: 10.1104/U.S. Plant Pat. No. 114.242750
24. Zhang Q, Riechers D E. Proteomics: An Emerging Technology for Weed Science Research. Weed Sci. 2008; 56:306-313. doi: 10.1614/WS-07-089.1
25. Shaner D L, Beckie H J. The future for weed control and technology. Pest Manag Sci. 2014; 70:1329-1339. doi: 10.1002/ps.3706
26. Basu C, Halfhill M D, Mueller T C, Stewart C N. Weed genomics: New tools to understand weed biology. Trends Plant Sci. 2004; 9:391-398. doi: 10.1016/j.tplants.2004.06.003
27. Riggins C W, Peng Y, Stewart C N, Tranel P J. Characterization of de novo transcriptome for waterhemp (Amaranthus tuberculatus) using GS-FLX 454 pyrosequencing and its application for studies of herbicide target-site genes. Pest Manag Sci. 2010; 66:1042-1052. doi: 10.1002/ps.2006
28. Duhoux A, Carrere S, Duhoux A, Delye C. Transcriptional markers enable identification of rye-grass (Lolium sp.) plants with non-target-site-based resistance to herbicides inhibiting acetolactate-synthase. Plant Sci. Elsevier Ireland Ltd; 2017; 257:22-36. doi: 10.1016/j.plantsci.2017.01.009
29. Gardin J A C, Gouzy J, Carrère S, Délye C. ALOMYbase, a resource to investigate non-target-site-based resistance to herbicides inhibiting acetolactate-synthase (ALS) in the major grass weed Alopecurus myosuroides (black-grass). BMC Genomics. BMC Genomics; 2015; 16:590. doi: 10.1186/s12864-015-1804-x
30. Hu J, Tranel P J, Stewart Jr. CN, Yuan J S. Molecular and genomic mechanisms of non-target-site herbicide resistance. In: Stewart C N, editor. Weedy and Invasive Plant Genomics. Ames, IA; 2009. pp. 149-161.
31. Gaines T A, Lorentz L, Figge A, Herrmann J, Maiwald F, Ott M C, et al. RNA-Seq transcriptome analysis to identify genes involved in metabolism-based diclofop resistance in Lolium rigidum. Plant J. 2014; 78:865-876. doi: 10.1111/tpj.12514
32. Rouse C E, Saski C A, Noorai R E, Shankar V, Burgos N R. Multiple herbicide resistance involving quinclorac in Echinochloa colona involves adaptive co-evolution of abiotic stress- and xenobiotic detoxification genes. University of Arkansas. 2017.
33. Rouse C, Saski C, Noorai R E, Lawton-Rauh A L, Shankar V, Burgos N R. Propanil resistance in multiple-resistant Echinochloa colona may be a concerted total plant response by abiotic and biotic stress genes. 2017.
34. Grossmann K, Scheltrup F. Selective Induction of 1-Aminocyclopropane-1-carboxylic Acid (ACC) Synthase Activity Is Involved in the Selectivity of the Auxin Herbicide Quinclorac between Barnyard Grass and Rice. Pestic Biochem Physiol. 1997; 58:145-153. doi: 10.1006/pest.1997.2290
35. Santiago J, Rodrigues A, Saez A, Rubio S, Antoni R, Dupeux F, et al. Modulation of drought resistance by the abscisic acid receptor PYL5 through inhibition of clade A PP2Cs. Plant J. 2009; 60:575-588. doi: 10.1111/j.1365-313 X.2009.03981.x
36. Jung C, Seo J S, Han S W, Koo Y J, Kim C H, Song S I, et al. Overexpression of AtMYB44 Enhances Stomatal Closure to Confer Abiotic Stress Tolerance in Transgenic Arabidopsis. Plant Physiol. 2007; 146:623-635. doi: 10.1104/U.S. Plant Pat. No. 107.110981
37. Krishnaswamy S, Verma S, Rahman M H, Kav N N V. Functional characterization of four APETALA2-family genes (RAP2.6, RAP2.6L, DREB19 and DREB26) in Arabidopsis. Plant Mol Biol. 2011; 75:107-127. doi: 10.1007/s11103-010-9711-7
38. Liu J, Macarisin D, Wisniewski M, Sui Y, Droby S, Norelli J, et al. Production of hydrogen peroxide and expression of ROS-generating genes in peach flower petals in response to host and non-host fungal pathogens. Plant Pathol. 2013; 62:820-828. doi: 10.1111/j.1365-3059.2012.02683.x
39. Nanda A K, Andrio E, Marino D, Pauly N, Dunand C. Reactive oxygen species during plant-microorganism early interactions. J Integr Plant Biol. 2010; 52:195-204. doi: 10.1111/j.1744-7909.2010.00933.x
40. Asai T, Tena G, Plotnikova J, Willmann M R, Chiu W-L, Gomez-Gomez L, et al. MAP kinase signalling cascade in Arabidopsis innate immunity. Nature. 2002; 415:977-983. doi: 10.1038/415977a
41. Takahashi F, Mizoguchi T, Yoshida R, Ichimura K, Shinozaki K. Calmodulin-Dependent Activation of MAP Kinase for ROS Homeostasis in Arabidopsis. Mol Cell. Elsevier Inc.; 2011; 41:649-660. doi: 10.1016/j.molcel.2011.02.029
42. Xing Y, Cao Q, Zhang Q, Qin L, Jia W, Zhang J. MKK5 regulates high light-induced gene expression of Cu/Zn superoxide dismutase 1 and 2 in arabidopsis. Plant Cell Physiol. 2013; 54:1217-1227. doi: 10.1093/pcp/pct072
43. Jing Y, Lin R. The VQ Motif-Containing Protein Family of Plant-Specific Transcriptional Regulators. Plant Physiol. 2015; 169:371-378. doi: 10.1104/pp. 15.00788
44. Liang S C, Hartwig B, Perera P, Mora-García S, de Leau E, Thornton H, et al. Kicking against the PRCs-A Domesticated Transposase Antagonises Silencing Mediated by Polycomb Group Proteins and Is an Accessory Component of Polycomb Repressive Complex 2. PLOS Genet. 2015; 11:1-26. doi: 10.1371/journal.pgen. 1005660
45. Rouse C E, Burgos N R. Physiological assessment of multiple herbicide-resistant Echinochloa colona reveals the complexity of quinclorac resistance. 2017.
46. Hauser F, Waadt R, Schroeder J I. Evolution of abscisic acid synthesis and signaling mechanisms. Curr Biol. Elsevier Ltd; 2011; 21: R346-R355. doi: 10.1016/j.cub.2011.03.015
47. Tuteja N, Mahajan S. Calcium signaling network in plants: an overview. Plant Signal Behav. 2007; 2:79-85. doi: 10.4161/psb.2.2.4176
48. Niu L, Liao W. Hydrogen Peroxide Signaling in Plant Development and Abiotic Responses: Crosstalk with Nitric Oxide and Calcium. Front Plant Sci. 2016; 7:1-14. doi: 10.3389/fpls.2016.00230
49. Downton W, Loveys B R, Grant W J R. Stomatal closure fully accounts for the inhibition of photosynthesis by abscisic acid. New Phytol. 1988; 108:263-266. doi: 10.1111/j. 1469-8137.1988.tb04161.x
50. Dann E K, Diers B W, Hammerschmidt R. Suppression of sclerotinia stem rot of soybean by lactofen herbicide treatment. Phytopathology. 1999; 89:598-602. doi: 10.1094/PHYTO. 1999.89.7.598
51. Leyser O. Auxin, self-organisation, and the colonial nature of plants. Curr Biol. Elsevier Ltd; 2011; 21: R331-R337. doi: 10.1016/j.cub.2011.02.031
52. Grossmann K. Auxin herbicides: Current status of mechanism and mode of action. Pest Manag Sci. 2010; 66:113-120. doi: 10.1002/ps.1860
53. Vigueira C C, Olsen K M, Caicedo A L. The red queen in the corn: agricultural weeds as models of rapid adaptive evolution. Heredity (Edinb). Nature Publishing Group; 2013; 110:303-311. doi: 10.1038/hdy.2012.104
54. Lunn J E, Delorge I, Figueroa C M, Van Dijck P, Stitt M. Trehalose metabolism in plants. Plant J. 2014; 79:544-567. doi: 10.1111/tpj. 12509
55. Schluepmann H, Pellny T, van Dijken A, Smeekens S, Paul M. Trehalose 6-phosphate is indispensable for carbohydrate utilization and growth in Arabidopsis thaliana. Proc Natl Acad Sci USA. 2003; 100:6849-6854. doi: 10.1073/pnas.1132018100
56. Duan C-G, Wang X, Xie S, Pan L, Miki D, Tang K, et al. A pair of transposon-derived proteins function in a histone acetyltransferase complex for active DNA demethylation. Cell Res. Nature Publishing Group; 2017; 27:226-240. doi: 10.1038/cr.2016.147
57. Ricci W A, Zhang X. Public Service by a Selfish Gene: A Domesticated Transposase Antagonizes Polycomb Function. PLOS Genet. 2016; 12:10-12. doi: 10.1371/journal.pgen.1006014
58. Supek F, Bošnjak M, Škunca N, Šmuc T. Revigo summarizes and visualizes long lists of gene ontology terms. PLOS One. 2011; 6. doi: 10.1371/journal.pone.0021800
59. Robinson M D, McCarthy D J, Smyth G K. edgeR: A Bioconductor package for differential expression analysis of digital gene expression data. Bioinformatics. 2009; 26:139-140. doi: 10.1093/bioinformatics/btp616
60 McCarthy D J, Chen Y, Smyth G K. Differential expression analysis of multifactor RNA-Seq experiments with respect to biological variation. Nucleic Acids Res. 2012; 40:4288-4297. doi: 10.1093/nar/gks042
61. UniProt: the universal protein knowledgebase. Nucleic Acids Res. 2017; 45: D158-D169. doi: 10.1093/nar/gkw1152
62. Kanehisa M, Furumichi M, Tanabe M, Sato Y, Morishima K. KEGG: New perspectives on genomes, pathways, diseases and drugs. Nucleic Acids Res. 2017; 45: D353-D361. doi: 10.1093/nar/gkw1092

### Example 4—Cytochrome P450 Involved in Herbicide Resistance

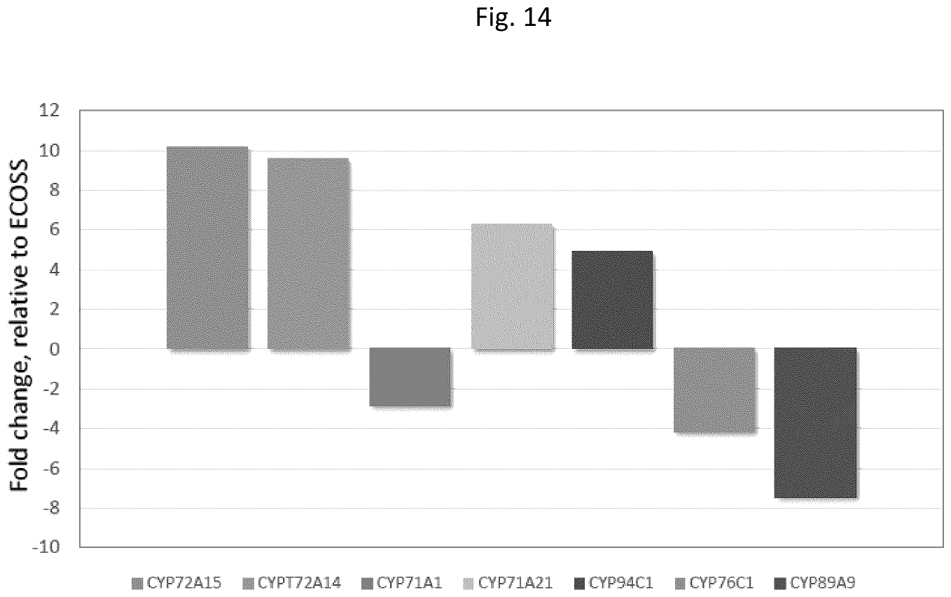

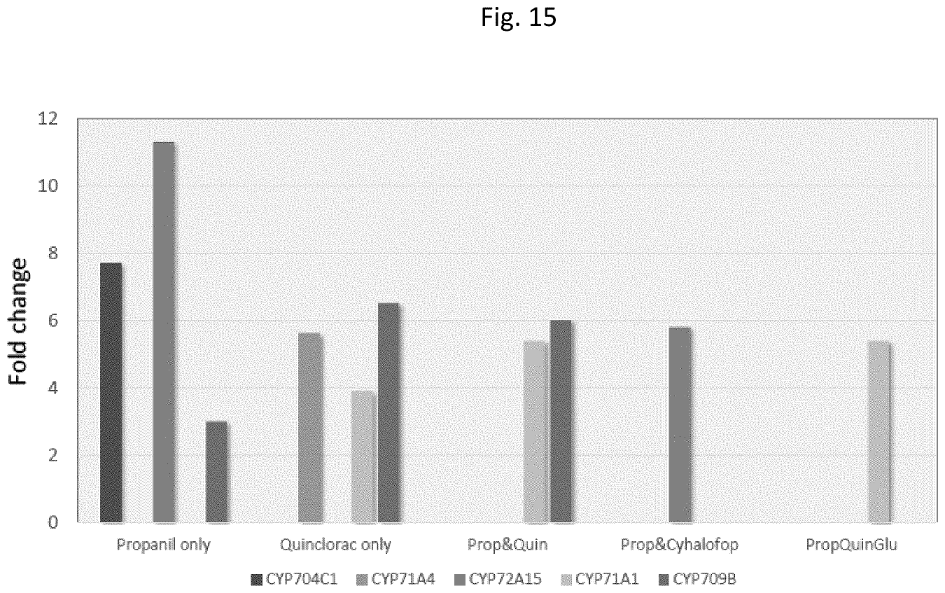

E. colona is often found in rice fields and is often resistant to herbicides. The detoxification reactions mediated by the cytochrome P450s are not always herbicide structure specific. Thus some of the cytochrome P450s endow multiple herbicide resistance. We found upregulation of several cytochrome P450 genes in resistant plants (e.g., ECO45) as compared to susceptible plants (e.g., ECOSS). As shown in FIG. 14, CYP72A15, CYP72A14, CYP71A21, CYP94C1 were all upregulated in ECO45 plants as compared to ECOSS plants. This increase in expression is correlated with increased resistance to various tested herbicides, as shown in FIG. 15. Thus, increased expression of these cytochromes is likely important for herbicide resistance in these plants.

### Example 5—Validation of Candidate Genes Implicated in Herbicide Detoxification and Abiotic Stress Tolerance in Response to Herbicides

The differential induction of the candidate genes identified by the transcriptome study had to be verified in other multiple-resistant weed populations to be confident about the involvement of these genes in the evolution of extreme high resistance to herbicides.

### Results

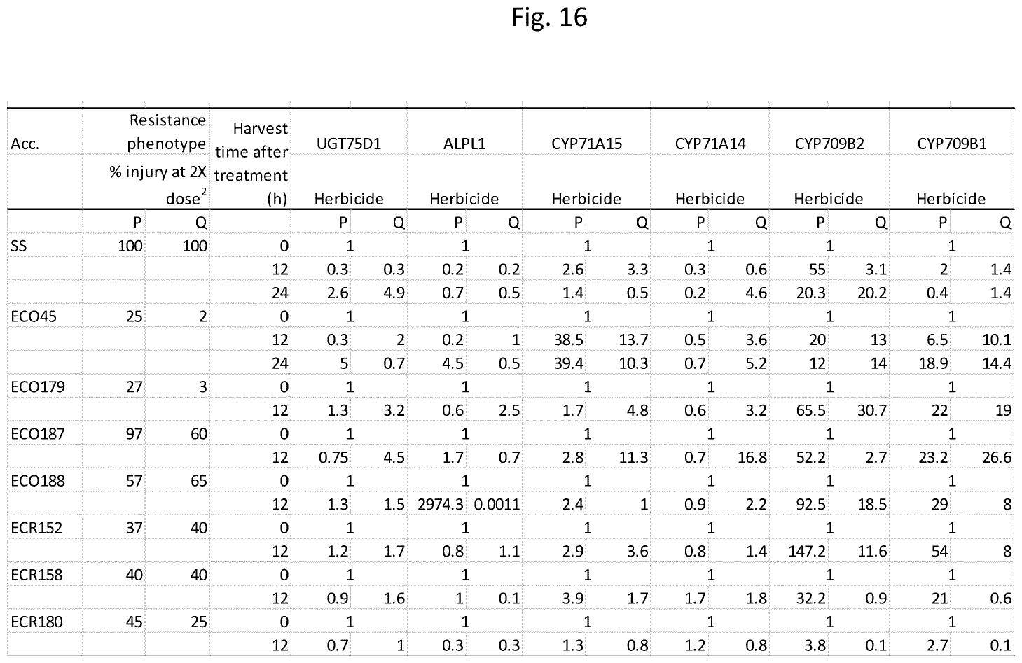

Susceptible plants were killed (100%) with 2 X doses of propanil and quinclorac (FIG. 16). The 2 X dose of propanil (4480 g ai/ha) injured the resistant Echinochloa species tested by only 27-57% with the exception of ECO187, which was injured 97%. The 2 X dose of quinclorac injured the resistant species 2-65%. Therefore, all the resistant accessions were confirmed to have different levels of resistance to quinclorac and all, except ECO187, were also resistant to propanil.

The ALPL1-like gene was induced by propanil in ECO45 and ECO188 and by quinclorac in ECO179 (FIG. 16). Little is known about the function of ALPL1, but it is known to be an epigenetic factor (potentially a transcription factor) that is associated with abiotic stress mitigation. ALPL1 was highly (160-fold) and constitutively expressed in ECO45 as well as in ECO179, ECO187, and ECR152, but not in ECO188 (Table 8). Without herbicide treatment, this putative stress-protection gene is already expressed at a much higher level in 5 of 6 resistant accessions tested, compared to the susceptible standard.

TABLE 8
Constitutive change in R genotypes: gene expression
fold-change relative to the susceptible standard

| | Time of | | | | | | |
|---|---|---|---|---|---|---|---|
| | harvest after | | | | | | |
| Accession | treatment (h) | UGT75D1 | ALPL1-like | TPS9 | TPS6 | TPP1 | TPP9 |
| SS | 0 | 1.0 | 1.0 | 1.0 | 1.0 | 1.0 | 1.0 |
| ECO45 | 0 | 3.4 | 160.0 | 4.1 | 1.6 | 2.2 | 5.3 |
| ECO179 | 0 | 1.5 | 157.0 | 16.4 | 0.3 | 0.2 | 6.0 |
| ECO187 | 0 | 13.0 | 153.5 | 7.7 | 0.1 | 0.7 | 4.8 |
| ECO188 | 0 | 1.5 | 2.1 | 2.6 | 2.1 | 0.4 | 0.6 |
| ECR152 | 0 | 0.9 | 160.5 | 5.4 | 0.2 | 0.1 | 4.3 |

Herbicide detoxification, or metabolic degradation, is mediated by cytochrome P450 genes driving Phase I detoxification reactions and by conjugation genes driving Phase II reactions. CYP71A15 was induced by propanil in the majority the of accessions tested, with the highest induction (39-fold) being in ECO45, which is one of the two most propanil-resistant accessions (FIG. 16). This gene was also induced by quinclorac in the majority of the tested accessions. In contrast, CYP71A14 was induced by quinclorac in 4 of 7 quinclorac-resistant accessions. The level of induction was similar to that in the susceptible accession, except in ECO187. This gene was not induced by propanil in any of the accessions tested. Therefore, CYP71A14 is not involved in metabolic detoxification of propanil, nor is it driving widespread resistance to quinclorac. CYP71 is the largest family of cytochrome P450s [2] and there is a large diversity in substrate reactivity and gene function in this family. In fact, one member of this family, CYP71AM1, was recently identified as being involved in the biosynthesis of the allelochemical sorgoleone in sorghum [3]. Allelochemicals are secondary compounds that are produced by plants for protection against biotic stressors. The GbCYP71 gene of Ginkgo biloba has been shown to function as mitigator of biotic and abiotic stresses, including salt stress [4]. CYP709B1 and CYP709B2 were induced by both propanil and quinclorac in 5 of 7 resistant accessions analyzed. Both genes were induced by propanil in all resistant accessions tested. Therefore, the co-induction of these CYP709B genes may be partly responsible for multiple resistance to propanil and quinclorac. However, high induction of CYP709B2 alone could not endow resistance to propanil based on its high induction in the susceptible plants.

### CONCLUSIONS

Cytochrome P450 genes in the CYP71A and CYP709B families are involved in multiple resistance of E. colona and E. crus-galli to propanil and quinclorac. However, the extent of their involvement varies across the tested weed populations. Each of these genes contributes to plant health through herbicide detoxification, stress mitigation, or both.

### Materials and Methods

### Gene Expression Analysis by Reverse Transcriptase (RT)-qPCR

To verify the roles of several primary candidate genes, we conducted gene expression analysis experiments using four E. colona and three E. crus-galli accessions with multiple resistance to propanil and quinclorac and a susceptible E. colona standard(S). Seeds of ECO85 (S), ECO45, ECO152, ECO158, ECO179, ECO180, ECO187 and ECO188 lines were pregerminated in trays filled with commercial potting soil. Approximately one week after planting, seedlings were transplanted into square pots measuring 7.6 cm wide and 10.2 cm tall, containing a 1:3 mixture by volume of commercial potting soil and field soil (Captina silt loam-fine-silty, siliceous, active, mesic typic fragiudult). Each pot contained one plant, and each treatment was replicated three times. All plants were treated at the 2-3 leaf growth stage. The herbicides were applied using a laboratory spray chamber fitted with a motorized boom, calibrated to deliver 187 L/ha spray volume. Tissues of the treated plants were harvested at 0 (before spraying), 12, and 24 hours after treatment and immediately frozen in liquid nitrogen and stored at −80° C. until processed. Total RNA was isolated using an E.Z.N.A® RNA isolation kit (Omega Biotek, Norcross, GA) and converted to cDNA using RevertAid First Strand cDNA Synthesis Kit (Thermo Fisher Scientific, Waltham, MA, USA) according to manufacturer's instructions using 1 μg of total RNA for each sample. Multiple plants were pooled in all genotypes and in all biological replicates. The differences in transcript abundance of the following genes were measured by RT-qPCR using the iCycler Real-Time PCR Detection System (Bio-Rad): ALPL1-Like, UGT75D1, UGT73D1, UGTI, TPP1, TPP9, TPS6, TPS9, CYP709B1, CYP709B2, CYP72A14, and CYP72A15. Each qPCR reaction contained 1 X iQ SYBR Green Superpmix (2×) (BIO-RAD), 1 μl of cDNA (diluted 1:5), and 0.5 μM of gene-specific primers (Table 9). The relative expression levels of each gene were calculated using the 2^{-ΔΔct} algorithm [1] by normalizing to the expression of the O. sativa elongation factor 2 gene, which was used as an internal control. Fold change was calculated either relative to 0 hr of each line (represents inductive expression) or to 0 hr of S (represents constitutive expression level in R lines). Each sample was analyzed in two or three technical replicates using at least two biological replicates.

### Determination of Resistance Level

The same Echinochloa lines were established as described above, except that five seedlings were transplanted per pot. At the 3-leaf stage, the seedlings were treated with various doses ranging from 0.0625 X to 32 X of propanil (1 X dose=2240 g ai/ha) and quinclorac (1 X dose=560 g ai/ha). Propanil was applied with 0.25% v/v non-ionic surfactant and quinclorac was applied with 1% v/v crop oil concentrate. The herbicides were applied using a laboratory spray chamber fitted with a motorized boom and calibrated to deliver 187 L/ha spray volume. Injury from herbicide treatments was evaluated visually 3 wk after treatment. The experiment was repeated once.

TABLE 9
RNA primers used for
the qRT-PCR gene expression assay

| Gene | Primer sequence |
|---|---|
| ALPL1-like | F-AACGCCTTCCAGATCTTCG |
| | (SEQ ID NO: 40) |
| | R-GAGGAGGTTGTGTGCGATCT |
| | (SEQ ID NO: 41) |
| TPP1 | F-CACCTGACGAAGATCCTGCT |
| | (SEQ ID NO: 42) |
| | R-ATCTTCCTGCCTTGTGCATT |
| | (SEQ ID NO: 43) |
| TPP9 | F-GTGACAAGGTGTTGGGCTTC |
| | (SEQ ID NO: 44) |
| | R-CTTGTTGTCGTGGTTGTTGG |
| | (SEQ ID NO: 45) |
| TPS6 | F-AACGATCGAGGACAAGGAGA |
| | (SEQ ID NO: 46) |
| | R-GACGCTCTCCAGATGGTCA |
| | (SEQ ID NO: 47) |
| TPS9 | F-GGCTGCTGAGATCTTTGCTT |
| | (SEQ ID NO: 48) |
| | R-CGTCAGGCTCTTGAGCATCT |
| | (SEQ ID NO: 49) |
| UGT75D1 | F-GCTCACTTTCCCGTTCCAG |
| | (SEQ ID NO: 50) |
| | R-GTGGTGGAGAATGTGACGAG |
| | (SEQ ID NO: 51) |
| UGT1 | F-ACATGTCCCAGGTGAAGCTC |
| | (SEQ ID NO: 52) |
| | R-GGAGAGCAGCAGCGTGTAG |
| | (SEQ ID NO: 53) |
| UGT73D1 | F-GTGAACACGTTCCTGGACCT |
| | (SEQ ID NO: 54) |
| | R-TCGACGTCTTTGTTCACGAG |
| | (SEQ ID NO: 55) |
| CYP709B1 | F-GTCGTCAAGCAGGTGCTCTT |
| | (SEQ ID NO: 56) |
| | R-CAGTGAGGACGAGACCCTTG |
| | (SEQ ID NO: 57) |
| CYP709B2 | F-GCCTGAGAGGTTCGAGTACG |
| | (SEQ ID NO: 58) |
| | R-CGATCATCGCAAAGTTCTGA |
| | (SEQ ID NO: 59) |
| CYP72A14 | F-TCGGTGGCATCAAATATCCT |
| | (SEQ ID NO: 60) |
| | R-GAACTTGCCTGCGTCTTTTC |
| | (SEQ ID NO: 61) |
| CYP72A15 | F-CCAGTGAGCTGATACGCAGA |
| | (SEQ ID NO: 62) |
| | R-GACGTCGCCTGTGAGATTTT |
| | (SEQ ID NO: 63) |

### REFERENCES FOR EXAMPLE 4

1. Livak K J and Schmittgen T D. Analysis of relative gene expression data using real-time 928 quantitative PCR and the 2-AACT method. Methods. 2001; 25:402-408. 929doi: 10.1006/meth.2001.1262
2. Nelson D and Werck-Reichhart D. A P450-centric view of plant evolution. The Plant Journal (2011) 66, 194-211 doi: 10.1111/j.1365-313 X.2011.04529
3. Pan Z, Baerson S R, Wang M, Bajsa-Hirschel J, Rimando A M, Wang X, Dhammika N, Nanayakkara P, Noonan B P, Fromm M E, Dayan F E, Khan I A, SO Duke. A cytochrome P450 CYP71 enzyme expressed in Sorghum bicolor root hair cells participates in the biosynthesis of the benzoquinone allelochemical sorgoleone. New Phytologist (2018) 218:616-629 doi: 10.1111/nph.15037
4. LIU X, CAO F, CAIJ, WANG H. The Molecular Cloning and Expression Analysis of a CYP71 Gene in Ginkgo biloba L. Not Bot Horti Agrobo, 2016, 44 (1): 77-84. DOI: 10.15835/nbha441

### Example 6—Validation of Candidate Genes Implicated in Abiotic Stress Tolerance in Response to Drought Stress

In a previous experiment, we observed that the multiple-resistant population ECO45 can regrow when rewatered shortly after desiccation while other accessions could not recover. This raised the question of whether some genes endowing multiple resistance to herbicides can also endow tolerance to drought (among other abiotic stresses), or whether abiotic stress tolerance genes play a role in the evolution of multiple resistance to herbicides via nontarget-site resistance (NTSR) mechanisms. Answering this question will allow us to better understand resistance evolution and to adjust weed management practices in view of environmental stresses that may accelerate resistance evolution.

This follow-up study was conducted to obtain phenotypic, physiological, and molecular data from multiple-resistant E. colona accessions (ECO45 and ECO179) in order to verify and understand the ability of these accessions to tolerate drought stress.

### Results

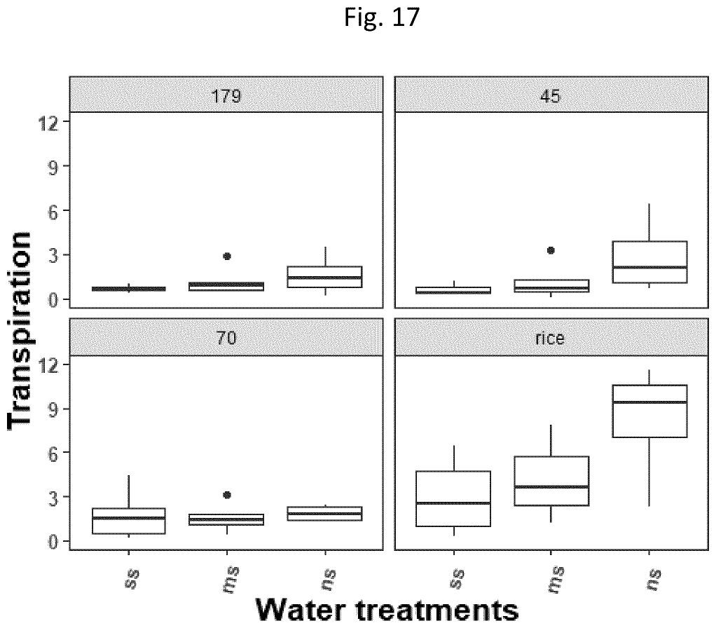

This study is ongoing. The stress treatments had been implemented and the plants are almost mature. A preliminary gene expression analysis under severe drought stress revealed a 6-to 8-times higher induction of UGTI in the multiple-resistant accessions ECO45 and ECO179, and a 2- to 6-fold induction of UGT73D1 relative to the susceptible E. colona (Table 10). Further, CYP709B genes were induced at least 4 times more in the resistant accessions than in the susceptible one. Notably, these gene expression differences resulted in some observable physiological adaptations to drought stress in the resistant accessions. Our preliminary data showed that E. colona transpired at least 4 times less than rice regardless of resistance trait at field capacity and at moderate drought treatment (FIG. 17). This difference narrowed under severe drought stress, wherein the multiple-resistant ECO45 and ECO179 minimized transpirtation more than rice did and numerically more than susceptible ECO70 did.

### CONCLUSION

UGTI and UGT73D1 help mitigate drought stress. However, the mechanism by which this occurs in resistant E. colona needs further study. CYP709B1 and CYP709B2 also help mitigate drought stress in addition to mitigating the harmful effects of propanil and quinclorac. The specific mechanism of action by which these genes protect plants against drought needs further investigation. At the whole-plant level, the multiple-resistant E. colona plants tended to minimize their transpiration rate under severe water stress more than the susceptible plants (ECO70). Expressing these traits in crops would greatly improve drought stress tolerance.

### Materials and Methods

### Experimental Design

The experimental design was two-factor factorial, with four replications and treatments arranged in a randomized complete block design. Factor A was the test plants: 1) susceptible standard ECO70-SS; 2) extreme high resistance ECO179; 3) resistant standard ECO45-RR; and 4) rice cultivar ‘Diamond’. Factor B was the drought stress treatment: 1) no water stress-saturated (i.e., the normal habitat of rice and Echinochloa); 2) moderate stress-withhold water until 50% of field capacity (FC), then rewater back to FC; 3) severe stress-withhold water until 50% of FC, ±10% and maintain at this level for two weeks.

### Plant Establishment

Seeds were pregerminated in flats filled with commercial potting soil. One week after planting, uniform-size seedlings were transplanted into 6-inch pots filled with the same weight of a mixture of 4 parts field soil and 1 part commercial potting medium, at 5 seedlings per pot. Rice was transplanted at 2 seedlings per pot. The plants were kept well-watered in the greenhouse. At the three leaf stage, ECO45 and ECO179 were sprayed with a 4 X dose of quinclorac to confirm that all plants were equally resistant. (Note: The 1 X dose of quinclorac is 560 g ai/ha.) Two weeks after quinclorac application, all treatments were thinned to one healthy plant per pot. The plants were fertilized once every two weeks following transplanting.

### Implementation of Drought Stress Treatments

Drought treatments were initiated when the plants started to transition to the reproductive stage (i.e., when the primary tiller started to boot or extend its flag leaf). The field capacity weight was determined by saturating the pots in the evening, allowing gravitational water to drain overnight and weighing the pots in the early morning while transpiration is minimal. Thereafter, water was withheld until each treatment reached its designated water stress level, while the control plants were maintained at field capacity moisture. The stress treatments were implemented over a two-week period after which the plants were maintained at field capacity until maturity.

TABLE 10
Response of candidate genes to severe drought stress in Echinochcloa colona plants: susceptible standard
(SS-Eco70), EcoR45 and EcoR179, Altheimer Laboratory greenhouse, Fayetteville, AR.

| | Drought | | | | | | ALPL1- |
|---|---|---|---|---|---|---|---|
| Accession | Treatment | TPS6 | TPS9 | TPP1 | TPP9 | UGT1 | LIKE |
| SS | no drought | 1 | 1 | 1 | 1 | 1 | 1 |
| SS | Severe drought | 2.978 | 1.396 | 3.404 | 8.118 | 2.368 | 12.723 |
| ECO45 | no drought | 1 | 1 | 1 | 1 | 1 | 1 |
| ECO45 | Severe drought | 2.435 | 0.263 | 0.429 | 2.694 | 16.267 | 3.874 |
| ECO179 | no drought | 1 | 1 | 1 | 1 | 1 | 1 |
| ECO179 | Severe drought | 1.508 | 1.196 | 0.285 | 2.746 | 12.062 | 1.334 |
| | Drought | UGT | | | | | |
| Accession | Treatment | 73D1 | GST6 | CYP709B1 | CYP709B2 | CYP72A14 | CYP72A15 |
| SS | no drought | 1 | 1 | 1 | 1 | 1 | 1 |
| SS | Severe drought | 1.455 | 2.731 | 2.491 | 2.462 | 0.941 | 2.486 |
| ECO45 | no drought | 1 | 1 | 1 | 1 | 1 | 1 |
| ECO45 | Severe drought | 2.112 | 1.046 | 11.75 | 8.906 | 1.423 | 1.627 |
| ECO179 | no drought | 1 | 1 | 1 | 1 | 1 | 1 |
| ECO179 | Severe drought | 5.956 | 1.58 | 1.786 | 0.695 | 1.141 | 3.473 |

1 Pooled RNA from four replications.
Highlighted in bold font are upregulation responses compared to their respective non-stressed plants.

We claim:

1. A plant cell modified to increase as compared to a control plant cell the expression or enzyme activity of at least one protein selected from the group consisting of trehalose phosphate synthetase (TPS), trehalose phosphate phosphatase (TPP), Protein ALP1-like (At3g55350 or ALPL1), Glycosyltransferase 75D1 (UGT75D1), Cytochrome P450 709B2 (CYP709B2), Cytochrome P450 709B1 (CYP709B1) and Cytochrome P450 72A15 (CYP72A15), wherein the TPS protein comprises any one of SEQ ID NOS: 1-16 (JungleRice TPS) or a variant or homolog of any one of SEQ ID NOS: 1-16 comprising at least 80% sequence identity to any one of SEQ ID NOS: 1-16, the TPP protein comprises any one of SEQ ID NOS: 17-21 (JungleRice TPP) or a variant or homolog of any one of SEQ ID NOS: 17-21 comprising at least 80% sequence identity to SEQ ID NOS: 17-21, the ALPL1 protein comprises any one of SEQ ID NOS: 22-27 (JungleRice ALPL1) or a variant or homolog of any one of SEQ ID NOS: 22-27 comprising at least 80% sequence identity to any one of SEQ ID NOS: 22-27, the UGT75D1 protein comprises any one of SEQ ID NOS: 28-30 (JungleRice UGT75D1), or a variant or homolog of any one of SEQ ID NOS: 28-30 comprising at least 80% sequence identity to any one of SEQ ID NOS: 28-30, the CYP709B2 comprises any one of SEQ ID NOS: 31-33 (JungleRice CYP709B2), or a variant or homolog of any one of SEQ ID NOS: 31-33 comprising at least 80% sequence identity to any one of SEQ ID NOS: 31-33, the CYP709B1 comprises SEQ ID NO: 34 (JungleRice CYP709B1), or a variant or homolog of SEQ ID NO: 34 comprising at least 80% sequence identity to SEQ ID NO: 34 and the CYP72A15 protein comprises any one of SEQ ID NOS: 35-39 (JungleRice CYP72A15), or a variant or homolog of any one of SEQ ID NOS: 35-39 comprising at least 80% sequence identity to any one of SEQ ID NOS: 35-39.

2. The plant cell of claim 1, wherein the plant cell is modified to increase the expression of at least one protein selected from the group consisting of the ALPL1 protein, the UGT75D1 protein, the CYP709B2 protein, the CYP709B1 protein and the CYP72A15 protein.

3. The plant cell of claim 2, wherein the plant cell is modified to increase the expression of the TPS protein and the TPP protein.

4. The plant cell of claim 1, wherein the plant cell is modified to increase the expression of the TPS protein, the TPP protein, the ALPL1 protein, the UGT75D1 protein, the CYP709B2 protein, the CYP709B1 protein, and the CYP72A15 protein.

5. The plant cell of claim 1, wherein the enzyme activity or expression of each protein is increased by at least 30% as compared to a control plant cell.

6. The plant cell of claim 1, wherein the plant cell comprises a heterologous promoter operably connected to a polynucleotide encoding the at least one protein.

7. The plant cell of claim 1, wherein the plant cell comprises a hypermorphic mutation in a polynucleotide encoding the at least one protein.

8. The plant cell of claim 1, wherein the plant cell is selected from the group consisting of a corn plant cell, a cotton plant cell, a soybean plant cell, a rice plant cell, a sorghum (sweet or grain) cell, a canola cell, a wheat cell, a sugarbeet cell, a tomato plant cell, a cucurbit plant cell, and a sunflower cell.

9. A plant comprising the plant cell of claim 1.

10. The plant of claim 9, wherein the plant has improved resistance to an herbicide as compared to a control plant.

11. The plant of claim 10, wherein the herbicide comprises a photosynthesis inhibitor or a synthetic auxin.

12. The plant of claim 10, wherein the herbicide comprises at least one of quinclorac, propanil, protoporphyrinogen IX oxidase (PPO) inhibitors, acetolactate synthase (ALS) inhibitors, photosystem I inhibitors, acetyl coenzyme A carboxylase (ACCase) inhibitors, fluorpyrauxifen, or carotenoid biosynthesis inhibitors, HPPD or DOXp synthase inhibitors.

13. The plant of claim 9, wherein the plant has improved abiotic stress tolerance as compared to a control plant.

14. The plant of claim 9, wherein the plant is selected from the group consisting of corn plant, a cotton plant, a soybean plant, a rice plant, a sorghum (sweet or grain) plant, a canola plant, a wheat plant, a sugarbeet plant, a tomato plant, a cucurbit plant, and a sunflower plant.

15. A seed, part, progeny or asexual propagate of a plant comprising the plant cell of claim 1.

16. A method comprising planting the seeds of claim 15 in an area.

17. The method of claim 16, wherein the area is at risk of drought and having below average precipitation.

18. The method of claim 16, further comprising applying an herbicide to the area.

19. The method of claim 18, wherein the herbicide comprises a photosynthesis inhibitor or a synthetic auxin.

20. The method of claim 18, wherein the herbicide comprises quinclorac or propanil.


SEQUENCE LISTING (63 sequences)

SEQ ID NO: 1 - protein, 248 aa, Echinochloa colona
EEVLRTLLNCDLIGFHTFDYARHFLSCCSRMFGLEYQSKRGYMGLEYFGRTVGIKIMPMGIHMGQLQSVLRLPETEQKVAELRQRFEGKSVLLGVDDTDIFKGIDLKLLAFETMLRMHPKWQGRAVLVQIANPPRGKGKELEAIQVEIRQSCERINREFGQTGYSPVVFIDRKVSSAERLAYYTIAECVVVTAVRDGMNLTPYEYIVCRQGIPGSDSVPEVSGPKKSMLVVSEFIGCSPSLSGAIRVN

SEQ ID NO: 2 - protein, 292 aa, Echinochloa colona
MIDRILGLGPCSRKKSMLVVSEFIGCSPSLSGAIRVNPWNIDSVADAMDSALEMPEGEKVLRHEKHHKYVSTHDVGYWANSFLQDLERTCLDHNRRRCWGIGFGLKFRVVALDPNFKKLAVEHLVSAYRRTTKRIILLDYDGTLMPQTSFGKGPTSKTIDMLNSLCRDKNNMVFLVSTKSRATLDEWFTPCENLGLAAEHGYFLRLRRDAEWERCVPVIDCSSWKQIAEPVMKTYTETTDGSTIEDKETAIVWSYEDADPDFGSCQAKELHDHLESVLANEPVSVKAGDRKS

SEQ ID NO: 3 - protein, 629 aa, Echinochloa colona
FFLHSPFPSSEIIRTIPVREELLRALLNADLVGFHTYDYARHFLSACCRLLGVNNRSRRGYIGLDYFGRTVVVKILPAGVDMEQLREVLSSPETAAKAEEIATRFAGRKVLLGVDDVDLFKGIGLKLVAMEKLLEARKELRGELVLVQINNPARSSGRDIDTVREEVQVMRDRINARFSSPGYEPIVTIDDPLTMHEKLAFYTSADICIVTAVRDGLSRIPYIYTVCRQEGPIAGDVADGAPRESAIVLSEFVGCSPSLSGAVPINPWNAEGVADGMSTALMLNELERQMRQEKHYSYVSKHDIVYWGQSLDQDLQRASKDNASMNMLNMGLAMNFRIIVLAPNFKKLLPGDINPSYNQTGNRLILLDYDGTVTPEEVMIRNPSQELIGVLNNLCSDPNNTVFVVSGRSKDELAGWLAPCERLGISAEHGYFTRWSRDSPWESANLAVKFDWKNIAEPIMKHYTDATDGSYIETKETALVWHYEEADPDFGSCQAKELQDHLLSVLSKEPVAVKSGHKIVEVNPKDVGKGTAVRSLIAAMGARGNLPDFILCVGDDGSDEDMFAAITTPSSNYAFPEAAEIFACTLGKKPSLAKYYLEDTDDVLKMLKSLTASSPLGGALQGQVSFEDT

SEQ ID NO: 4 - protein, 188 aa, Echinochloa colona
GMVSRSYSNLLELASGGGGGGGGGEPLPSLGRRRIPRVVTASGIVPDLDASDDDADAASAASDHSSSHAPRERVIIVANQLPVRAARRPGGAGAGWDFAWDEDSLLLQVRDSLRAHHGRGDVEFVYVGGLRDDVPPADHDAVAHELLEGFRCVPTFLPADLRSRFYHGFCKQQLWPLFHYMLPLSPEL

SEQ ID NO: 5 - protein, 372 aa, Echinochloa colona
LDLANGNLSALDYGGGGGGGGGGGGGRPPRARRMQRTMTTPGTLVELDEETAGSVASDVQSSLAADRLIVVANTLPVRGERRPDGRGWAFSWDEDSLLFHLRDGLPDDMEVLYVGSLRADVPPAEQDDVAQALLERFRCVPAFLPKDLCDRFYHGFCKQTLWPLFHYMIPYNPARGGPFDRSQWEAYVLANKLFSQRVIEVLNPEDDYVWIHDYHLLALPSFLRRRFNRLRIGFFLHSPFPTSELYRSLPVRDEILKSLLNCDLIGFHTFDYARHFLSCCSRMLGLEYQSKRGYIGLDYFGRTVGIKIMPVGINMVQLQSLLQQPDLERQVAELRNQFHRKTVLLGVDDMDIFKGIDLKILAFEQMLKTHPK

SEQ ID NO: 6 - protein, 222 aa, Echinochloa colona
CSSDLFDYARHFLSACSRLLGLDYQSKRGYIGIEYYGRTVTVKILPVGIDMGQLRSVVTAPETGDVVRRVAEMYKGRRLMIGVDDVDLFKGIGLKFLAMEQLLVEHPELRGRAVLVQIANPARSEGRDVQGVQDEARAISARVNARFGTPGYTPIVLLDGAVTAHEKAAYYAAAECCVVSAVRDGLNRIPYIYTVCRQESNALGDDDAPKRSVIVLSEFVGC

SEQ ID NO: 7 - protein, 148 aa, Echinochloa colona
MKCYVSPCRWTRDEEWQKSTHTADFGWMQMAEPVMNLYTEATDGSYIETKETALVWHHQDADPGFGSSQAKEMLDHLESVLSKEPVSVKSGQFIVEVKPQGVSKGLVAENILTLMKEKGQQADFVLCIGDDRSDEDMFENIADVMKRN

SEQ ID NO: 8 - protein, 423 aa, Echinochloa colona
RSIPGSESVPEVSGPKKSMLVVSEFIGCSPSLSGAIRVNPWNIETTAEALNEAISMSEQEKQLRHGKHYRYVSTHDVTYWSKSFVQDLERACKDHFRKPCWGIGLGFGFRVVALDPHFTKLNLDTIVMSYERAKSRAILLDYDGTLVPQASINKEPSAEIVRIINTLCSDSNNIVFIVSGRSRDSLGKLFASCPKLGIAAEHGYFLRWTRDEEWQKSTHTADFGWMQMAEPVMNLYTESTDGSYIETKETALVWHHQDADPGFGSSQAKEMLDHLESVLANEPVAVKSGQFIVEVKPQGVSKGLVAENILTLMKEKGQQADFVLCIGDDRSDEDMFENIADVMKRNIVAPKTPLFACTVGQKPSKARFYLDDTFEVVNMLSSLADASDPDSLTELEDDLATSISSIDIGHEPPKLGNRRDEGS

SEQ ID NO: 9 - protein, 489 aa, Echinochloa colona
QGIPGSDSVPEVSGPKKSMLVVSEFIGCSPSLSGAIRVNPWNIETTAEALNEAISMSEQEKQLRHGKHYRYVSTHDVTYWSKSFVQDLERACKDHFRKPCWGIGLGFGFRVVALDPHFTKLNLDTIVMSYERAKSRAILLDYDGTLVPQASINKEPSAEIVRIINTLCSDSNNIVFIVSGRSRDSLGKLFASCPKLGIAAEHGYFLRWTRDEEWQKSTHTADFGWMQMAEPVMNLYTESTDGSYIETKETALVWHHQDADPGFGSSQAKEMLDHLESVLSKEPVSVKSGQFIVEVKPQVCDIYLFQSFHKGPCFFTDVSQILAPSMDNKDTFFFFLMTLPSLVFSFLNYVSMYISSIILFVLQGVSKGLVAENILTLMKEKGQQADFVLCIGDDRSDEDMFENIADVMKRNIVAPKTPLFACTVGQKPSKAKFYLDDTFEVVNMLSSLADASDPDSSIIELEDDLATSISSIDIGDEPPKLGNRRDEGS

SEQ ID NO: 10 - protein, 210 aa, Echinochloa colona
RCVPTFLPADLRSRFYHGFCKQQLWPLFHYMLPLSPELGGRFDRLLWQAYVSVNKIFADKILEVISPDEDFVWVHDYHLMVLPTFLRKRFNRVKLGFFLHSPFPSSEIYKTLPVREELLRSLLNADLIGFHTFDYARHFLSCCSRMLGLKYESQRGYIALEYYGRTVTIKILPVGIHLEQLQSVLNLKDTGLKVAELMKQFCHRNRLLLL

SEQ ID NO: 11 - protein, 380 aa, Echinochloa colona
LEGFRCVPTFLPADLRSRFYHGFCKQQLWPLFHYMLPLSPELGGRFDRLLWQAYVSVNKIFADKILEVISPDEDFVWVHDYHLMVLPTFLRKRFNRVKLGFFLHSPFPSSEIYKTLPVREELLRSLLNADLIGFHTFDYARHFLSCCSRMLGLKYESQRGYIALEYYGRTVTIKILPVGIHLEQLQSVLNLQETGDKVAELMKQFCHRNRLLLLGVDDMDIFKGISLKLLAFEQLLVQHPEWRGRVVLVQIANPARGKGKDVKEVQEESDAMVKRINDAFRQPDYEPVMLIDKPLQFYERMAYYVVAECCLVTAVRDGMNLIPYEYIIARQGNEMIDRILGLGPSSRKKSMLVVSEFIGCSPSLSGAIRVNPWNIDSVAD

SEQ ID NO: 12 - protein, 380 aa, Echinochloa colona
LEGFRCVPTFLPADLRSRFYHGFCKQQLWPLFHYMLPLSPELGGRFDRLLWQAYVSVNKIFADKILEVISPDEDFVWVHDYHLMVLPTFLRKRFNRVKLGFFLHSPFPSSEIYKTLPVREELLRSLLNADLIGFHTFDYARHFLSCCSRMLGLKYESQRGYIALEYYGRTVTIKILPVGVHLEQLQSVLNLPETGVKVAELMKLFCHRNRLLLLGVDDMDIFKGISLKLLAFEQLLVQHPEWRGRVVLVQIANPARGKGKDVKEVQEESDAMVKRINEAFGQTDYQPVILIDKPLQFYERMAYYVVAECCLVTAVRDGMNLIPYEYIIARQGNEMIDRILGLGPSSRKKSMLVVSEFIGCSPSLSGAIRVNPWNIDSVAD

SEQ ID NO: 13 - protein, 244 aa, Echinochloa colona
ETAIVWSYEDADPDFGSCQAKELHDHLESVLANEPVSVKAGLNHVEVKPQVSFGLLLHTTATATVHLLSSRMCHFTSCSKSQVLDKILLVIASVDWFAGREQGRGGEADPVDDAGARGPAGLRAVRGRRPVRRGHVRGDQHGDGSTIEDKETAIVWSYEDADPDFGSCQAKELHDHLESVLANESVSVKAGLNHVEVKPQGVSKGVVAKRILSTMRERGDLPDFVLCVGDDRSDEDMFEVISTA

SEQ ID NO: 14 - protein, 286 aa, Echinochloa colona
MPSLSCHNLLDLAAADDAPPLLSSPTPLRLPRVMSVAASPASSPTSPPPRRVIVSHRLPLRAAPDPAAPFGFAFSVDAGTVAYQLRSGLPAGAPVLHIGTLPAAAAEAASDELSNYLVANFSCLPVYLPGDLHHRFYHGFCKHYLWPLLHYLLPLTPSSLGGLPFQRALYHSFLSANRAFADRLTEVLAPDEDLVWIHDYHLLALPTFLRKRFPRAKVGFFLHSPFPSSEIFRTIPVRDDLVRALLNADLVGFHTFDYARHFLSACSRLLGLDYQSKRGYIGIEYY

SEQ ID NO: 15 - protein, 271 aa, Echinochloa colona
MPSLSCHNLLDLAAADETPLPSPTPLRLPRVMSVAASPASPTSPSPPAPPRRVIVSHRLPLRAAPDPAAPFGFAFTVDAGTVAYQLRSGLPASAPVLHIGTLPAAAAEAASDELSNYLVTNFSCLPVYLPSDLHHRFYHGFCKHYLWPLLHYLLPLTPSSLGGLPFQRSLYHSFLSANRAFADRLTEVLAPDEDLVWIHDYHLLALPTFLRKRLPRAKVGFFLHSPFPSSEIFRTIPVRDDLVRALLNADLVGFHTFDYARHFLSACSRLL

SEQ ID NO: 16 - protein, 230 aa, Echinochloa colona
NQLPVVARRRADGRGWVFSWDEDSLLLRLRDGVPDEMEVFFVGSLRADVPPAEQDEVSQTLIDGFRCAPVFLPPELNERFYHHFCKRYLWPLFHYMLPSAPPLSPTTEAAIPGDGGRFDRSAWEAYVLANKYFYEKVVEVINPEDDYVWVHDYHLMALPTFLRRRFNRLRIGFFLHSPFPSSEIYRTLPVREEVLRTLLNCDLIGFHTFDYARHFLSCCSRMFGLEYQSK

SEQ ID NO: 17 - protein, 173 aa, Echinochloa colona
MRAAVRNVAKYFPTAIVSGRSRKKVFEFVKLKELYYAGSHGMDIVTSVAEHNTDKCKESNLFQPAWEFLPMIDEVSKSLLEVTSGIEGASVENNKFCVSVHYRNVAEKDWGVVARLVNKVLEAYPRLKVANGRMVLEVRPVIDWDKGKAVEFLLQSLGLDDSESVIPIYIGDD

SEQ ID NO: 18 - protein, 271 aa, Echinochloa colona
SALTSFKQIVANAQGRKIAVFLDYDGTLSPIVDDPDKAFMSPVMRAAVRNVAKYFPTAIVSGRSRKKVFEFVKLKELYYAGSHGMDIVTSVAEHNTDKCKEANLFQPAWEFLPMIDEVSKSLLEVTSGIEGASVENNKFCVSVHYRNVAEKDWGVVARLVKEALEAYPRLKVTNGRMVLEVRPVIDWDKGKAVEFLLQSLGLDDSESVIPIYIGDDRTDEDAFKVLRERKCGYGILVSQVPKHTEAFYSLRDPSEVLGFLNSLVRWKKHSL

SEQ ID NO: 19 - protein, 143 aa, Echinochloa colona
MDMSTSSPVITDPLSISPPLLGSLTSNLMPFSVMSGGCSSPGMNMSASRRKIEEVLVNGLLDAMKSSSPRKKHNLAFGQGNTPDEDPAYSAWMSKCPSALTSFKQIVANAQGRKIAVFLDYDGTLSPIVDDPDKAFMSPVMRA

SEQ ID NO: 20 - protein, 258 aa, Echinochloa colona
MTKHGVVMGPEDAVVAAAAARHFSFPPPRTTGGVESCRKLAAAQVDLSAAAVMGSWLDSMKASSPRHRIMAPLVPGAGAGAGDAEHDDWMERHPSALERFDAVAAAAKGKQVAVFLDYDGTLSPIVEDPDCAVMTDEMREAVRGVAARFPTAIVSGRCCDKVLGFVRLEELYYAGSHGMDIRGPTADPNNHDNKEESSAGGKKSVLCQPAAEFLPVIQEVHGALVAAVEASIPGAKVENNKFCLSVHFRCVEEGAWAA

SEQ ID NO: 21 - protein, 121 aa, Echinochloa colona
GALVAAVEASIPGAKVENNKFCLSVHFRCVEEGAWAALFDRVRSVLKDYPGLRLTQGRKVLEVRPIIKWDKGRALEFLLQSLGYAERSDVFPIYIGDDRTDEDAFKVLRGRGQGAGILVTK

SEQ ID NO: 22 - protein, 217 aa, Echinochloa colona
MDERDRIRFEYLNNKIWKNDVTCVNMLRLNKASFFHFCKLFRDRGLLQDTIHVCVEQQVAIFFNTVGHNLKNRLVGTNYDRSGEAVSRYFNKVLRAVGELRNEFIRPPSSATPSKITGNPRWDPYFKDCIGAIDGTHVRASVPKDLEPAFHGRKSHCTQNVMAAVDFDLRFTYVLAGWAGTAHDALVLRNALERENGLRVPQGNCRYIPYLNIPCHL

SEQ ID NO: 23 - protein, 185 aa, Echinochloa colona
ITVQGVVDAAGAFTDVCIGWPGSMSDADVLDRSALYAQRGAAGRLQGQWVVGGAGYPLMDWLLVPYTHQNMTWAQHVFNERVDGVRAVARDAFRRLKARWGCLQKRTEVKLQDLPVVLGACCVLHNICERAGDAVDPDNAFQIFDDDMIAENPVRSPAAATARDEIAHNLLHRNRAGGPAGFLLK

SEQ ID NO: 24 - protein, 170 aa, Echinochloa colona
FRMSRATFEAVCEELGAAVAKEDTMLRAAIPVRQRVAVCIWRLATGEPLRLVSKRFGLGISTCHKLVLEVCAAIKAVLMPKAVQWPDPAADAAAAFEAASGIAGVVGAMYTTHIPIIAPKANVAAYYNRRHTERNQKTSYSITVQGVVDAAGAFTDVCIGWPGSMPDDQV

SEQ ID NO: 25 - protein, 199 aa, Echinochloa colona
FRMSRATFEAVCEELGAAVAKEDTMLRAAIPVRQRVAVCIWRLATGEPLRLVSKRFGLGISTCHKLVLEVCAAIKAVLMPKAVQWPDPAAAAASSFEAASGIAGVVGAMYTTHIPIIAPKANVAAYYNRRHTERNQKTSYSITVQGVVDAAGAFTDVCIGWPGSMSDADVLDRSALYAQRGAAGRLQGQWVVGGAGYPL

SEQ ID NO: 26 - protein, 212 aa, Echinochloa colona
MEASFRGRKNHATQNVMAAVDFDLRFTYVLAGWEGTAHDALVLRDALERENGLRVPQGKFYLVDARYGAKPGFLPPFRGVRYHLNEWGNNPVQNEKELFNLRHSSLRVTVERAFGSLKRRFKILDDATPYFPFPTQVDIVVACCIIHNWVIQDGGDELIIQENNWTPTLTPIVLLKYYRQWKLREVDQRLWLVCVNGHQHNPLLFSVSFLTL

SEQ ID NO: 27 - protein, 370 aa, Echinochloa colona
CALPIYNDVQFRQRYRMRRHLFLQIVQTLSIWSPYFCQRSDAFGKVGFSPLHKCTVAMRMLAYGTPAHMWDENFRMAESTIIECMKTFCQGVIANFGEKYLRRPTSEDIRRLLHIGEARGFPGMLGSLDCMHWQWRNCPKAWRGQFTRGDIKHPTVMLEAVASHDLWIWHAFFGVAGSNNDINVLNRSPLFTEVLQGRAPEVHFTVNGNEYKMGYYLADGIYPEWAAFAKTIPLPQCEKDKLYAEHQEGARKDVERAFGVLQARFAILRNPSRMWQLQSLSEIMYACIILHNMIVEDERDTFRVRYDDNYESEYDQGSSSTPLAGFGHGPIHEFSRLLQIEEDIRDRDMHRQLKEDLVEHIWQRFGRNQA

SEQ ID NO: 28 - protein, 380 aa, Echinochloa colona
MTPRPHFLVLTFPFQGHIAPALRLARRLLAAAPAALVTFSTTEAAHRRMFPANKPDAAKGAADGDGDDSRLEFLPFSDGTEAGYVRSSDPGSFNAYMASFHASGARSVAGIVDALAARGRPVSRVVYTILLPWAAGVARERGVPSALYWIQPAVVFAVYHRFFHGHAGAVAELHRRGDPSLAVELPGLPPLSVRDLPTFLTESTDPANYFHAVFLTFRDLFDALDTETPRATVLVNSCEELEVGTLTAVARHDVLPIGPVLPAGGYDETSIFKRDHAKYMEWLDTKPGDDGDGAARRAAPRAGGEREAVPPCGPEGQQGGARRSQGGDGRAPQERHGGGVVRPGVGAVARGDGVLRDALRVELGGGERGERRAHGGRAQG

SEQ ID NO: 29 - protein, 107 aa, Echinochloa colona
KDGRYLEWLDSKPAKSVVYISFGSSSVMSKSQVAEIAEAMARIKRPFLWVVRKDNCKDNKEDDAAIRKLTAAAGSSTETEGMVVEWCDQARVLSRPSVACFVTHGGW

SEQ ID NO: 30 - protein, 380 aa, Echinochloa colona; identical to SEQ ID NO: 28

SEQ ID NO: 31 - protein, 195 aa, Echinochloa colona
FFAGHDTTSHLLTWASFLLSTHPEWQDKLREEVRRECGDEIPTGDMLNKLKLVNMFLLETLRLYGPVSAIQRKASSDLELGGVRVPEDTILTIPIATIHRDKEVWGEDAGEFKPERFENGVTRAAKHPNALLSFSSGPRSCIGQNFAMIEAKAVVAMILQRFTLELSPKYVHAPMDVLTLRPRHGLPMLLKRLEV

SEQ ID NO: 32 - protein, 346 aa, Echinochloa colona
VAAAVAAAVASWAFNALVVHLVWRPYAVTRRLRAQGVRGPGYRFFSGNLGDIKRLRAEGAGVTLDAGDHDFIPMVQPHFRKWISLYGRTFVYWTGARPNVCVADVNVVKQVLFDRNGLYPKNLMNPHISRLLGKGLVLTDGDDWKRHRKVVHPAFSMDKLKMMTVTMSDCAQSMMSEWEAQLAKVTGAVEVELSSRFEELTADVISHTAFGSSYNEGKRVFLAQRELQFLAFSTVFDVQIPAFRYLPTEKNLKTWKLDKQVRGMLMDIIKTRLATKDTAAGYGNDLLGLMLEACAAPEHGEAPALSMDEIIDECKTFFFAGHDTTSHLLTWASFLLSTHPEWQDKL

SEQ ID NO: 33 - protein, 154 aa, Echinochloa colona
PTGDMLNKLKLVNMFLLETLRLYGPVSAIQRKASSDLELGGVRVPEDTILTIPIATIHRDKEIWGEDAGEFKPERFEYGVTRAAKHPNALLSFSSGPRSCIGQNFAMIEAKAVVAMILQRFTLELSPKYVHAPMDVLTLRPRHGLPMLLKRLEV

SEQ ID NO: 34 - protein, 183 aa, Echinochloa colona
VAAAVAAAVASWAFNALVVHLVWRPYAVTRRLRAQGVRGPGYRFFSGNLGDIKRLRAEGAGVTLDAGDHDFIPMVQPHFRKWISLYGRTFVYWTGARPNVCVADVNVVKQVLFDRTGLYPKNLMNPHISRLLGKGLVLTDGDDWKRHRKVVHPAFSMDKLKVHIYIYRIEYAICRRVVRALLG

SEQ ID NO: 35 - protein, 350 aa, Echinochloa colona
MDDVVAAAAAAAASPSPWILLQGLLALLVVWGAYRAAERCWLRPRRLDRALRAQGLSGTEYCFPAGDLKENGRLNEEARSRPMKLCHDVVPRVMPHLLNTVKEHGNICITWFGPIPRVIITEAELVRDILSNKFGHFEKFTNKRLGKLLALGLASYDGEKWAKHRRILNPAFHLEKLKRMLPAFSTCCTELTDRWESKLAGSDGSYEVDIWPEFQNLTGDVISRTAFGSSFMEGRRIFQLQAEQAERVIKAFQYMYIPGFLFLPTKNNRRMKEINGEIEGILRGMIEKRERAIEKGEASGNDLLGLLLQSNMDSGKGSLRMSTEDVIEECKLFYFAGMETTSVLLTWTMI

SEQ ID NO: 36 - protein, 226 aa, Echinochloa colona
RSSRIGKLLANGVVNHDGEKWAKHRRILNPAFHHEKIKRMLPVFSACCTETIIRWENSMPSEGSSEIDVWPEFQNLTGDVISRTAFGSSYQEGMKIFQLQGEQAERLVQSFQTLFIPGYWFLPTKNNRRMREIDREICKILREIIGKREKAMKNGETNNDDLLGLLLESNMRQSNGNAKLGLTTEDVIEECKLFYFAGMETTSVLLTWTLIVLSMHPEWQERAREE

SEQ ID NO: 37 - protein, 126 aa, Echinochloa colona
RSSRIGKLLANGVVNHDGEKWAKHRRILNPAFHHEKIKRMLPVFSSCCTETIIRWENSMPSEGSSEIDVWPEFQNLTGDVISRTAFGSSYQEGMKIFQLQAEQAERLVQSFQTLFIPGYWLVLLYF

SEQ ID NO: 38 - protein, 529 aa, Echinochloa colona
MVLGALAGEFASTPWSFLIYVLLGALLLWKAARLLEPLWWAPRRLERALRAQGLSGTSYRFLNGDLKEYGRANKEAWSRPLPLRCHDITAYVAPFICAAVREHGKTCFTWFGPIPKVTITDPDLARDVMSNKFGHFEKPKFPAMSKLFADGVANYEGEKWVKHRRILNPAFHLEKLKLMLPAFSACCEELVSRWAQSLGPDGCCELDVEPELQTLTGDVISRTAFGSSYLEGRKIFQLQAEQAERLMSIVHKFGIPGYMSLPTKNNRRMRQIKGEVETILRGLIGKRMQSMKEGEPTKDDLLGLLLESNMKETAVNGQSSSLGMTIEEVMEECKLFYFAGMETTSVLLTWTMILLSMHPEWQDRAREEVLGLFGKNKPGYDGLSRLKIVTMILYEVLRLYPPAIAFSRKTYKEMEIGDATYPAGVILELPVLQIHHDPDIWGSDVHEFRPERFAEGIAKASRDRLAFFPFGWGPRICIGQNFALLEAKMALSMMLQSFEFELAPSYTHAPRTVIMLRPMHGAQIKLRAI

SEQ ID NO: 39 - protein, 257 aa, Echinochloa colona
TPYRFLTGDLKEYGRANKEAWSRPLPLRCHDIAAYVAPFICAAVREHGETCFTWFGPIPKVTITDPDLARDVMSNKFGHFEKPKFPALSKLFADGVANYEGEKWVKHRRILNPAFHLEKLKLMLPAFSACCEELVSRWAQSLGPDGCCELDVEPELQTLTGDVISRTAFGSSYLEGRKIFQLQAEQAGRLMSIVHKFGIPGYMSLPTKNNRRMRQIKSEVETILRGLIGKRMQAMKEGEPTKDDLLGLLLESNMKET

SEQ ID NO: 40 - DNA, 19 bp, synthetic construct
  misc_feature 1..19: note = Synthethic- Forward primer for ALPL1-like
  source 1..19: mol_type = other DNA
aacgccttccagatcttcg

SEQ ID NO: 41 - DNA, 20 bp, synthetic construct
  misc_feature 1..20: note = Synthethic- Reverse primer for ALPL1-like
  source 1..20: mol_type = other DNA
gaggaggttgtgtgcgatct

SEQ ID NO: 42 - DNA, 20 bp, synthetic construct
  misc_feature 1..20: note = Synthethic- Forward primer for TPP1
  source 1..20: mol_type = other DNA
cacctgacgaagatcctgct

SEQ ID NO: 43 - DNA, 20 bp, synthetic construct
  misc_feature 1..20: note = Synthethic- Reverse primer for TPP1
  source 1..20: mol_type = other DNA
atcttcctgccttgtgcatt

SEQ ID NO: 44 - DNA, 20 bp, synthetic construct
  misc_feature 1..20: note = Synthethic- Forward primer for TPP9
  source 1..20: mol_type = other DNA
gtgacaaggtgttgggcttc

SEQ ID NO: 45 - DNA, 20 bp, synthetic construct
  misc_feature 1..20: note = Synthethic- Reverse primer for TPP9
  source 1..20: mol_type = other DNA
cttgttgtcgtggttgttgg

SEQ ID NO: 46 - DNA, 20 bp, synthetic construct
  misc_feature 1..20: note = Synthethic- Forward primer for TPS6
  source 1..20: mol_type = other DNA
aacgatcgaggacaaggaga

SEQ ID NO: 47 - DNA, 19 bp, synthetic construct
  misc_feature 1..19: note = Synthethic- Reverse primer for TPS6
  source 1..19: mol_type = other DNA
gacgctctccagatggtca

SEQ ID NO: 48 - DNA, 20 bp, synthetic construct
  misc_feature 1..20: note = Synthethic- Forward primer for TPS9
  source 1..20: mol_type = other DNA
ggctgctgagatctttgctt

SEQ ID NO: 49 - DNA, 20 bp, synthetic construct
  misc_feature 1..20: note = Synthethic- Reverse primer for TPS9
  source 1..20: mol_type = other DNA
cgtcaggctcttgagcatct

SEQ ID NO: 50 - DNA, 19 bp, synthetic construct
  misc_feature 1..19: note = Synthethic- Forward primer for UGT75D1
  source 1..19: mol_type = other DNA
gctcactttcccgttccag

SEQ ID NO: 51 - DNA, 20 bp, synthetic construct
  misc_feature 1..20: note = Synthethic- Reverse primer for UGT75D1
  source 1..20: mol_type = other DNA
gtggtggagaatgtgacgag

SEQ ID NO: 52 - DNA, 20 bp, synthetic construct
  misc_feature 1..20: note = Synthethic- Forward primer for UGT1
  source 1..20: mol_type = other DNA
acatgtcccaggtgaagctc

SEQ ID NO: 53 - DNA, 19 bp, synthetic construct
  misc_feature 1..19: note = Synthethic- Reverse primer for UGT1
  source 1..19: mol_type = other DNA
ggagagcagcagcgtgtag

SEQ ID NO: 54 - DNA, 20 bp, synthetic construct
  misc_feature 1..20: note = Synthethic- Forward primer for UGT73D1
  source 1..20: mol_type = other DNA
gtgaacacgttcctggacct

SEQ ID NO: 55 - DNA, 20 bp, synthetic construct
  misc_feature 1..20: note = Synthethic- Reverse primer for UGT73D1
  source 1..20: mol_type = other DNA
tcgacgtctttgttcacgag

SEQ ID NO: 56 - DNA, 20 bp, synthetic construct
  misc_feature 1..20: note = Synthethic- Forward primer for CYP709B1
  source 1..20: mol_type = other DNA
gtcgtcaagcaggtgctctt

SEQ ID NO: 57 - DNA, 20 bp, synthetic construct
  misc_feature 1..20: note = Synthethic- Reverse primer for CYP709B1
  source 1..20: mol_type = other DNA
cagtgaggacgagacccttg

SEQ ID NO: 58 - DNA, 20 bp, synthetic construct
  misc_feature 1..20: note = Synthethic- Forward primer for CYP709B2
  source 1..20: mol_type = other DNA
gcctgagaggttcgagtacg

SEQ ID NO: 59 - DNA, 20 bp, synthetic construct
  misc_feature 1..20: note = Synthethic- Reverse primer for CYP709B2
  source 1..20: mol_type = other DNA
cgatcatcgcaaagttctga

SEQ ID NO: 60 - DNA, 20 bp, synthetic construct
  misc_feature 1..20: note = Synthethic- Forward primer for CYP72A14
  source 1..20: mol_type = other DNA
tcggtggcatcaaatatcct

SEQ ID NO: 61 - DNA, 20 bp, synthetic construct
  misc_feature 1..20: note = Synthethic- Reverse primer for CYP72A14
  source 1..20: mol_type = other DNA
gaacttgcctgcgtcttttc

SEQ ID NO: 62 - DNA, 20 bp, synthetic construct
  misc_feature 1..20: note = Synthethic- Forward primer for CYP72A15
  source 1..20: mol_type = other DNA
ccagtgagctgatacgcaga

SEQ ID NO: 63 - DNA, 20 bp, synthetic construct
  misc_feature 1..20: note = Synthethic- Reverse primer for CYP72A15
  source 1..20: mol_type = other DNA
gacgtcgcctgtgagatttt